(19) United States
(12) Patent Application Publication
(10) Pub. No.: US 2024/0180891 A1
(43) Pub. Date: Jun. 6, 2024

(54) SYNERGISTIC MODULATORS OF ALPHA-DICARBONYL DETOXIFICATION AND THEIR USE FOR INDUCING WEIGHT LOSS AND THE TREATMENT OF DIABETIC PATHOLOGIES

(71) Applicant: Buck Institute for Research on Aging, Novato, CA (US)

(72) Inventors: Pankaj Kapahi, Kensington, CA (US); Neelanjan Bose, Burlingame, CA (US); Lauren Wimer, Novato, CA (US); Jyotiska Chaudhuri, Petaluma, CA (US)

(73) Assignee: Buck Institute for Research on Aging, Novato, CA (US)

(21) Appl. No.: 18/268,190

(22) PCT Filed: Dec. 20, 2021
(86) PCT No.: PCT/US2021/064400

§ 371 Date: Jun. 16, 2023

Related U.S. Application Data
(60) Provisional application No. 63/129,445, filed on Dec. 22, 2020.

(51) Int. Cl. A61K 31/465; A61K 31/4172; A61K 31/4415; A61K 31/445; A61K 31/51; A61P 3/04; A61P 3/10; A61P 25/28

(52) U.S. Cl. CPC A61K 31/465; A61K 31/4172; A61K 31/4415; A61K 31/445; A61K 31/51; A61P 3/04; A61P 3/10; A61P 25/28

(57) ABSTRACT

In various embodiments compositions and methods are provided for inducing or increasing weight loss or reducing or preventing weight gain in a mammal. In certain embodiments the methods comprise administering to the mammal a combination of at least two agents selected from the group consisting of lipoic acid and/or a lipoic acid analog, nicotinamide and/or a nicotinamide analog, thiamine and/or a thiamine analog, piperine and/or a piperine analog, pyridoxamine and/or a pyridoxamine analog. In certain embodiments the combination of agents provides a synergistic effect.

### CROSS-REFERENCE TO RELATED APPLICATIONS

This application claims benefit of and priority to U.S. Ser. No. 63/129,445, filed on Dec. 22, 2020, which is incorporated herein by reference in its entirety for all purposes.

### STATEMENT OF GOVERNMENTAL SUPPORT

[Not Applicable]

### BACKGROUND

Obesity is a significant healthcare burden, increasing the risk of several diseases, including diabetes, cardiovascular diseases, certain cancers, severe COVID-19, and reducing life expectancy. Obesity affects both developed and emerging countries; one-third of the US population is obese (BMI>30). Excess caloric intake and increasingly sedentary lifestyles play a role in fueling obesity. Whereas wearable activity trackers and lifestyle programs enable people to increase physical activity, dietary improvements remain challenging for most individuals. For one thing, the oversupply of calorically dense foods facilitates excess consumption, resulting in weight gain. For many, increased adiposity drives progressive unresponsiveness to homeostatic cues that normally maintain weight stability, such as the adipokine leptin. Leptin resistance, in turn, fuels obesity. Another driver of obesity is the high prevalence of sugary foods and beverages in our dietary landscape. This, coupled with the existence of ‘hedonic mechanisms’ in the brain that enhance pleasure and drive addiction to sugar, has been proposed to partially explain the ˜100 fold increase in sugar intake amongst Americans over the last 200 years (see, e.g., Hoebel et al. (2009) J. Addict Med. 3: 33-41; Wiss et al. (2018) Front. Psychiatry 9: 545; Vucetic & Reyes (2010) Wiley Interdiscip. Rev. Syst. Biol. Med. 2: 577-593). Both “homeostatic” and “hedonic” mechanisms drive food intake and were evolutionarily critical for building fat reserves for times when food was scarce and unreliable. However, the fact that we continue to respond to these programs despite an ample food supply in today's world leads to obesity.

Leptin resistance is linked to specific functional deficiencies in the leptinergic melanocortin system within the mediobasal hypothalamus that emerge in response to chronic dietary excess and disrupt homeostatic regulation of food intake. By contrast, the molecular underpinnings of the hedonic mechanisms driving carbohydrate addiction are complex. Although dopamine and acetylcholine signaling have received great interest (see, e.g., Hoebel et al. (2009) J. Addict Med. 3: 33-41; Wiss et al. (2018) Front. Psychiatry 9: 545), we remain limited by an incomplete determination of the molecular pathways mediating sugar addiction and/or carbohydrate preference and lack the ability to leverage hedonic pathways to control food intake for therapeutic purposes. With a mindset of overcoming this gap to gain new insights into mitigating obesity, we have recently identified methylglyoxal (MGO) and its glycated metabolic end-products (AGEs) as important potential targets.

Hyperglycemia results in several metabolic and biochemical perturbations, including the elevation of a series of highly reactive α-dicarbonyl compounds (α-DCs), such as MGO (see, e.g., Henning et al. (2014) J. Biol. Chem. 289: 28676-28688; Singh et al. (2014) The Korean journal of physiology & pharmacology: official journal of the Korean Physiological Society and the Korean Society of Pharmacology. 18:1-14). MGO is an unavoidable byproduct of anaerobic glycolysis (see, e.g., Rabbani & Thornalley (1979) Nat. Protoc.:9: 1969-1979; Lange et al. (2012) Adv. Urol. 819202, doi:10.1155/2012/819202; Thornalley et al. (2003) Biochem. J. 375: 581-592), generated spontaneously when glucose breaks down into dihydroxyacetone phosphate (DHAP) and glyceraldehyde 3 phosphate (G3P). MGO reacts indiscriminately with proteins, lipids, and DNA to yield a heterogeneous group of molecules collectively called AGEs (see, e.g., Thornalley et al. (2003) Biochem. J. 375: 581-59; Rabbani et al. (2014) Biochem. Soc. Trans. 42: 511-517; Peppa & Vlassara (2005) Hormones 4: 28-37; Sousa et al. (2013) Biochem. J. 453: 1-15). Accumulated AGEs are sensed by receptors for AGEs (RAGEs), leading to oxidative stress and pro-inflammatory responses (see, e.g., Singh et al. (2014) The Korean journal of physiology & pharmacology: official journal of the Korean Physiological Society and the Korean Society of Pharmacology. 18:1-14; Yan et al. (2008) Nature clinical practice. Endocrinology & metabolism 4: 285-293), but we expect that other receptors also exist (see, e.g., Singh et al. (2001) Diabetologia, 44: 129-146). AGE crosslinking has also been observed in protein aggregates directly related to aging, neurodegeneration in Parkinson's disease (see, e.g., Guerrero et al. (2013) Mol. Neurobiol. 47: 525-536; Li et al. (2012) J. Neurol. Sci. 317:1-5; Salahuddin et al. (2014) Cell Mol. Biol. Lett. 19: 407-437), AD (see, e.g., Kang et al. (2017) Yonsei Med. J. 58: 479-488; de Matos et al. (2018) Med. Res. Rev. 38: 261-324; Jahan & Choudhary (2014) 25: 1267-1284; Mukherjee et al. (2015) Trends Mol. Med. 21: 439-449; Rabbani, & Thornalley (2012) Amino Acids 42:1133-1142), and ATTR amyloidosis (see, e.g., Forbes & Cooper (2013) Physiol. Rev. 93: 137-188; Calcutt et al. (2009) Nat. Rev. Drug Discov. 8: 417-430; Smuda et al. (2014) Biochem. 54: 2500-2507; Monnier & Sell (2006) Rejuvenation Res. 9: 264-273; Sell et al. (1991) Diabetes/Metabolism Rev. 7: 239-251). AGEs and α-DC stress have also been implicated in diabetic complications (see, e.g., Singh et al. (2014) The Korean journal of physiology & pharmacology: official journal of the Korean Physiological Society and the Korean Society of Pharmacology. 18: 1-14; Ma (2016) J. Diabetes Investig. 7: 139-154), such as peripheral neuropathy, nephropathy, cardiomyopathy, and retinopathy (see, e.g., Forbes & Cooper (2013) Physiol. Rev. 93: 137-188; Calcutt et al. (2009) Nat. Rev. Drug Discov. 8: 417-430). Dietary AGEs have also been linked with an increased incidence of diabetes and obesity (see, e.g., Cai et al. (2012) Proc. Natl. Acad. Sci. USA, 109, 15888-15893; Vlassara & Uribarri (2014) Curr. Diabetes Rep. 14: 453). Beyond this, it is reasonable to consider the possibility that MGO, discovered over 100 years ago, and downstream AGEs are also generated even under physiological conditions when glycolytic rates ramp up in specific tissues or cell types (see, e.g., Sousa et al. (2013) Biochem. J. 453: 1-15). Based on our data (see Examples below), believe that such AGEs may, under certain circumstances, act as acute adaptive drivers of food intake when the need arises and also act as chronic, maladaptive drivers of excess food intake in the context of the development of obesity.

### SUMMARY

Despite its high prevalence, obesity continues to be largely managed by encouraging individuals to expend more calories and consume fewer calories. Current medications are also meant to reduce appetite in general and thus reduce food intake. However, these strategies meet with mixed and temporary success, and obesity remains a huge nationwide problem. Notably, food intake is not only driven by “homeostatic” cues that drive hunger in response to food deprivation but is also driven in part by “hedonic” signals that act in the brain to promote the preferential consumption of, and even addiction to, specific nutrients such as carbohydrates including sugar.

Dietary carbohydrates, once metabolized, yield specific byproducts. We determined that one of these, methylglyoxal (MGO), is a precursor for a set of molecules called advanced glycation end-products (AGEs). AGEs are implicated in fueling both type 2 diabetes and obesity, and our work ranging from studies in nematodes to mice indicates that a specific MGO-derived AGE, MG-H1, enhances food intake, resulting in obesity and several of its key consequences.

Moreover, we have seen that lowering AGE levels using a customized chemical cocktail to block their production (anti-AGEs) improves body weight and glucose control in obese and control mice. Interestingly, our data also suggest that the beneficial effects of AGE-lowering strategies may are due to reduction in food intake, specifically carbohydrate rich diet.

Using a combination of Caenorhabditis elegans (C. elegans), mammalian cell lines, and animal models we have developed a platform to discover compounds that lower advanced glycation end products (AGEs). The platform was used to identify a number of compounds that lower AGEs. Several of these compounds are ‘Generally recognized as safe’ (GRAS) and function synergistically to induce or to promote weight loss in mammals. These compounds include lipoic acid, nicotinamide, thiamine, piperine, and pyridoxamine (see, e.g., FIG. 1). It was determined that various combinations of these compounds act synergistically in promoting weight loss. Additionally, it is believed that various combinations of these compounds are effective to reduce food intake, and or to reduce carbohydrate ingestion, and/or to lower sugar, and/or to lower A1C, and/or to slow aging, and/or to extend lifespan or healthspan. Moreover, in view of the data presented herein, it is believed that analogs of these compounds can be similarly efficacious.

Various embodiments provided herein may include, but need not be limited to, one or more of the following:

Embodiment 1: A formulation for inducing or increasing weight loss or reducing or preventing weight gain in a mammal, said formulation comprising:
a combination of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 2: The formulation of embodiment 1, wherein said combination of at least two agents is a synergistic combination.

Embodiment 3: The formulation according to any one of embodiments 1-2, wherein said combination of agents comprises lipoic acid or an analog thereof or a pharmaceutically acceptable salt of said lipoic acid or analog.

Embodiment 4: The formulation of embodiment 3, wherein said combination of agents comprises lipoic acid.

Embodiment 5: The formulation of embodiment 3, wherein said combination of agents comprises a lipoic acid analog.

Embodiment 6: The formulation of embodiment 5, wherein said lipoic acid analog comprises an analog selected from the group consisting of bisnor-lipoic (1,2-dithiolane-3-propanoic), and tetranorlipoic (1,2-dithiolane-3-carboxylic) acid, and a lipoic acid amide (e.g., 6,8-dithiooctanoic amide, 2-(N,N-dimethylamine) ethylamido lipoate, etc.).

Embodiment 7: The formulation according to any one of embodiments 1-6, wherein said lipoic acid or lipoic acid analog comprises a substantially pure R enantiomer.

Embodiment 8: The formulation according to any one of embodiments 1-6, wherein said lipoic acid or lipoic acid analog comprises a substantially pure S enantiomer.

Embodiment 9: The formulation according to any one of embodiments 1-8, wherein said combination of agents comprises nicotinamide or a nicotinamide analog or a pharmaceutically acceptable salt of said nicotinamide or nicotinamide analog.

Embodiment 10: The formulation of embodiment 9, wherein said nicotinamide or nicotinamide analog or nicotinamide metabolite comprises nicotinamide.

Embodiment 11: The formulation of embodiment 9, wherein said nicotinamide or nicotinamide analog or nicotinamide metabolite comprises a nicotinamide metabolite.

Embodiment 12: The formulation of embodiment 11, wherein said nicotinamide metabolite comprise nicotinamide mononucleotide (NMN) or nicotinamide ribonucleoside (NMR).

Embodiment 13: The formulation of embodiment 9, wherein said nicotinamide or nicotinamide analog comprises nicotinic acid (pyridine-3-carboxylic acid), nicotinamide (nicotinic acid amide), or inositol hexanicotinate).

Embodiment 14: The formulation of embodiment 9, wherein said nicotinamide or nicotinamide analog comprises a nicotinamide analog.

Embodiment 15: The formulation of embodiment 14, wherein said nicotinamide or nicotinamide analog comprises a nicotinamide analog nicotinamide analog shown in Table 1.

Embodiment 16: The formulation according to any one of embodiments 9-15, wherein said nicotinamide or nicotinamide analog comprises a substantially pure R enantiomer.

Embodiment 17: The formulation according to any one of embodiments 9-15, wherein said nicotinamide or nicotinamide analog comprises a substantially pure S enantiomer.

Embodiment 18: The formulation according to any one of embodiments 1-17, wherein said combination of agents comprises thiamine or a thiamine analog or a pharmaceutically acceptable salt of said thiamine or thiamine analog.

Embodiment 19: The formulation of embodiment 18, wherein said thiamine or a thiamine analog comprises thiamine.

Embodiment 20: The formulation of embodiment 18, wherein said thiamine or a thiamine analog comprises a thiamine analog.

Embodiment 21: The formulation of embodiment 18, wherein said thiamine analog comprises a thiamine analog selected from the group consisting of pyrithiamine, thiamine disulphide and acetylthiamine, oxythiamine, thiamine diphosphate, oxythiamine diphsophate, neopyrithiamine, 4-methyl-5-(hydroxymethyl)-thiazole, β-(4-Methylthiazolyl-5)-alanine, and 3-(2, 4-dioxo-1, 2, 3, 4-tetrahydro-5-pyrimidinyl) methyl-4-methyl-5-(2-hydroxyethyl) thiazolium nitrate.

Embodiment 22: The formulation according to any one of embodiments 18-21, wherein said thiamine or thiamine analog comprises a substantially pure S enantiomer.

Embodiment 23: The formulation according to any one of embodiments 18-21, wherein said thiamine or thiamine analog comprises a substantially pure R enantiomer.

Embodiment 24: The formulation according to any one of embodiments 1-23, wherein said combination of agents comprises piperine and/or a piperine analog or a pharmaceutically acceptable salt of said piperine and/or piperine analog.

Embodiment 25: The formulation of embodiment 24, wherein said piperine or a piperine analog comprises piperine.

Embodiment 26: The formulation of embodiment 24, wherein said piperine or a piperine analog comprises a piperine analog.

Embodiment 27: The formulation of embodiment 26, wherein said piperine analog comprises a piperine analog selected from the group consisting of 1-(3,4-methylenedioxyphenyl)-penta-2E,4E-dienoic acid methyl ester, 1-E,E-piperinoyl-isobutylamine, and 1-(3,4-methylenedioxyphenyl)-pentanoic acid cyclohexyl amide.

Embodiment 28: The formulation of embodiment 26, wherein said piperine analog comprises a piperine analog shown in Table 2.

Embodiment 29: The formulation of embodiment 26, wherein said piperine analog comprises a piperine analog selected from the group consisting of P1057, P622, P545, P725, P557, P2, P4, P1045, P1090, P8, P594, P1087, P1088, P28, P12, P1118, P1, P1122, P677, P1120, P1121, P6, P1112, P1119, P1117, P1116, P1070, P1114, P569, P32, P5, P1084, P1069, P593, P10, P9, P665, P7, P17, P1123, P11, P1080, P33, P743, P16, P1078, P27, P604, P25, P29, 1073, P26, P30, P707, P752, P22, P670, P23, P21, P546, P3, P581, P15, P636, P20, P737, P649, and P689.

Embodiment 30: The formulation according to any one of embodiments 24-29, wherein said piperine or a piperine analog comprises a substantially pure S enantiomer.

Embodiment 31: The formulation according to any one of embodiments 24-29, wherein said piperine or a piperine analog comprises a substantially pure R enantiomer.

Embodiment 32: The formulation according to any one of embodiments 1-31, wherein said combination of agents comprises pyridoxamine and/or a pyridoxamine analog or a pharmaceutically acceptable salt of said pyridoxamine or pyridoxamine analog.

Embodiment 33: The formulation of embodiment 32, wherein said pyridoxamine or a pyridoxamine analog comprises pyridoxamine.

Embodiment 34: The formulation of embodiment 32, wherein said pyridoxamine or a pyridoxamine analog comprises a pyridoxamine analog.

Embodiment 35: The formulation of embodiment 34, wherein said pyridoxamine analog comprises a pyridoxamine analog selected from the group consisting of salicylamine (o-hydroxybenzylamine), thiosalicylamine (o-mercaptobenzylamine), and 3-hydroxy-4-aminomethylpyridine, 3-hydroxy-4-aminomethylpyridine, 1-methylpyridoxamine chloride, 1-methyl-3-hydroxy-4-aminomethylpyridinium chloride, pyridoxamine phosphate, pyridoxal 5 phosphate, pyridoxine, and an alkyl-pyridoxamine (alkyl-PM).

Embodiment 36: The formulation of embodiment 34, wherein said pyridoxamine analog comprises an alkyl pyridoxamine.

Embodiment 37: The formulation of embodiment 36, wherein said pyridoxamine analog comprises a pentyl-PM, a hexyl-PM, or a heptyl-PM.

Embodiment 38: The formulation according to any one of embodiments 32-37, wherein said pyridoxamine or pyridoxamine analog comprises a substantially pure S enantiomer.

Embodiment 39: The formulation according to any one of embodiments 32-37, wherein said pyridoxamine or pyridoxamine analog comprises a substantially pure R enantiomer.

Embodiment 40: The formulation according to any one of embodiments 1-39, wherein said agents comprising said combination of two or more agents comprises lipoic acid, thiamine, nicotinamide, piperine, and pyridoxamine in substantially equal amounts.

Embodiment 41: The formulation according to any one of embodiments 1-39, wherein said agents comprising said combination of two or more agents comprises lipoic acid, thiamine, nicotinamide, and pyridoxamine in substantially equal amounts, and piperidine in a greater amount the other four compounds.

Embodiment 42: The formulation according to any one of embodiments 1-41, wherein the lipoic acid and/or lipoic acid analog, when present in said formulation, is provided at an amount of at least 50 mg/dose, or at least 100 mg/dose, or at least 150 mg/dose.

Embodiment 43: The formulation of embodiment 42, wherein the lipoic acid and/or lipoic acid analog, when present is administered in an amount of about 150 mg/dose.

Embodiment 44: The formulation according to any one of embodiments 1-43, wherein the nicotinamide and/or nicotinamide analog, or nicotinamide metabolite, when present in said formulation, is provided at an amount of at least 100 mg/dose, or at least 150 mg/dose, or at least 200 mg/dose.

Embodiment 45: The formulation of embodiment 44, wherein the nicotinamide and/or nicotinamide analog, or nicotinamide metabolite, when present, is administered in an amount of about 200 mg/dose.

Embodiment 46: The formulation according to any one of embodiments 1-45, wherein the piperine or piperine analog, when present in said formulation, is provided at an amount of at least 5 mg/dose, or at least 10 mg/dose, or at least 15 mg/dose.

Embodiment 47: The formulation of embodiment 46, wherein the piperine or piperine analog, when present, is administered in an amount of about 15 mg/dose.

Embodiment 48: The formulation according to any one of embodiments 1-47, wherein the pyridoxine or pyridoxine analog, when present in said formulation, is provided at an amount of at least about 10 mg/dose, or at least about 25 mg/dose, or at least about 50 mg/dose.

Embodiment 49: The formulation of embodiment 48, wherein the pyridoxine or pyridoxine analog, when present is administered in an amount of about 50 mg/dose.

Embodiment 50: The formulation according to any one of embodiments 1-49, wherein the thiamine and/or a thiamine analog, when present in said formulation, is provided at an amount of at least 50 mg/dose, or at least 100 mg/dose.

Embodiment 51: The formulation of embodiment 50, wherein the thiamine and/or thiamine analog is administered in an amount of about 100 mg/dose.

Embodiment 52: The formulation of embodiment 1, wherein said formulation comprises:
alpha lipoic acid; nicotinamide;
thiamine provided as thiamine mononitrate (vitamin B1);
piperine; and
pyridoxamine provided as pyridoxine HCL.

Embodiment 53: The formulation of embodiment 52, wherein said formulation does not contain additional vitamins or dietary supplements.

Embodiment 54: The formulation according to any one of embodiments 52-53, wherein the metabolically active ingredients in said formulation consist of:
alpha lipoic acid;
nicotinamide;
thiamine provided as thiamine mononitrate (vitamin B1);
piperine; and
pyridoxamine provided as pyridoxine HCL.

Embodiment 55: The formulation according to any one of embodiments 52-54, wherein said alpha lipoic acid is present in an amount ranging from about 100 mg up to about 200 mg per unit formulation.

Embodiment 56: The formulation according to any one of embodiments 52-55, wherein said nicotinamide is present in an amount ranging from about 100 mg up to about 300 mg per unit formulation.

Embodiment 57: The formulation according to any one of embodiments 52-56, wherein said thiamine mononitrate is present in an amount ranging from about 50 mg up to about 200 mg per unit formulation.

Embodiment 58: The formulation according to any one of embodiments 52-57, wherein said pyridoxamine HCL is present in an amount ranging from about 25 mg up to about 100 mg per unit formulation.

Embodiment 59: The formulation according to any one of embodiments 52-58, wherein said piperine is present in an amount ranging from about 5 mg up to about 25 mg per unit formulation.

Embodiment 60: The formulation according to any one of embodiments 52-59, wherein a unit formulation comprises:
about 150 mg alpha lipoic acid;
about 200 mg nicotinamide;
about 100 mg thiamine mononitrate;
about 15 mg piperine; and
about 50 mg pyridoxine HCL.

Embodiment 61: The formulation according to any one of embodiments 52-60, wherein said formulation further comprises a binder.

Embodiment 62: The formulation of embodiment 61, wherein said binder comprises microcrystalline cellulose.

Embodiment 63: The formulation according to any one of embodiments 52-62, wherein said formulation further comprises a metallic salt (boundary lubricant).

Embodiment 64: The formulation of embodiment 63, wherein said metallic salt comprises magnesium stearate.

Embodiment 65: The formulation according to any one of embodiments 52-64, wherein said formulation further comprises silicon dioxide.

Embodiment 66: The formulation according to any one of embodiments 52-65, wherein a unit dosage formulation comprises a gelatin capsule.

Embodiment 67: A method for inducing or increasing weight loss or reducing or preventing weight gain in a mammal, said method comprising:

administering to said mammal an effective amount of a combination of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 68: The method of embodiment 67, wherein combination of agents in said formulation provides a synergistic effect in the induction or increase of weight loss or the reduction or prevention of weight gain.

Embodiment 69: The method according to any one of embodiments 67-68, wherein said method comprises a method of inducing or increasing weight loss.

Embodiment 70: The method according to any one of embodiments 67-68, wherein said method comprises reducing or preventing weight gain.

Embodiment 71: The method according to any one of embodiments 67-70, wherein said method reduces carbohydrate consumption by said mammal.

Embodiment 72: The method according to any one of embodiments 67-71, wherein said method does not substantially alter lipid consumption by said mammal.

Embodiment 73: The method according to any one of embodiments 67-72, wherein administering does not result in nausea.

Embodiment 74: The method according to any one of embodiments 67-73, wherein comprises a method for reducing blood glucose in said mammal.

Embodiment 75: The method according to any one of embodiments 67-74, wherein said method comprises a method for reducing A1C in said mammal.

Embodiment 76: The method according to any one of embodiments 67-75, wherein said method comprises a method for the treatment or prophylaxis of diabetes.

Embodiment 77: The method of embodiment 76, wherein said method increases the amount of insulin release in a mammal with diabetes or pre-diabetes, or restores the amount of insulin release in a mammal with diabetes or pre-diabetes to substantially normal levels.

Embodiment 78: A method of ameliorating one or more symptoms of an obesity related disease in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 79: The method of embodiment 78, wherein said obesity-related disease comprise one or more pathologies selected from the group consisting of nonalcoholic fatty liver disease (NAFLD), high blood pressure, high cholesterol, high blood sugar, heart disease, stroke, and obesity-related cancer.

Embodiment 80: The method of embodiment 79, wherein said obesity-related disease comprises NAFLD.

Embodiment 81: The method of embodiment 80, wherein said obesity-related disease comprises nonalcoholic steatohepatitis (NASH).

Embodiment 82: The method according to any one of embodiments 78-81, wherein said combination of agents provides a synergistic effect in ameliorating one or more symptoms or, and/or slowing or stopping the progression of, and/or to curing said obesity-related disease.

Embodiment 83: The method according to any one of embodiments 67-82, wherein said mammal is a mammal identified as having elevated triglycerides.

Embodiment 84: The method according to any one of embodiments 67-83, wherein said mammal is a mammal diagnosed as pre-diabetic.

Embodiment 85: The method according to any one of embodiments 67-84, wherein said mammal is a mammal diagnosed as having diabetes.

Embodiment 86: The method of embodiment 78, wherein said effective amount is an amount sufficient to ameliorate a complication of diabetes selected from the group consisting of diabetic neuropathy, cardiomyopathy, nephropathy, retinopathy, microvascular damage, and early mortality.

Embodiment 87: The method of embodiment 86, wherein said combination of agents provides a synergistic effect in ameliorating a complication of diabetes selected from the group consisting of diabetic neuropathy, cardiomyopathy, nephropathy, retinopathy, microvascular damage, and early mortality.

Embodiment 88: The method according to any one of embodiments 67-87, wherein said method, produces a reduction in one or more advanced glycation end products.

Embodiment 89: The method of embodiment 88, wherein said method produces a reduction in, or slows the accumulation of, glyoxal/GO.

Embodiment 90: The method according to any one of embodiments 88-89, wherein said method produces a reduction in, or slows the accumulation of, methylglyoxal/MGO.

Embodiment 91: The method according to any one of embodiments 88-90, wherein said method produces a reduction in, or slows the accumulation of 3-deoxyglucosone/3DG.

Embodiment 92: A method of providing neuroprotection to a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 93: The method of embodiment 92, wherein said neuroprotection slows the progression, stops the progression and/or ameliorates neuronal damage associated with a neurodegenerative disease.

Embodiment 94: The method of embodiment 93, wherein said neurodegenerative disease comprises a disease selected from the group consisting of Mild Cognitive Impairment (MCI), Alzheimer's disease, Parkinson's disease, amyotrophic lateral sclerosis, and Huntington's disease.

Embodiment 95: The method according to any one of embodiments 92-93, wherein said combination of agents provides a synergistic effect in slowing the progression, stopping the progression and/or ameliorating neuronal damage associated with said neurodegenerative disease.

Embodiment 96: A method of improving memory and/or cognition in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 97: The method of embodiment 96, wherein said mammal is a mammal with age-related diminution in cognition and/or age-related memory loss.

Embodiment 98: A method of improving muscle strength in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 99: The method of embodiment 98, wherein said mammal is a mammal with age-related muscle wasting (sarcopenia).

Embodiment 100: The method of embodiment 98, wherein said mammal is a mammal with disease-associated muscle wasting.

Embodiment 101: The method of embodiment 100, wherein said disease-associated muscle wasting is muscle wasting associated with a pathology selected from the group consisting of amyotrophic lateral sclerosis (ALS), muscular dystrophy (MD), multiple sclerosis (MS), and spinal muscular atrophy.

Embodiment 102: A method of reducing inflammation in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 103: A method of upregulating neurotrophic factors in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 104: The method of embodiment 103, wherein said neurotrophic factors comprises BDNF.

Embodiment 105: A method of increasing endurance, and/or improving muscle activity, and/or improving and muscle recovery in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 106: A method of improving cardiac fitness and/or reducing arterial stiffness in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

Embodiment 107: The method according to any one of embodiments 67-106, wherein said combination of agents comprises a combination of agents found in the formulation according to according to any one of embodiments 1-66.

Embodiment 108: The method according to any one of embodiments 67-106, wherein said method comprises administering to said mammal a formulation according to according to any one of embodiments 1-66.

Embodiment 109: The method according to any one of embodiments 67-106, wherein said combination of agents comprises lipoic acid or an analog thereof or a pharmaceutically acceptable salt of said lipoic acid or analog.

Embodiment 110: The method of embodiment 109, wherein said combination of agents comprises lipoic acid.

Embodiment 111: The method of embodiment 109, wherein said combination of agents comprises a lipoic acid analog.

Embodiment 112: The method of embodiment 111, wherein said lipoic acid analog comprises an analog selected from the group consisting of bisnor-lipoic (1,2-dithiolane-3-propanoic), and tetranorlipoic (1,2-dithiolane-3-carboxylic) acid, and a lipoic acid amide (e.g., 6,8-dithiooctanoic amide, 2-(N,N-dimethylamine) ethylamido lipoate, etc.).

Embodiment 113: The method according to any one of embodiments 67-112, wherein said combination of agents comprises nicotinamide or a nicotinamide analog or nicotinamide metabolite, or a pharmaceutically acceptable salt of said nicotinamide or nicotinamide analog or nicotinamide analyte.

Embodiment 114: The method of embodiment 113, wherein said nicotinamide or nicotinamide analog comprises nicotinamide.

Embodiment 115: The method of embodiment 113, wherein said nicotinamide or nicotinamide analog comprises a nicotinamide metabolite.

Embodiment 116: The method of embodiment 115, wherein said nicotinamide metabolite comprise nicotinamide mononucleotide (NMN) or nicotinamide ribonucleoside (NMR).

Embodiment 117: The method of embodiment 113, wherein said nicotinamide or nicotinamide analog comprises nicotinic acid (pyridine-3-carboxylic acid), nicotinamide (nicotinic acid amide), or inositol hexanicotinate).

Embodiment 118: The method of embodiment 113, wherein said nicotinamide or an analog thereof comprises a nicotinamide analog.

Embodiment 119: The method of embodiment 118, wherein said nicotinamide or an analog thereof comprises a nicotinamide analog nicotinamide analog shown in Table 1.

Embodiment 120: The method according to any one of embodiments 113-119, wherein said nicotinamide or nicotinamide analog is a substantially pure R enantiomer.

Embodiment 121: The method according to any one of embodiments 113-119, wherein said nicotinamide or nicotinamide analog is a substantially pure S enantiomer.

Embodiment 122: The method according to any one of embodiments 67-121, wherein said combination of agents comprises thiamine or a thiamine analog or a pharmaceutically acceptable salt of said thiamine or thiamine analog.

Embodiment 123: The method of embodiment 122, wherein said thiamine or thiamine analog comprises thiamine.

Embodiment 124: The method of embodiment 122, wherein said thiamine or thiamine analog comprises a thiamine analog.

Embodiment 125: The method of embodiment 124, wherein said thiamine analog comprises a thiamine analog selected from the group consisting of pyrithiamine, thiamine disulphide and acetylthiamine, oxythiamine, thiamine diphosphate, oxythiamine diphsophate, neopyrithiamine, 4-methyl-5-(hydroxymethyl)-thiazole, β-(4-Methylthiazolyl-5)-alanine, benfotiamine, and 3-(2, 4-dioxo-1, 2, 3, 4-tetrahydro-5-pyrimidinyl) methyl-4-methyl-5-(2-hydroxyethyl) thiazolium nitrate.

Embodiment 126: The method according to any one of embodiments 122-125, wherein said thiamine or thiamine analog comprise a substantially pure R enantiomer.

Embodiment 127: The method according to any one of embodiments 122-125, wherein said thiamine or thiamine analog comprise a substantially pure S enantiomer.

Embodiment 128: The method according to any one of embodiments 67-127, wherein said combination of agents comprises piperine and/or a piperine analog or a pharmaceutically acceptable salt of said comprises piperine and/or piperine analog analog.

Embodiment 129: The method of embodiment 128, wherein said piperine or piperine analog comprises piperine.

Embodiment 130: The method of embodiment 128, wherein said piperine or piperine analog comprises a piperine analog.

Embodiment 131: The method of embodiment 130, wherein said piperine analog comprises a piperine analog selected from the group consisting of 1-(3,4-methylenedioxyphenyl)-penta-2E,4E-dienoic acid methyl ester, 1-E,E-piperinoyl-isobutylamine, and 1-(3,4-methylenedioxyphenyl)-pentanoic acid cyclohexyl amide.

Embodiment 132: The method of embodiment 130, wherein said piperine analog comprises a piperine analog shown in Table 2.

Embodiment 133: The method of embodiment 130, wherein said piperine analog comprises a piperine analog selected from the group consisting of P1057, P622, P545, P725, P557, P2, P4, P1045, P1090, P8, P594, P1087, P1088, P28, P12, P1118, P1, P1122, P677, P1120, P1121, P6, P1112, P1119, P1117, P1116, P1070, P1114, P569, P32, P5, P1084, P1069, P593, P10, P9, P665, P7, P17, P1123, P11, P1080, P33, P743, P16, P1078, P27, P604, P25, P29, 1073, P26, P30, P707, P752, P22, P670, P23, P21, P546, P3, P581, P15, P636, P20, P737, P649, and P689.

Embodiment 134: The method according to any one of embodiments 128-133, wherein said piperine or piperine analog comprises a substantially pure R enantiomer.

Embodiment 135: The method according to any one of embodiments 128-133, wherein said piperine or piperine analog comprises a substantially pure S enantiomer.

Embodiment 136: The method according to any one of embodiments 67-135, wherein said combination of agents comprises pyridoxamine and/or a pyridoxamine analog or a pharmaceutically acceptable salt of said pyridoxamine or pyridoxamine analog.

Embodiment 137: The method of embodiment 136, wherein said pyridoxamine or a pyridoxamine analog comprises pyridoxamine.

Embodiment 138: The method of embodiment 136, wherein said pyridoxamine or a pyridoxamine analog comprises a pyridoxamine analog.

Embodiment 139: The method of embodiment 138, wherein said pyridoxamine analog comprises a pyridoxamine analog selected from the group consisting of salicylamine (o-hydroxybenzylamine), thiosalicylamine (o-mercaptobenzylamine), and 3-hydroxy-4-aminomethylpyridine, 3-hydroxy-4-aminomethylpyridine, 1-methylpyridoxamine chloride, 1-methyl-3-hydroxy-4-aminomethylpyridinium chloride, pyridoxamine phosphate, and an alkyl-pyridoxamine (alkyl-PM).

Embodiment 140: The method of embodiment 138, wherein said pyridoxamine analog comprises an alkyl pyridoxamine.

Embodiment 141: The method of embodiment 140, wherein said pyridoxamine analog comprises a pentyl-PM, a hexyl-PM, or a heptyl-PM.

Embodiment 142: The method according to any one of embodiments 136-141, wherein said pyridoxamine or pyridoxamine comprises a substantially pure R enantiomer.

Embodiment 143: The method according to any one of embodiments 136-141, wherein said pyridoxamine or pyridoxamine comprises a substantially pure S enantiomer.

Embodiment 144: The method according to any one of embodiments 67-143, wherein said agents comprising said combination of two or more agents comprises lipoic acid, thiamine, nicotinamide, piperine, and pyridoxamine in substantially equal amounts.

Embodiment 145: The method according to any one of embodiments 67-143, wherein said agents comprising said combination of two or more agents comprises lipoic acid, thiamine, nicotinamide, and pyridoxamine in substantially equal amounts, and piperidine in a greater amount the other four compounds.

Embodiment 146: The method according to any one of embodiments 67-145, wherein said agents comprising said combination of two or more agents are administered simultaneously.

Embodiment 147: The method of embodiment 146, wherein said combination of two or more agents are administered in a combined formulation.

Embodiment 148: The method according to any one of embodiments 67-145, wherein said agents comprising said combination of two of more agents are not administered simultaneously.

Embodiment 149: The method according to any one of embodiments 67-148, wherein said agents are administered via a route independently selected from the group consisting of oral delivery, isophoretic delivery, transdermal delivery, parenteral delivery, aerosol administration, administration via inhalation, intravenous administration, and rectal administration.

Embodiment 150: The method of embodiment 149, wherein said agents are orally administered to said mammal.

Embodiment 151: The method according to any one of embodiments 67-150, wherein said mammal is a human.

Embodiment 152: The method according to any one of embodiments 67-150, wherein said mammal is a non-human mammal.

Embodiment 153: The method according to any one of embodiments 67-152, wherein the lipoic acid and/or lipoic acid analog, when present is administered in an amount of at least 50 mg/dose, or at least 100 mg/dose, or at least 150 mg/dose.

Embodiment 154: The method of embodiment 153, wherein the lipoic acid and/or lipoic acid analog, when present is administered in an amount of about 150 mg/dose.

Embodiment 155: The method according to any one of embodiments 67-154, wherein the nicotinamide and/or nicotinamide analog, or nicotinamide metabolite, when present, is administered in an amount of at least 100 mg/dose, or at least 150 mg/dose, or at least 200 mg/dose.

Embodiment 156: The method of embodiment 155, wherein the nicotinamide and/or nicotinamide analog, or nicotinamide metabolite, when present, is administered in an amount of about 200 mg/dose.

Embodiment 157: The method according to any one of embodiments 67-156, wherein the piperine or piperine analog, when present, is administered in an amount of at least 5 mg/dose, or at least 10 mg/dose, or at least 15 mg/dose.

Embodiment 158: The method of embodiment 157, wherein the piperine or piperine analog, when present, is administered in an amount of about 15 mg/dose.

Embodiment 159: The method according to any one of embodiments 67-158, wherein the pyridoxine or pyridoxine analog, when present is administered in an amount of at least about 10 mg/dose, or at least about 25 mg/dose, or at least about 50 mg/dose.

Embodiment 160: The method of embodiment 159, wherein the pyridoxine or pyridoxine analog, when present is administered in an amount of about 50 mg/dose.

Embodiment 161: The method according to any one of embodiments 67-160, wherein the thiamine and/or a thiamine analog, when present, is administered in an amount of at least 50 mg/dose, or at least 100 mg/dose.

Embodiment 162: The method of embodiment 161, wherein the thiamine and/or thiamine analog is administered in an amount of about 100 mg/dose.

Embodiment 163: The method according to any one of embodiments 67-162, wherein said agents are administered once a day, or twice a day, or 3 times/day, or 4 times/day.

Embodiment 164: A method of inducing or increasing feeding and weight gain in a mammal, said method comprising: administering to said mammal an effective amount of hydroimidazolone N_{δ}-(5-hydro-5-methyl-4-imidazolon-2-yl) ornithine (MG-H1).

Embodiment 165: The method of embodiment 164, wherein said mammal is a mammal that has a pathology characterized by abnormal weight loss.

Embodiment 166: The method of embodiment 165, wherein said pathology is selected from the group consisting of dysphagia, painful mouth sores, newly applied orthodontic appliances, or loss of teeth, pyloric stenosis, hiatus hernia, coeliac disease, chronic pancreatitis, Crohn's disease, gastrointestinal infection, gastrointestinal fistulas, carcinoid disorders, intestinal hypermotility, hepatobiliary disease, food intolerance, medication induced weight loss, hyperthyroidism, Addison's disease, cancer (e.g., lymphoma, leukemia, carcinoma, sarcoma), heart failure, chronic respiratory disease, chronic kidney disease, liver failure, rheumatoid arthritis, systemic lupus erythematosus, acute infection, chronic infections (e.g., tuberculosis, HIV, parasitic infections, etc.), drug abuse, heavy smoking, stress-induced weight loss, depression, anorexia nervosa, food phobias, and Parkinson's disease.

Embodiment 167: The method according to any one of embodiments 164-166, wherein said hydroimidazolone N_{δ}-(5-hydro-5-methyl-4-imidazolon-2-yl) ornithine (MG-H1) is administered via a route selected from the group consisting of oral delivery, isophoretic delivery, transdermal delivery, parenteral delivery, aerosol administration, administration via inhalation, intravenous administration, and rectal administration.

Embodiment 168: The method of embodiment 167, wherein said MG-H1 is orally administered to said mammal.

Embodiment 169: The method according to any one of embodiments 164-168, wherein said mammal is a human.

Embodiment 170: The method according to any one of embodiments 164-168, wherein said mammal is a non-human mammal.

Embodiment 171: A pharmaceutical formulation comprising:

hydroimidazolone N_{δ}-(5-hydro-5-methyl-4-imidazolon-2-yl) ornithine (MG-H1); and a pharmaceutically acceptable carrier.

Embodiment 172: The formulation of embodiment 171, wherein said formulation is formulated for administration by a route selected from the group consisting of oral delivery, isophoretic delivery, transdermal delivery, parenteral delivery, aerosol administration, administration via inhalation, intravenous administration, and rectal administration.

Embodiment 173: The formulation of embodiment 172, wherein said formulation is compounded for oral administration.

Embodiment 174: The formulation of embodiment 172, wherein said formulation is sterile.

Embodiment 175: The formulation according to any one of embodiments 171-174, wherein said formulation is a unit dosage formulation.

Embodiment 176: A kit for inducing or increasing weight loss or reducing or preventing weight gain in a mammal, said kit comprising:
at least two agents selected from the group consisting of lipoic acid and/or a lipoic acid analog, nicotinamide and/or a nicotinamide analog nicotinamide or metabolite, thiamine and/or a thiamine analog, piperine and/or a piperine analog, pyridoxamine and/or a pyridoxamine analog.

Embodiment 177: The kit of embodiment 176, wherein each of said agents are provided in separate containers.

Embodiment 178: The kit of embodiment 176, wherein at least two of said agents are in the same container.

Embodiment 179: The kit of embodiment 176, wherein at least three of said agents are in the same container.

Embodiment 180: The kit of embodiment 176, wherein at least four of said agents are in the same container.

Embodiment 181: The kit of embodiment 176, wherein at least five of said agents are in the same container.

Embodiment 182: The kit according to any one of embodiments 176-181, wherein said combination of agents comprises a combination of agents found in the formulation according to according to any one of embodiments 1-66.

Embodiment 183: The kit of embodiment 176, wherein said kit comprises a container containing a formulation according to according to any one of embodiments 1-66.

Embodiment 184: The kit according to any one of embodiments 176-181, wherein said combination of agents comprises lipoic acid or an analog thereof or a pharmaceutically acceptable salt of said lipoic acid or analog.

Embodiment 185: The kit of embodiment 184, wherein said combination of agents comprises lipoic acid.

Embodiment 186: The kit of embodiment 184, wherein said combination of agents comprises a lipoic acid analog.

Embodiment 187: The kit of embodiment 186, wherein said lipoic acid analog comprises an analog selected from the group consisting of bisnor-lipoic (1,2-dithiolane-3-propanoic), and tetranorlipoic (1,2-dithiolane-3-carboxylic) acid, and a lipoic acid amide (e.g., 6,8-dithiooctanoic amide, 2-(N,N-dimethylamine) ethylamido lipoate, etc.).

Embodiment 188: The kit according to any one of embodiments 184-187, wherein said lipoic acid or lipoic acid analog comprises a substantially pure S enantiomer.

Embodiment 189: The kit according to any one of embodiments 184-187, wherein said lipoic acid or lipoic acid analog comprises a substantially pure R enantiomer.

Embodiment 190: The kit according to any one of embodiments 176-189, wherein said combination of agents comprises nicotinamide or a nicotinamide analog or nicotinamide metabolite or a pharmaceutically acceptable salt of said nicotinamide or nicotinamide analog or nicotinamide metabolite.

Embodiment 191: The kit of embodiment 190, wherein said nicotinamide or nicotinamide analog comprises nicotinamide.

Embodiment 192: The kit of embodiment 190, wherein said nicotinamide or nicotinamide analog comprises nicotinic acid (pyridine-3-carboxylic acid), nicotinamide (nicotinic acid amide), or inositol hexanicotinate).

Embodiment 193: The kit of embodiment 190, wherein said nicotinamide or nicotinamide analog comprises a nicotinamide analog.

Embodiment 194: The kit of embodiment 193, wherein said nicotinamide or nicotinamide analog comprises a nicotinamide analog nicotinamide analog shown in Table 1.

Embodiment 195: The kit according to any one of embodiments 190-194, wherein said nicotinamide or nicotinamide analog comprises a substantially pure R enantiomer.

Embodiment 196: The kit according to any one of embodiments 190-194, wherein said nicotinamide or nicotinamide analog comprises a substantially pure S enantiomer.

Embodiment 197: The kit according to any one of embodiments 176-196, wherein said combination of agents comprises thiamine or a thiamine analog or a pharmaceutically acceptable salt of said thiamine or thiamine analog.

Embodiment 198: The kit of embodiment 197, wherein said thiamine or a thiamine analog comprises thiamine.

Embodiment 199: The kit of embodiment 197, wherein said thiamine or a thiamine analog comprises a thiamine analog.

Embodiment 200: The kit of embodiment 197, wherein said thiamine analog comprises a thiamine analog selected from the group consisting of pyrithiamine, thiamine disulphide and acetylthiamine, oxythiamine, thiamine diphosphate, oxythiamine diphsophate, neopyrithiamine, 4-methyl-5-(hydroxymethyl)-thiazole, β-(4-Methylthiazolyl-5)-alanine, and 3-(2, 4-dioxo-1, 2, 3, 4-tetrahydro-5-pyrimidinyl) methyl-4-methyl-5-(2-hydroxyethyl) thiazolium nitrate.

Embodiment 201: The kit according to any one of embodiments 197-200, wherein said thiamine or a thiamine analog comprises a substantially pure R enantiomer.

Embodiment 202: The kit according to any one of embodiments 197-200, wherein said thiamine or a thiamine analog comprises a substantially pure S enantiomer.

Embodiment 203: The kit according to any one of embodiments 176-202, wherein said combination of agents comprises piperine and/or a piperine analog or a pharmaceutically acceptable salt of said piperine and/or piperine analog.

Embodiment 204: The kit of embodiment 203, wherein said piperine or piperine analog comprises piperine.

Embodiment 205: The kit of embodiment 203, wherein said piperine or piperine analog comprises a piperine analog.

Embodiment 206: The kit of embodiment 205, wherein said piperine analog comprises a piperine analog selected from the group consisting of 1-(3,4-methylenedioxyphenyl)-penta-2E,4E-dienoic acid methyl ester, 1-E,E-piperinoyl-isobutylamine, and 1-(3,4-methylenedioxyphenyl)-pentanoic acid cyclohexyl amide.

Embodiment 207: The kit of embodiment 205, wherein said piperine analog comprises a piperine analog shown in Table 2.

Embodiment 208: The kit of embodiment 205, wherein said piperine analog comprises a piperine analog selected from the group consisting of P1057, P622, P545, P725, P557, P2, P4, P1045, P1090, P8, P594, P1087, P1088, P28, P12, P1118, P1, P1122, P677, P1120, P1121, P6, P1112, P1119, P1117, P1116, P1070, P1114, P569, P32, P5, P1084, P1069, P593, P10, P9, P665, P7, P17, P1123, P11, P1080, P33, P743, P16, P1078, P27, P604, P25, P29, 1073, P26, P30, P707, P752, P22, P670, P23, P21, P546, P3, P581, P15, P636, P20, P737, P649, and P689.

Embodiment 209: The kit according to any one of embodiments 203-208, wherein said piperine or piperine analog comprises a substantially pure R enantiomer.

Embodiment 210: The kit according to any one of embodiments 203-208, wherein said piperine or piperine analog comprises a substantially pure S enantiomer.

Embodiment 211: The kit according to any one of embodiments 176-210, wherein said combination of agents comprises pyridoxamine and/or a pyridoxamine analog or a pharmaceutically acceptable salt of said pyridoxamine or pyridoxamine analog.

Embodiment 212: The kit of embodiment 211, wherein said pyridoxamine or pyridoxamine analog comprises pyridoxamine.

Embodiment 213: The kit of embodiment 211, wherein said pyridoxamine or pyridoxamine analog comprises a pyridoxamine analog.

Embodiment 214: The kit of embodiment 213, wherein said pyridoxamine analog comprises a pyridoxamine analog selected from the group consisting of salicylamine (o-hydroxybenzylamine), thiosalicylamine (o-mercaptobenzylamine), and 3-hydroxy-4-aminomethylpyridine, 3-hydroxy-4-aminomethylpyridine, 1-methylpyridoxamine chloride, 1-methyl-3-hydroxy-4-aminomethylpyridinium chloride, pyridoxamine phosphate, and an alkyl-pyridoxamine (alkyl-PM).

Embodiment 215: The kit of embodiment 213, wherein said pyridoxamine analog comprises an alkyl pyridoxamine.

Embodiment 216: The kit of embodiment 215, wherein said pyridoxamine analog comprises a pentyl-PM, a hexyl-PM, or a heptyl-PM.

Embodiment 217: The kit according to any one of embodiments 211-216, wherein said pyridoxamine or pyridoxamine analog comprises a substantially pure R enantiomer.

Embodiment 218: The kit according to any one of embodiments 211-216, wherein said pyridoxamine or pyridoxamine analog comprises a substantially pure S enantiomer.

Embodiment 219: The kit according to any one of embodiments 176-218, wherein said agents comprising said two or more agents comprise lipoic acid, thiamine, nicotinamide, piperine, and pyridoxamine in substantially equal amounts.

Embodiment 220: The kit according to any one of embodiments 176-218, wherein said agents comprising said two or more agents comprise lipoic acid, thiamine, nicotinamide, and pyridoxamine in substantially equal amounts, and piperidine in a greater amount the other four compounds.

Embodiment 221: The methods and/or formulations, and/or kits according to embodiments 1-220, wherein the agents used in said methods, formulations or kits expressly exclude one or more agents selected from the group consisting of vitamin C, benfotiamine, pyridoxamine, alpha-lipoic acid, taurine, pimagedine, aspirin, carnosine, metformin, pioglitazone, pentoxifylline, resveratrol, and curcumin.

Embodiment 222: The methods and/or formulations, and/or kits according to embodiments 1-221, wherein the agents used in said methods, formulations or kits expressly exclude active agents other than lipoic acid and/or a lipoic acid analog, nicotinamide and/or a nicotinamide analog, thiamine and/or a thiamine analog, piperine and/or a piperine analog, pyridoxamine and/or a pyridoxamine analog.

Embodiment 223: The methods and/or formulations, and/or kits according to embodiments 1-222, wherein the agents used in said methods, formulations or kits expressly are not components of a multivitamin containing additional vitamins.

### Definitions

Unless otherwise indicated, reference to a compound (e.g., lipoic acid and/or a lipoic acid analog, and/or nicotinamide and/or a nicotinamide analog, and/or thiamine, and/or a thiamine analog, and/or piperine, and/or a piperine analog, and/or pyridoxamine, and/or a pyridoxamine analog described herein) should be construed broadly to include pharmaceutically acceptable salts, prodrugs, tautomers, alternate solid forms, non-covalent complexes, and combinations thereof.

Generally, reference to a certain element such as hydrogen or H is meant to include all isotopes of that element. For example, if an R group is defined to include hydrogen or H, it also includes deuterium and tritium. Accordingly, isotopically labeled compounds are within the scope of this invention.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counterions. Salts can form from or incorporate one or more deprotonated acidic groups (e.g., carboxylic acids), one or more protonated basic groups (e.g., amines), or both (e.g. zwitterions).

A prodrug is a compound that is converted to a therapeutically active compound after administration. For example, conversion may occur by hydrolysis of an ester group, such as a C₁-C₆ alkyl ester of the carboxylic acid group of the present compounds, or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in equilibrium with one another. For example, tautomers may be related by transfer of a proton, hydrogen atom, or hydride ion.

Unless stereochemistry is explicitly depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like. In various embodiments alternate solid forms of any of the compounds described herein are contemplated.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN), and the like.

The term “alkyl” refers to and covers any and all groups that are known as normal alkyl, branched-chain alkyl, cycloalkyl and also cycloalkyl-alkyl. Illustrative alkyl groups include, but are not limited to methyl, ethyl, n-propyl, isopropyl, n-butyl, isobutyl, sec-butyl, t-butyl, octyl, and decyl. The term “cycloalkyl” refers to cyclic, including polycyclic, saturated hydrocarbyl groups. Examples include, but are not limited to cyclopentyl, cyclohexyl, dicyclopentyl, norbornyl, octahydronapthyl, and spiro[3.4]octyl. In certain embodiments, alkyl groups contain 1-12 carbon atoms (C₁₋₁₂ alkyl), or 1-9 carbon atoms (C₁₋₉ alkyl), or 1-6 carbon atoms (C₁₋₆ alkyl), or 1-5 carbon atoms (C₁₋₅ alkyl), or carbon atoms (C₁₋₄ alkyl), or 1-3 carbon atoms (C₁₋₃ alkyl), or 1-2 carbon atoms (C₁₋₂ alkyl).

By way of example, the term “C₁₋₆ alkyl group” refers to a straight chain or branched chain alkyl group having 1 to 6 carbon atoms, and may be exemplified by a methyl group, an ethyl group, an n-propyl group, an isopropyl group, an n-butyl group, an isobutyl group, a tert-butyl group, a sec-butyl group, an n-pentyl group, a tert-amyl group, a 3-methylbutyl group, a neopentyl group, and an n-hexyl group.

The term “alkoxy” as used herein means an alkyl group bound through a single, terminal oxygen atom. An “alkoxy” group may be represented as —O-alkyl where alkyl is as defined above. The term “aryloxy” is used in a similar fashion, and may be represented as —O-aryl, with aryl as defined below. The term “hydroxy” refers to —OH.

Similarly, the term “alkylthio” as used herein means an alkyl group bound through a single, terminal sulfur atom. An “alkylthio” group may be represented as —S-alkyl where alkyl is as defined above. The term “arylthio” is used similarly, and may be represented as —S-aryl, with aryl as defined below. The term “mercapto” refers to —SH.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

The term “heteroaryl group” refers to a monocyclic or condensed-ring aromatic heterocyclic group containing one or more hetero-atoms selected from O, S and N. If the aromatic heterocyclic group has a condensed ring, it can include a partially hydrogenated monocyclic group. Examples of such a heteroaryl group include a pyrazolyl group, a thiazolyl group, an isothiazolyl group, a thiadiazolyl group, an imidazolyl group, a furyl group, a thienyl group, an oxazolyl group, an isoxazolyl group, a pyrrolyl group, an imidazolyl group, a (1,2,3)- and (1,2,4)-triazolyl group, a tetrazolyl group, a pyranyl group, a pyridyl group, a pyrimidinyl group, a pyrazinyl group, a pyridazinyl group, a quinolyl group, an isoquinolyl group, a benzofuranyl group, an isobenzofuranyl group, an indolyl group, an isoindolyl group, an indazolyl group, a benzoimidazolyl group, a benzotriazolyl group, a benzoxazolyl group, a benzothiazolyl group, a benzo[b]thiophenyl group, a thieno[2,3-b]thiophenyl group, a (1,2)- and (1,3)-benzoxathiol group, a chromenyl group, a 2-oxochromenyl group, a benzothiadiazolyl group, a quinolizinyl group, a phthalazinyl group, a naphthyridinyl group, a quinoxalinyl group, a quinazolinyl group, a cinnolinyl group, and a carbazolyl group.

A “derivative” of a compound means a chemically modified compound wherein the chemical modification takes place at one or more functional groups of the compound. The derivative however, is expected to retain, or enhance, the pharmacological activity of the compound from which it is derived.

As used herein, “administering” refers to local and systemic administration, e.g., including enteral, parenteral, pulmonary, and topical/transdermal administration. Routes of administration for agents (e.g., TRPA1 activator(s) described herein, or a tautomer(s) or stereoisomer(s) thereof, or pharmaceutically acceptable salts or solvates of said activator(s), said stereoisomer(s), or said tautomer(s), or analogues, derivatives, or prodrugs thereof) that find use in the methods described herein include, e.g., oral (per os (p.o.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering the agent(s) described herein or composition to a mammal so that the agent(s) or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “effective amount” or “pharmaceutically effective amount” refers to the amount and/or dosage, and/or dosage regime of one or more agent(s) necessary to bring about the desired result e.g., an amount sufficient to ameliorate one or more symptoms of the pathology (e.g., a pathology characterized by advanced glycation end products as described herein and/or to slow or stop the onset of the pathology, and/or to lower the level of α-dicarbonyl compounds, and so forth. Such pathologies include but are not limited to diabetes or a complication thereof.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to reduction or elimination of one or more symptoms of a pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, but is not limited to, reduction or elimination of one or more markers that are characteristic of the pathology or disease (e.g., AGE levels).

As used herein, the phrase “consisting essentially of” refers to the genera or species of active pharmaceutical agents recited in a method or composition, and further can include other agents that, on their own do not offer or alter substantial activity for the recited indication or purpose. In various embodiments the activity comprises an activity provided by a combination of lipoic acid and/or a lipoic acid analog, nicotinamide and/or a nicotinamide analog or nicotinamide metabolite, thiamine and/or a thiamine analog, piperine and/or a piperine analog, and pyridoxamine and/or a pyridoxamine analog.

The terms “subject”, “individual”, and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments the subject may not be under the care or prescription of a physician or other health worker.

The term “formulation” or “drug formulation” or “dosage form” or “pharmaceutical formulation” as used herein refers to a composition containing at least one therapeutic agent or medication for delivery to a subject. In certain embodiments the dosage form comprises a given “formulation” or “drug formulation” and may be administered to a patient in the form of a lozenge, pill, tablet, capsule, suppository, membrane, strip, liquid, patch, film, gel, spray or other form.

The term “substantially pure” means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical or chemical properties, of the compound. Methods for purification of the compounds to produce substantially chemically pure compounds are known to those of skill in the art. A substantially chemically pure compound may, however, be a mixture of stereoisomers or isomers. In such instances, further purification might increase the specific activity of the compound.

The term “substantially pure” when used with respect to enantiomers indicates that one particular enantiomer (e.g. an S enantiomer or an R enantiomer) is substantially free of its stereoisomer. In various embodiments substantially pure indicates that a particular enantiomer is at least 70%, or at least 80%, or at least 90%, or at least 95%, or at least 98%, or at least 99% of the purified compound. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr. 113(3): 283-302). Racemic mixtures of chiral compounds of the can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

The term “Gly-low” refers to a cocktail of anti-AGEs compounds as described herein. In certain embodiments Gly-low comprises a combination of lipoic acid, nicotinamide, thiamine, piperine, and pyridoxamine. In certain embodiments Gly-low is administered by administering the constituent AGE compounds separately. In certain embodiments Gly-low is provided as a combined formulation comprising lipoic acid, nicotinamide, thiamine, piperine, and pyridoxamine.

The term “unit dosage” and “unit dosage form” are used interchangeably to refer to the form of pharmaceutical drug products or dietary supplements in the form in which they are provided (e.g., marketed for use). In certain embodiments the unit dosage form includes a specific mixture of active ingredients and inactive component s (excipients), in a particular configuration (such as a capsule shell, for example), and apportioned into a particular dose. The term unit dose can also sometimes encompass non-reusable packaging as well (especially when each drug product is individually packaged), although the FDA distinguishes that by unit-dose “packaging” or “dispensing”. Depending on the context, multi(ple) unit dose can refer to distinct drug products packaged together, or to a single drug product containing multiple drugs and/or doses. The term dosage form can also sometimes refer only to the pharmaceutical formulation of a drug product's constituent drug substance(s) and any blends involved, without considering matters beyond that (like the manner in which it is ultimately configured as a consumable product such as a capsule, patch, etc.). Depending on the method/route of administration, dosage forms come in several types. These include, but are not limited to many kinds of liquid, solid, and semisolid dosage forms. Common dosage forms include pill, tablet, or capsule, drink or syrup, and natural or herbal form such as plant or food of sorts, among many others. Notably, the route of administration (ROA) for drug delivery is dependent on the dosage form of the substance in question. A liquid dosage form is the liquid form of a dose of a chemical compound used as a drug or medication intended for administration or consumption. Various dosage forms may exist for a single particular active agent or combination thereof, since different medical conditions can warrant different routes of administration. For example, persistent nausea, especially with vomiting, may make it difficult to use an oral dosage form, and in such a case, it may be necessary to use an alternative route such as inhalational, buccal, sublingual, nasal, suppository or parenteral instead. Additionally, a specific dosage form may be a requirement for certain kinds of drugs or dietary supplements, as there may be issues with various factors like chemical stability or pharmacokinetics.

The phrase “cause to be administered” refers to the actions taken by a medical professional (e.g., a physician), or a person prescribing and/or controlling medical care of a subject, that control and/or determine, and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like. It will be recognized that where “administering” is provided in the methods described herein, “causing to be administered” is also provided.

### BRIEF DESCRIPTION OF THE DRAWINGS

FIG. 1 illustrates the structures of piperidine, pyridoxamine, lipoic acid, nicotinamide, niacin, and thiamine.

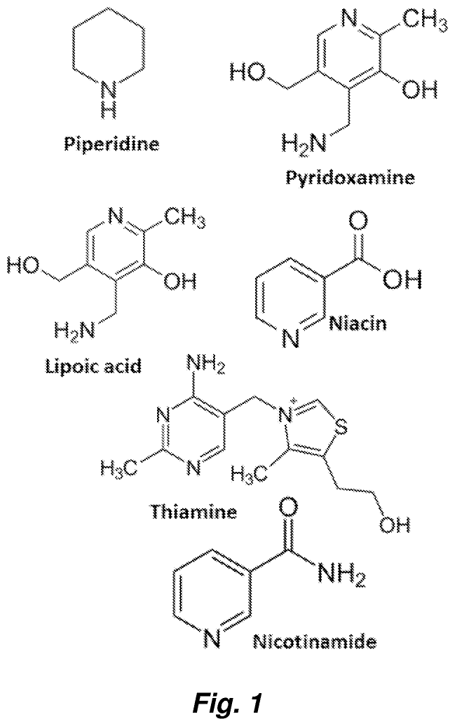

FIG. 2 shows that glucose enhances MGO levels in WT (N2) and more so in glod-4 animals. (Left) MGO levels in N2 and glod-4 mutants was measured with and without 2% glucose using and LC-MS/MS assay (Chaudhuri et al. (2016) Curr. Biol. 26: 3014-3025). (P<0.001).

FIG. 3. shows that the C. elegans glod-4 mutant that accumulates MGO has an enhanced feeding rate, especially when glucose is added to the media. Pharyngeal pumping was visually counted in worms treated with 2% glucose for 24 h as young adults. Statistics: One-way ANOVA; ** p<0.01 and **** p<0.0001. ns—not significant.

FIG. 4 illustrates a proposed model for how MGO is sensed by TRPA1 to activate SKN-1/Nrf2 and mediate detoxification of MGO through downstream glyoxalases in worms and mammals. MGO is sensed by TRPA-1 leading to the activation of SKN-1/Nrf2 pathway. This in turn activates downstream glyoxalases GLO1 and DJ1 (Id.).

FIG. 5 shows the identification of a combination of GRAS compounds that rescue MGO induced neural damage by lowering AGEs. A five-compound cocktail including ALA compared to other single, double, triple or ternary combinations was most effective at lowering MGO toxicity in N27 and SY-SH5Y neurons. Dose response curves for neurite length relative to 125 μM MGO treated neurons. The drug concentrations range from 1 nM to 100 μM total compound. Arrows refer to the neurite length in normal vs damaged neurons. The five-compound mix was significantly different from the other sets of compounds (P<0.01).

FIG. 6 shows that anti-AGEs compounds reduce body weight in db/db mice in a dose dependent manner. Body weight was monitored once weekly, in a longitudinal study of db/db mice on a regular chow diet (n=18), or varying concentrations of an anti-AGEs supplemented diet (n=12). Significant difference (p<0.01) between control and 1× anti-AGEs (triangle) was observed.

FIG. 7 shows that anti-AGEs compounds reduce food consumption in db/db mice in a dose dependent manner. A longitudinal study of db/db mice on a regular chow diet (n=18), or anti-AGEs diet (n=12). Total cage food consumption was monitored once weekly by weight and corrected by number of animals per cage. Significant difference (p<0.05) between control and 1× anti-AGEs (red triangle) was observed.

FIG. 8 shows that anti-AGEs treatment reduces oxygen consumption and energy expenditure in db/db mice. Anti-AGEs treated db/db mice underwent 4 days of metabolic cage testing, where oxygen consumption (top) and energy expenditure rates (bottom) were tracked every five minutes. Day readings pertain to values collected between 0600 and 1800 hours, whereas night readings pertain to values collected between 1800 and 0600 hours. (**p<0.005, ***p<0.0005).

FIG. 9 shows that anti-AGEs compounds reduced fat mass and maintained lean mass in db/db mice. Average lean mass percentage (Top) and average fat mass percentage (Bottom) in control and db/db mice. Mice were subjected to EchoMRI body composition testing once a month. *** (p<0.0005)

FIG. 10 shows that anti-AGEs compounds help maintain a reduction in fasting blood glucose levels and insulin in db/db mice. (Top) Blood glucose levels of db/db mice reared on a regular chow diet or anti-AGEs supplemented diet. F-fasted and NF-Non-Fasted. (Bottom) Serum insulin levels of db/db mice reared on a regular chow diet or an anti-AGEs supplemented diet under non-fasted conditions. Blood glucose levels and serum insulin were checked after 14 weeks of the diet. ***p<0.0005)

FIG. 11, panels A-B, shows that anti-AGEs compounds (e.g., GLYLO™) improves survival and reduces MGO and MG-H1, an MGO derived AGE. Panel A) Anti-AGEs cocktail (Gly-low) treatment extends survival in the leptin receptor deficient db/db mice. Eight week old male db/db mice (n=12) and controls (n=17) were treated with Gly-low in the diet and observed for survival. Panel B) Gly-low compound treatment reduced Methylglyoxal (MGO) and AGEs MG-H1 in leptin receptor deficient db/db mice. Urine samples were collected and analyzed for methylglyoxal and MG-H1 using mass spectrometry. MGO was compared with the internal standard and MG-H1 modification is normalized with concentration of Leucine in the sample, Leucine is used as an internal control because it undergoes very minimal or no modifications. Each sample had n=5 and p<0.05.

FIG. 12 shows that anti-AGEs supplement diet causes a decrease in food consumption in a normal chow diet but not in a high fat diet. Food consumption rates were collected during a 5 day metabolic cage where anti-AGEs were given to C57/BL6 mice on normal chow (64% carbohydrate, 17% fat) or high fat diet (21% carbohydrate, 60% fat) Food consumption was calculated per 24 hours. ** (P<0.005)

FIG. 13 shows that oral administration of MG-H1 increase oxygen consumption and energy expenditure in C57BL/6 mice. Wildtype control mice receiving an oral administration of exogenous AGEs in their drinking water showed increased change in body weights and food consumption in a two-week period. Percent weight increases indicate the percent change from starting body weights to the weights at the end of the two-week diet. Daily food consumption was measured twice weekly. (***p<0.005).

FIG. 14 shows that co-staining of MG-H1 with vimentin suggests its localization in tanycytes. Representative hypothalamic sections stained for MG-H1 (red) showing lack of co-localization with Iba1+ (green) microglial cells (not shown) but co-localization with Vimentin+ (green) tanycytes. Scale bar: 50 uM; 3V, third ventricle.

FIG. 15 shows that an anti-AGEs supplemented diet reduces energy expenditure and oxygen consumption on a high carbohydrate containing normal chow diet but not a high fat diet. Anti-AGEs treated C57/BL6 mice on normal chow (64% carbohydrate, 17% fat) or high fat diet (21% carbohydrate, 60% fat) underwent 4 days of metabolic cage testing, where energy expenditure rates (top) and oxygen consumption (bottom) were tracked every five minutes. Day readings (0600 and 1800 hours), and night readings (1800 to 0600 hours). (**p<0.05, ***p<0.0005).

FIG. 16 shows that fasted mice did not show an aversion to feeding on an anti-AGEs supplemented diet. C57/BL6 Mice were singly housed and fasted for 18 hours and re-introduced to control or an anti-AGEs supplemented diet in normal chow (top) or high fat diet (HFD) (bottom). No significant change was observed in food intake on normal chow for the first 6 hours or on the high fat diet suggesting that aversion to anti-AGEs is not a likely reason for the reduced food intake. Food consumption rates were collected 2, 6, 12, and 24 hours post re-introduction to food in 4 mice under each condition. ** p<0.005, ***p<0.0005.

FIG. 17 illustrates a volcano plot showing changes in RNA expression in the hypothalamus after 24 week treatment of anti-AGEs compounds in db/db mice.

FIG. 18. db/db mice reared on an anti-AGEs supplemented chow diet showed quicker response times to a hotplate. A longitudinal study of hot plate reaction times of db/db mice untreated (n=18) and treated with an anti-AGEs compound treatment (n=12). Mice were subjected to a 52 C analgesia meter hot plate once a month. Top) average reaction times, including the baseline reading, between treatment groups. Bottom) average reaction times, excluding the baseline reading, between treatment groups. (*=p<0.05, **=p<0.005, ***=p<0.0005)

FIG. 19. db/db mice reared on an anti-AGEs supplemented chow diet showed improved heart health parameters collected during echocardiography. Mice were subjected to echocardiography at the beginning of their experiment, the middle, and at the end. Untreated db/db control mice are compared side by side with mice treated with an anti-AGEs compound cocktail supplemented diet.

FIG. 20, panels A-D, shows liver pathology reports of db/db mice both untreated and treated with an orally administered anti-AGEs compound cocktail. Panel A) 200× microscopy image of a hematoxylin and eosin (H&E) staining of liver samples of untreated and anti-AGEs treated db/db mice for gross pathology reports. Panel B) 200× microscopy image of trichrome staining of liver samples of untreated and anti-AGEs treated db/db mice for gross pathology reports. Panel C) Graphed scoring reports comparing SAF steatosis and SAF parameter totals between untreated and treated db/db mice. Panel D) Graphed scoring reports comparing NAS steatosis and NAS parameter totals between untreated and treated db/db mice.

FIG. 21, panels A-E, shows that Gly-low treatment enhances survival, reduces food consumption and improves health in 24 months old mice: Panel A) When started treatment at the age of 24 months Gly-low improved the lifespan of aged mice (p<0.016). Panel B) Treatment with Gly-low significantly reduced food intake in aged mice, n=20 (P<0.01). Panel C) In 24 months old mice treated with Gly-low treatment for 4 months (treatment started at 20 months) we observed increased muscular strength and co-ordination in rotarod test n=17-20 per group (p<0.05). Panel D) Gly low treatment also improved pulse wave velocity that measures the stiffness of arteries and considered as independent measure of cardiac health. 4 months on Gly-low significantly reduced the pulse wave velocity, n=17-20 (p<0.015). Panel E) C57 mice treated with Gly-low for 7 months displayed significant improvement in Glucose utilization n=9 per group (p<0.0001). In GTT assay mice were fasted overnight and injected with 2 g/kg body weight of a glucose solution.

FIG. 22 shows that Gly-low enhances the expression of genes involved in detoxification of AGEs in the hypothalamus. Glo1 (glyloxalasel), Hagh (hydroxyacyl glutathione), Gss (glutathione synthase), Park7 (Parkinson disease autosomal recessive gene 7, Aldh2 (aldehyde dehydrogenase 2), Adh1a1 (aldehyde dehydrogenase family A1), Akr1a1 (aldo-keto reductase member A1), Akr1b1 (aldo-keto reductase member B1), Akr7a5 (aldo-keto reductase family 7 member A5), Nfe2l2 (nuclear factor erythroid derived 2 like 2).

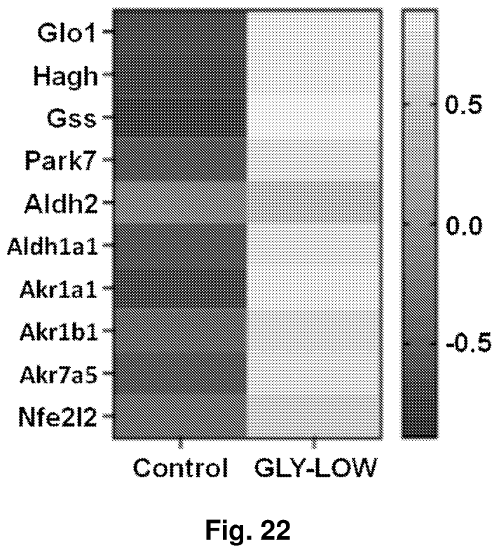

FIG. 23 shows that a Gly-low supplement diet causes a decrease in food consumption in a normal chow diet but not in a high fat diet. Food consumption rates were collected during a five day metabolic cage where anti-AGEs (Gly-low) were given to C57/BL6 mice on normal chow (60% carbohydrate, 21% fat) or high fat diet (21% carbohydrate, 60% fat). Food consumption was calculated per 24 hours. ** (P<0.005).

FIG. 24 shows that fasted mice did not show an aversion to feeding on an anti-AGEs supplemented diet. C57/BL6 Mice were singly housed and fasted for 18 hours and re-introduced to control or a Gly-low supplemented diet in normal chow (top) or high fat diet (HFD) (bottom). No significant change was observed in food intake on normal chow for the first 6 hours or on the high fat diet suggesting that aversion to anti-AGEs is not a likely reason for the reduced food intake. Food consumption rates were collected 2, 6, 12, and 24 hours post re-introduction to food in 4 mice under each condition. ** p<0.005, ***p<0.0005.

FIG. 25 show that Gly-low reduced body weight gain and food consumption in 3×Tg-AD mouse. Three month old 3×Tg-AD female mice treated with Gly-low for 6 months were tested for body weight gain (top panel) and food intake (bottom panel) twice in a week. N=9 mice per group, p<0.0001. Similar results were seen in males.

FIG. 26, panels A-B shows that Gly-low improves memory in 3×Tg-AD mice. Panel A) Six months old 3×Tg-AD mice treated with Gly-low for 3 months were tested for memory using Y-maze test. Percentage of alterations indicates the memory function and number of entries to different Y-maze arms (panel B) indicates the activity. Observed results suggested that Gly-low treatment improved the memory as well as activity in 3×Tg-AD mice model

### DETAILED DESCRIPTION

In various embodiments, methods and compositions are provided herein for inducing or increasing weight loss or reducing or preventing weight gain in a mammal.

Without being bound to a particular theory, we believe that the MGO derived AGE, MG-H1 (hydroimidazolone-1), enhances food intake and promotes glucose consumption, potentially fueling both homeostatic and hedonic mechanisms to promote obesity. We manipulated the MGO-MG-H1 pathway up and down to test how this pathway influences homeostatic feeding and leptin responsiveness. Additionally, we tested the role of the MGO/MG-H1 axis in regulating hedonic preference for sugar and carbohydrates in general.

We used C. elegans to screen for compounds that mitigate the detrimental phenotypes in the worm glod-4 mutant. Using a natural product library (TIM TEC® NPL-640), 640 compounds were screened and 15 were identified that ameliorate the deleterious effects of MGO toxicity in both C. elegans and mammalian neuronal cells. This included alpha-lipoic acid (ALA), a drug already prescribed for diabetic neuropathy in certain countries (see, e.g., Giacco et al. (2014) Diabetes 63: 291-299). Next, we examined whether we could enhance ALA's ability to detoxify AGEs. We combined ALA with 14 other compounds, including some from our screen, to ameliorate the deleterious phenotypes of MGO toxicity in neurons. We meticulously tested over a hundred binary, ternary, quaternary, and quinary combinations of 14 compounds and validated them in multiple cell lines.

We demonstrated a that a cocktail of five GRAS (generally regarded as safe) compounds, including ALA that work incredibly well to ameliorate MGO-induced stress. These compounds are lipoic acid (designated “L” herein), nicotinamide (designated “N” herein), thiamine (designated “T” herein), piperine (designated “Pi” herein), and pyridoxamine (designated “Py” herein).

Even at the lowest (1 nM) concentration, which includes only 200 μM of each member of the cocktail, the mixture provides significant protection against neural damage. By generating a cocktail (combination of agents/drugs), we could reduce individual drug concentrations by five-fold while increasing their overall effectiveness. This cocktail (also referred to herein as GLYLO™ or GLY-LOW) also worked remarkably well in two different mammalian dopaminergic cell lines. Again, synergistic effects were noted, as, despite each member being present at only ⅕ the dose, they worked better in the cocktail than did any of them when given alone at 1× dose. It is also notable that single compounds like ALA and nicotinamide protect against obesity. Based on the data provided herein, we believe that the 5-compound anti-AGEs cocktail is much more effective and readily translates from neuroprotection in assays of neuronal damage, and assays designed to explore food intake to daily use in subjects.

We tested whether the cocktail of MGO-lowering anti-AGEs compounds would reduce food intake in db/db mice, that lack the leptin receptor and rapidly develop severe obesity. With fundamentally impaired hypothalamic melanocortin pathway activity, this model is useful because it also develops severe hyperglycemia with features of frank diabetes. We administered the anti-AGEs cocktail, using a 1×, 0.5×, or 0.25× dose, chronically to obese db/db mice with established diabetes, and then tracked them for six months. Remarkably, a drastic decrease in the body weights of the mice was observed soon after beginning anti-AGEs treatment. Indeed, the mice treated with the highest (1×) dose reached a plateau in body weight commensurate with that of an age-matched WT control mouse (not shown). This decrease was coupled to a dose-dependent decrease in cumulative food consumption over the same treatment period. Although we found associated dose-dependent effects in rescuing several other metabolic and secondary diabetic complications.

In view of these results, in certain embodiments, methods of inducing or increasing weight loss or reducing or preventing weight gain in a mammal are provided. In various embodiments, the methods comprise administering to a mammal an effective amount of at least two agents, or at least 3 agents, or at least 4 agents, or five agents selected from the group consisting of lipoic acid and/or a lipoic acid analog, nicotinamide and/or a nicotinamide analog, thiamine and/or a thiamine analog, piperine and/or a piperine analog, pyridoxamine and/or a pyridoxamine analog. In certain embodiments the agents are administered separately, while in other embodiments, the agents can be administered as a combined formulation. In certain embodiments, the combination of agents provides a synergistic effect in the induction or increase of weight loss or the reduction or prevention of weight gain.

It was additionally discovered that the combinations of agents described herein can reduce carbohydrate consumption by said mammal and in various embodiments this does not substantially alter lipid consumption by mammal. Thus, the combinations of agents described herein are able to reduce blood glucose in a mammal and to increase the amount of insulin release in a mammal with diabetes or pre-diabetes, or to restore amount of insulin release in a mammal with diabetes or pre-diabetes to substantially normal levels. It is also believed the combination of agents can decrease AIC in a mammal.

Accordingly, in various embodiments, method of ameliorating one or more symptoms of an obesity related disease in a mammal are provided where the method involves administering to the mammal an effective amount of at least two agents selected from the group consisting of lipoic acid and/or a lipoic acid analog, nicotinamide and/or a nicotinamide analog, thiamine and/or a thiamine analog, piperine and/or a piperine analog, pyridoxamine and/or a pyridoxamine analog. In certain embodiments, the obesity-related disease comprise one or more pathologies selected from the group consisting of nonalcoholic fatty liver disease (NAFLD), high blood pressure, high cholesterol, high blood sugar, heart disease, stroke, and obesity-related cancer. In certain embodiments, the obesity-related disease comprises NAFLD. In certain embodiments, the obesity-related disease comprises nonalcoholic steatohepatitis (NASH). In various embodiments, the combination of agents provides a synergistic effect in ameliorating one or more symptoms or, and/or slowing or stopping the progression of, and/or to curing said obesity-related disease. In various embodiments, the mammal is a mammal identified as having elevated triglycerides. In certain embodiments, the mammal is a mammal diagnosed as pre-diabetic. In certain embodiments, the mammal is a mammal diagnosed as having diabetes. In various embodiments, the effective amount of agents administered is an amount sufficient to ameliorate a complication of diabetes selected from the group consisting of diabetic neuropathy, cardiomyopathy, nephropathy, retinopathy, microvascular damage, and early mortality. In certain embodiments, the combination of agents provides a synergistic effect in ameliorating a complication of diabetes selected from the group consisting of diabetic neuropathy, cardiomyopathy, nephropathy, retinopathy, microvascular damage, and early mortality. In certain embodiments, the method produces a reduction in one or more advanced glycation end products (e.g., glyoxal/GO). In certain embodiments, the method produces a reduction in, or slows the accumulation of, methylglyoxal/MGO. In certain embodiments, the method produces a reduction in, or slows the accumulation of 3-deoxyglucosone/3DG.

In various embodiments, as illustrated by the examples provided herein, the combination of agents described herein find utility in improving survival and/or healthspan, improving voluntary activity, improving muscle strength, improving cognition and/or memory, reducing inflammation, upregulating neurotrophic factors (e.g., BDNF), and decreasing blood glucose and/or lower A1C. Accordingly, methods of use of the combinations of agents described herein for these various activities are provided.

As noted above, in certain embodiments, embodiments, the methods comprise administering to a mammal an effective amount of at least two agents, or at least 3 agents, or at least 4 agents, or five agents selected from the group consisting of lipoic acid and/or a lipoic acid analog, nicotinamide and/or a nicotinamide analog, thiamine and/or a thiamine analog, piperine and/or a piperine analog, pyridoxamine and/or a pyridoxamine analog. In certain embodiments the agents are administered separately, while in other embodiments, the agents can be administered as a combined formulation (e.g., GLYLO™).

It was also discovered that administration of hydroimidazolone N_{δ}-(5-hydro-5-methyl-4-imidazolon-2-yl) ornithine (MG-H1) to a mammal can increase food intake and induce weight gain. Accordingly, in certain embodiments, methods of inducing or increasing feeding and weight gain in a mammal are provided where the method comprises administering to the mammal an effective amount of hydroimidazolone N_{δ}-(5-hydro-5-methyl-4-imidazolon-2-yl) ornithine (MG-H1). In certain embodiments, the mammal is a mammal that has a pathology characterized by abnormal weight loss (e.g., dysphagia, painful mouth sores, newly applied orthodontic appliances, or loss of teeth, pyloric stenosis, hiatus hernia, coeliac disease, chronic pancreatitis, Crohn's disease, gastrointestinal infection, gastrointestinal fistulas, carcinoid disorders, intestinal hypermotility, hepatobiliary disease, food intolerance, medication induced weight loss, hyperthyroidism, Addison's disease, cancer (e.g., lymphoma, leukemia, carcinoma, sarcoma), heart failure, chronic respiratory disease, chronic kidney disease, liver failure, rheumatoid arthritis, systemic lupus erythematosus, acute infection, chronic infections (e.g., tuberculosis, HIV, parasitic infections, etc.), drug abuse, heavy smoking, stress-induced weight loss, depression, anorexia nervosa, food phobias, Parkinson's disease, etc.).

In certain embodiments, formulations (e.g., pharmaceutical formulations) for the practice of the methods described herein and kits for the practice of the methods described herein are provided.

### Active Agents.

As explained above, it was discovered that various combinations of the active agents described herein (e.g., lipoic acid and/or a lipoic acid analog, nicotinamide and/or a nicotinamide analog or nicotinamide metabolite (e.g., nicotinamide mononucleotide (NMN) or nicotinamide ribonucleotide (NMR), thiamine and/or a thiamine analog, piperine and/or a piperine analog, pyridoxamine and/or a pyridoxamine analog) are effective to ameliorate MGO toxicity in both C. elegans and mammalian animal models. Additionally, it was discovered that combinations of two or more, or three or more, or four or more, or five or more of these agents are effective to induce or increase weight loss and/or to reduce or to prevent weight gain in a mammal and the agents are synergistic in this activity. These agents are effective to prevent or ameliorate α-DC stress and these combinations of agents represent a viable option to address pathologies in diabetes and associated neurodegenerative conditions like Alzheimer's, Parkinson's disease, amyotrophic lateral sclerosis, Huntington's disease, prion diseases, and the like.

### Lipoic Acid and Lipoic Acid Analogs.

In certain embodiments the methods described herein can involve administration of lipoic acid and/or a lipoic acid analog in combination with one or more of, nicotinamide and/or a nicotinamide analog, and/or thiamine and/or a thiamine analog, and/or piperine and/or a piperine analog, and/or pyridoxamine and/or a pyridoxamine analog. Similarly, compositions described herein can comprise lipoic acid and/or a lipoic acid analog, and nicotinamide and/or a nicotinamide analog, and/or thiamine and/or a thiamine analog, and/or piperine and/or a piperine analog, and/or pyridoxamine and/or a pyridoxamine analog as described herein.

In certain embodiments the lipoic acid and/or lipoic acid analog comprises lipoic acid (1,2-dithiolane-3-pentanoic acid).

In certain embodiments the lipoic acid and/or lipoic acid analog comprises a lipoic acid analog. Lipoic acid analogs are well known to those of skill in the art and include, but are not limited to, bisnor-lipoic (1,2-dithiolane-3-propanoic), and tetranorlipoic (1,2-dithiolane-3-carboxylic) acids, a lipoic acid amide, and the like. Lipoic acid amides are described, inter alia, in U.S. Pat. No. 3,223,712, and U.S. Patent Pub. No: US 2007/0083054 A1). Illustrative lipoic acid amides include, but are not limited to lipoamide (6,8-dithiooctanoic amide), 2-(N,N-dimethylamine) ethylamido lipoate, and the like.

A number of lipoic acid analogs are also described in U.S. Pat. No. 6,090,842 which is incorporated herein by reference for the lipoic acid analogs described therein. Such analogs include, but are not limited to analogs according to Formula I:

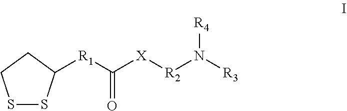

where R¹ and R² independently denote a methylene, ethylene or unbranched or branched C3-16 alkylene, alkenylene or alkynylene group which is unsubstituted or substituted with one or more halogen, hydroxyl or amine groups, where in the unbranched or branched C3-16 alkylene, alkenylene or alkynylene group an internal alkylene carbon atom in the carbon backbone thereof can be replaced by an oxygen atom; R³ and R⁴:
(i) independently denote (a) hydrogen, or (b) a methyl, ethyl, vinyl or unbranched or branched C3-16 alkyl, alkenyl or alkynyl group which is unsubstituted or substituted with one or more halogen, hydroxyl or amine groups, wherein in said unbranched or branched C3-16 alkyl, alkenyl or alkynyl group an internal alkylene carbon atom in the carbon backbone thereof can be replaced by an oxygen atom; or (c) a cycloalkyl, alkylcycloalkyl, alkenylcycloalkyl or alkynylcycloalkyl group having 5 to 16 carbon atoms which is unsubstituted or substituted with one or more halogen, hydroxyl or amine groups, or (d) an aryl, alkaryl, aralkyl, alkenylaryl, aralkenyl, alkynylaryl or aralkynyl group having 6 to 16 carbon atoms which is unsubstituted or substituted with one or more halogen, hydroxyl or amine groups, or
(ii) jointly with the nitrogen atom form a cyclic or aromatic amine which is unsubstituted or substituted with one or more alkyl, alkenyl, alkynyl, halogen, hydroxyl or amine groups;
X denotes O, S, —NH— or —NR⁵—, and R⁵ denotes methyl, ethyl, or unbranched or branched C3-16 alkyl, or a pharmaceutically acceptable salt thereof.

Other lipoic acid analogs described in U.S. Pat. No. 6,090,842 include compounds according to Formula II:

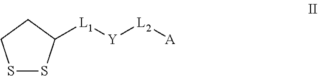

wherein L¹ and L² independently denote (i) a methylene group or a C₆₋₁₀ arylene group which is unsubstituted or substituted with a halogen, hydroxyl, amine or unbranched or branched C₃₋₁₆ alkyl, alkenyl or alkynyl group or (ii) a linking group having a carbon backbone that includes 2 to 16 carbon atoms, wherein a carbon atom in said carbon backbone can be replaced by an oxygen atom, an unsubstituted or substituted amine group, a sulfur atom, an unsubstituted or substituted C₆₋₁₀ aryl group or a combination thereof; Y denotes an ester, thioester, urethane or unsubstituted or alkyl-substituted amide linkage; and A denotes a group containing a nitrogen atom that is in equilibrium with a protonated form thereof; or a pharmaceutically acceptable salt thereof. Both the R and the S enantiomers are provided.

The foregoing lipoic acid analogs are illustrative and non-limiting. Using the teachings provided herein numerous other lipoic acid analogs will be available to one of skill in the art for use in the methods and compositions described herein.

### Nicotinamide and Nicotinamide Analogs.

In certain embodiments the methods described herein can involve administration of nicotinamide and/or a nicotinamide analog or nicotinamide metabolite (e.g., nicotinamide mononucleotide (NMN) or nicotinamide ribonucleotide (NMR), in combination with one or more of lipoic acid and/or a lipoic acid analog, thiamine and/or a thiamine analog, piperine and/or a piperine analog, and/or pyridoxamine and/or a pyridoxamine analog. Similarly, compositions described herein can comprise nicotinamide and/or a nicotinamide analog and lipoic acid and/or a lipoic acid analog, and/or thiamine and/or a thiamine analog, and/or piperine and/or a piperine analog, and/or pyridoxamine and/or a pyridoxamine analog as described herein.

In certain embodiments the nicotinamide and/or nicotinamide analog comprises nicotinamide. In certain embodiments, the nicotinamide component of the mixture comprises a nicotinamide metabolite (e.g., downstream metabolite). In certain embodiments, the downstream metabolite comprises nicotinamide mononucleotide (NMN). In certain embodiments, the downstream metabolite comprises nicotinamide ribonucleotide (NMR).

In certain embodiments the nicotinamide is provided as a nicotinamide analog. Nicotinamide analogs are well known to those of skill in the art. Illustrative nicotinamide analogs include, but are not limited to benzamide, 3-aminobenzamide, pyrazinamide, 3-acetamidobenzamide, 3-methoxybenzamide, 5-methyl-nicotinamide and the like.

In certain embodiments the nicotinamide analog comprises an O-benzyl nicotinamide analog as described in U.S. Patent Pub. No: 2011/0183980, which is incorporated herein by reference for the nicotinamide analogs described therein. In certain embodiments, such nicotinamide analogs include compounds according to Formula IV below:

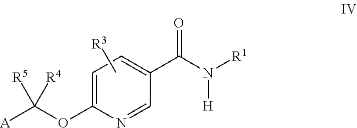

where R¹ is an C1 to C9 organic residue selected from alkyl, aryl, heteroaryl, cycloalkyl, heterocycloalkyl, cycloalkenyl, and heterocycloalkenyl, where R¹ is optionally substituted with one or more of halide, hydroxyl, trifluoromethyl, cyano, C1 to C4 alkoxy, thiol, C1 to C4 alkylsulfonyl, or C1 to C4 sulfonamide; R³ represents 0-1 substituents independently selected from C1 to C4 alkyl, C1 to C4 haloalkyl, halide, hydroxyl, trifluoromethyl, cyano, C1 to C4 alkoxy, thiol, C1 to C4 alkylsulfonyl, C1 to C4 carboxamide, and C1 to C4 sulfonamide; R⁴ and R⁵ are independently hydrogen or an C1 to C6 organic residue selected from alkyl, aryl, heteroaryl, cycloalkyl, heterocycloalkyl, cycloalkenyl, and heterocycloalkenyl, optionally substituted with one or more of halide, hydroxyl, trifluoromethyl, cyano, C1 to C4 alkoxy, thiol, C1 to C4 alkylsulfonyl, or C1 to C4 sulfonamide, or R⁴ and R⁵, together with the intermediate carbon, comprise an optionally substituted C3 to C6 cycloalkyl or heterocycloalkyl; and A is an optionally substituted C3 to C9 cyclic organic residue selected from aryl, heteroaryl, cycloalkyl, heterocycloalkyl, cycloalkenyl, and heterocycloalkenyl.

Illustrative compounds include, but are not limited to (R)-6-(benzyloxy)-N-(1-cyclohexylethyl)nicotinamide, (S)-6(benzyloxy)-N-(1-cyclohexylethyl)nicotinamide, (R)—N-(1-cyclohexylethyl)-6-(3-fluorobenzyloxy) nicotinamide, (S)—N-(1-cylohexylethyl)-6-(3-fluorobenzyloxy) nicotinamide, and the like.

In certain embodiments, niacin or a niacin analog can be provided instead of nicotinamide. In certain embodiments, nicotinic acid (pyridine-3-carboxylic acid), and other derivatives (e.g., inositol hexanicotinate), or precursors (e.g., nicotinamide riboside) can be utilized.

In certain embodiments a niacin analog can be used and may be viewed as also a nicotinamide analog comprises a nicotinamide analog. Niacin analogs are well known to those of skill in the art. For example, various niacin analogs are described, inter alia, in U.S. Pat. No. 8,377,971, which is incorporated herein by reference for the niacin analogs described herein. Illustrative niacin analogs described therein include analogs according to Formula III:

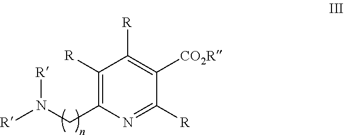

where R represents independently for each occurrence H, alkyl, alkenyl, alkynyl, aryl, heteroaryl, aralkyl, heteroaralkyl, fluoride, chloride, bromide, iodide, nitro, cyano, sulfonic acid, alkylsulfoxyl, arylsulfoxyl, heteroarylsulfoxyl, aralkylsulfoxyl, heteroaralkylsulfoxyl, alkenylsulfoxyl, alkynylsulfoxyl, alkylsulfonyl, arylsulfonyl, heteroarylsulfonyl, aralkylsulfonyl, heteroaralkylsulfonyl, alkenylsulfonyl, alkynylsulfonyl, hydroxyl, alkoxyl, aryloxyl, heteroaryloxyl, aralkyloxy, heteroaralkyloxy, alkenyloxy, alkynyloxy, thiol, alkylthio, arylthio, aralkylthio, heteroaralkylthio, alkenylthio, alkynylthio, formyl, acyl, formyloxy, acyloxy, formylthio, acylthio, amino, alkylamino, arylamino, heteroarylamino, aralkylamino, heteroaralkylamino, alkenylamino, alkynylamino, formylamino, acylamino, carboxylate, alkyloxycarbonyl, aryloxycarbonyl, heteroaryloxycarbonyl, aralkyloxycarbonyl, heteroaralkyloxycarbonyl, carboxamido, alkylaminocarbonyl, arylaminocarbonyl, heteroarylaminocarbonyl, aralkylaminocarbonyl, or heteroaralkylaminocarbonyl; R′ represents independently for each occurrence alkenyl, alkynyl, aryl, heteroaryl, aralkyl, heteroaralkyl, alkylsulfonyl, arylsulfonyl, heteroarylsulfonyl, aralkylsulfonyl, heteroaralkylsulfonyl, alkenylsulfonyl, alkynylsulfonyl, hydroxyl, alkoxyl, aryloxyl, heteroaryloxyl, aralkyloxy, heteroaralkyloxy, alkenyloxy, alkynyloxy, formyl, acyl, amino, alkylamino, arylamino, heteroarylamino, aralkylamino, heteroaralkylamino, alkenylamino, alkynylamino, formylamino, acylamino, alkyloxycarbonyl, aryloxycarbonyl, heteroaryloxycarbonyl, aralkyloxycarbonyl, heteroaralkyloxycarbonyl, alkylaminocarbonyl, arylaminocarbonyl, heteroarylaminocarbonyl, aralkylaminocarbonyl, or heteroaralkylaminocarbonyl; or the two instances of R′ taken together represent —(CH₂)₂—, —(CH₂)₃—, or —(CH₂)₆—; R″ represents independently for each occurrence H, alkyl, alkenyl, alkynyl, aryl, heteroaryl, aralkyl, or heteroaralkyl; and n is 1, 2, 3, or 4.

Particular niacin analogs described in U.S. Pat. No. 8,377,971 are shown in Table 1.

TABLE 1
Illustrative niacin analogs described in U.S. Pat. No. 8,377,971.

| Compound | Structure |
|---|---|
| 2037 | 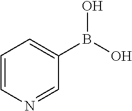 |
| 2040 | 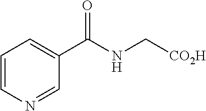 [SMILES: O=C(O)CNC(=O)c1cccnc1] |
| 2038 | 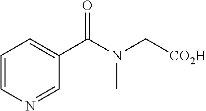 |
| 2230B | 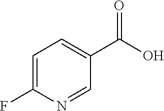 |
| 2035 | 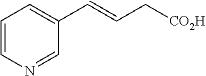 [SMILES: O=C(O)C/C=C/c1cccnc1] |
| 2230A | 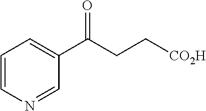 [SMILES: O=C(O)CCC(=O)c1cccnc1] |
| 2230C | 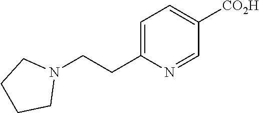 [SMILES: O=C(O)c1ccc(CCN2CCCC2)nc1] |
| 2035B | 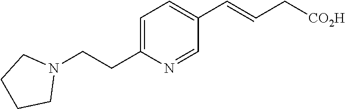 [SMILES: O=C(O)C/C=C/c1ccc(CCN2CCCC2)nc1] |
| S035C | 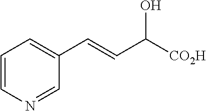 [SMILES: O=C(O)C(O)/C=C/c1cccnc1] |
| 2035D | 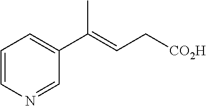 |
| 2805 | 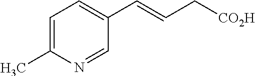 |
| 2805B | 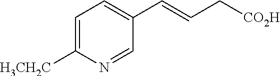 |
| 2805C | 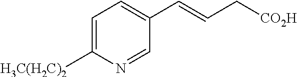 |
| 2805D | 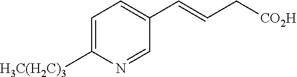 |

The foregoing nicotinamide analogs and/or niacin analogs are illustrative and non-limiting. Using the teachings provided herein numerous other nicotinamide analogs will be available to one of skill in the art for use in the methods and compositions described herein.

### Thiamine and Thiamine Analogs.

In certain embodiments the methods described herein can involve administration of thiamine and/or a thiamine acid analog in combination with one or more of, lipoic acid and/or a lipoic acid analog, and/or nicotinamide and/or a nicotinamide analog, and/or piperine and/or a piperine analog, and/or pyridoxamine and/or a pyridoxamine analog. Similarly, compositions described herein can comprise thiamine and/or a thiamine acid analog in combination with one or more of, lipoic acid and/or a lipoic acid analog, and/or nicotinamide and/or a nicotinamide analog, and/or piperine and/or a piperine analog, and/or pyridoxamine and/or a pyridoxamine analog.

In certain embodiments the thiamine or thiamine analog comprises thiamine.

In certain embodiments the thiamine or thiamine analog comprises a thiamine analog. Thiamine analogs are well known to those of skill in the art. Illustrative, but non-limiting thiamine analogs include, but are not limited to pyrithiamine, thiamine disulphide and acetylthiamine, oxythiamine, thiamine diphosphate, oxythiamine diphsophate, neopyrithiamine, 4-methyl-5-(hydroxymethyl)-thiazole (see, e.g., Buchman & Sargent (1945) J. Am/Chem. Soc., 67(3): 400-403, β-(4-Methylthiazolyl-5)-alanine (see, e.g., Buchman & Richardson (1939) J. Am. Chem. Soc. 61(4): 891-893), 3-(2, 4-dioxo-1, 2, 3, 4-tetrahydro-5-pyrimidinyl) methyl-4-methyl-5-(2-hydroxyethyl) thiazolium nitrate (see, e.g., Yamada & Achiwa (1961) Chem. Pharm. Bull., 9(2): 119-123), and the like.

The foregoing thiamine analogs are illustrative and non-limiting. Using the teachings provided herein numerous other thiamine analogs will be available to one of skill in the art for use in the methods and compositions described herein.

### Piperine and Piperine Analogs

In certain embodiments the methods described herein can involve administration of piperidine and/or a piperidine analog in combination with one or more of, lipoic acid and/or a lipoic acid analog, and/or nicotinamide and/or a nicotinamide analog, and/or thiamine and/or a thiamine analog, and/or pyridoxamine and/or a pyridoxamine analog. Similarly, compositions described herein can comprise piperidine and/or a piperidine analog in combination with one or more of, lipoic acid and/or a lipoic acid analog, and/or nicotinamide and/or a nicotinamide analog, and/or thiamine and/or a thiamine analog, and/or pyridoxamine and/or a pyridoxamine analog as described herein.

In certain embodiments the piperidine and/or a piperidine analog analog comprises piperidine.

In certain embodiments the piperidine and/or a piperidine analog analog comprises a piperidine analog. Piperine analogs are well known to those of skill in the art. Illustrative piperine analogs include, but are not limited to 1-(3,4-methylenedioxyphenyl)-penta-2E,4E-dienoic acid methyl ester (3a), 1-E,E-piperinoyl-isobutylamine, and 1-(3,4-methylenedioxyphenyl)-pentanoic acid cyclohexyl amide (see, e.g., Venkatasamy et al. (2004) Bioorg. Med. Chem., 12(8): 1905-1920). Additional piperine analogs are described, inter alia, in Bhardwaj & Dubey (2017) Org. Med. Chem., IJ 3(2): OMCIJ.MS.ID.555606) some of which are shown in Table 2, below.

Table 2. Illustrative, but non-limiting examples of piperine analogs.

Compound Structure

TABLE 2
Illustrative, but non-limiting examples of piperine analogs.

| Compound | Structure |
|---|---|
| verapamil | 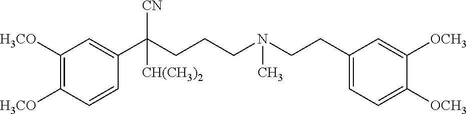 [SMILES: COc1ccc(CCN(C)CCCC(C#N)(c2ccc(OC)c(OC)c2)C(C)C)cc1OC] |
| sv10 | 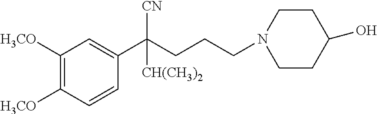 [SMILES: COc1ccc(C(C#N)(CCCN2CCC(O)CC2)C(C)C)cc1OC] |
| sv20 | 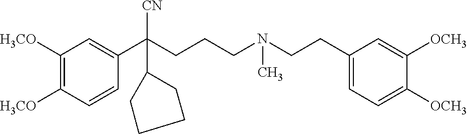 [SMILES: COc1ccc(CCN(C)CCCC(C#N)(c2ccc(OC)c(OC)c2)C2CCCC2)cc1OC] |
| sv21 | 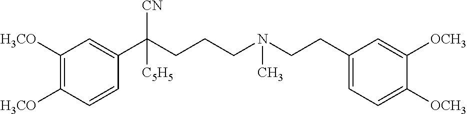 |
| sv9 | 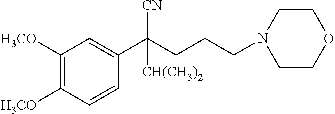 [SMILES: COc1ccc(C(C#N)(CCCN2CCOCC2)C(C)C)cc1OC] |
| sv12 | 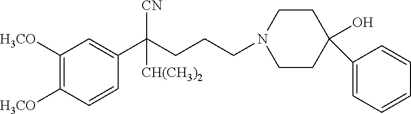 [SMILES: COc1ccc(C(C#N)(CCCN2CCC(O)(c3ccccc3)CC2)C(C)C)cc1OC] |
| sv5 | 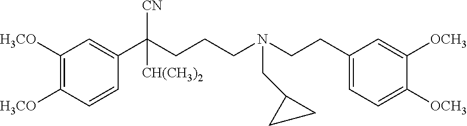 [SMILES: COc1ccc(CCN(CCCC(C#N)(c2ccc(OC)c(OC)c2)C(C)C)CC2CC2)cc1OC] |
| sv17 | 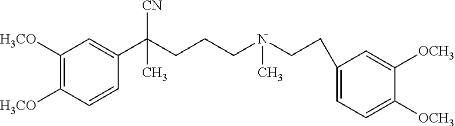 [SMILES: COc1ccc(CCN(C)CCCC(C)(C#N)c2ccc(OC)c(OC)c2)cc1OC] |
| sv7 | 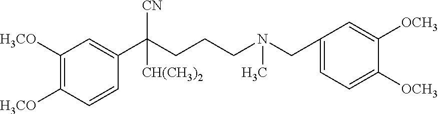 [SMILES: COc1ccc(CN(C)CCCC(C#N)(c2ccc(OC)c(OC)c2)C(C)C)cc1OC] |
| sv8 | 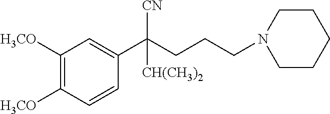 [SMILES: COc1ccc(C(C#N)(CCCN2CCCCC2)C(C)C)cc1OC] |
| sv4 | 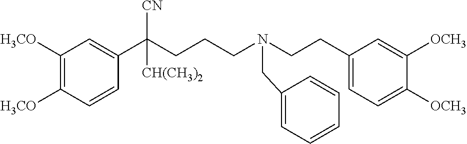 [SMILES: COc1ccc(CCN(CCCC(C#N)(c2ccc(OC)c(OC)c2)C(C)C)Cc2ccccc2)cc1OC] |
| sv3 | 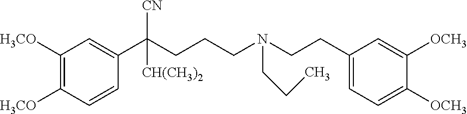 [SMILES: CCCN(CCCC(C#N)(c1ccc(OC)c(OC)c1)C(C)C)CCc1ccc(OC)c(OC)c1] |
| sv2 | 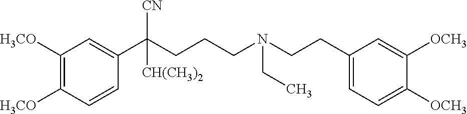 [SMILES: CCN(CCCC(C#N)(c1ccc(OC)c(OC)c1)C(C)C)CCc1ccc(OC)c(OC)c1] |
| sv15 | 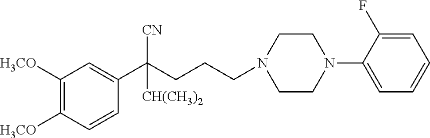 [SMILES: COc1ccc(C(C#N)(CCCN2CCN(c3ccccc3F)CC2)C(C)C)cc1OC] |
| sv19 | 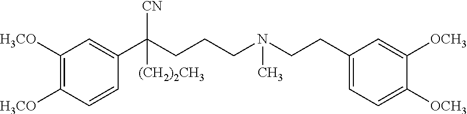 |
| sv18 | 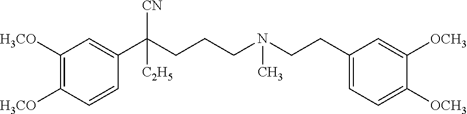 [SMILES: CCC(C#N)(CCCN(C)CCc1ccc(OC)c(OC)c1)c1ccc(OC)c(OC)c1] |
| sv16 | 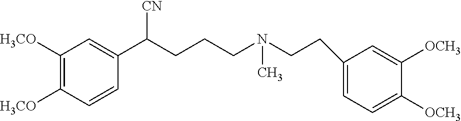 [SMILES: COc1ccc(CCN(C)CCCC(C#N)c2ccc(OC)c(OC)c2)cc1OC] |
| sv1 | 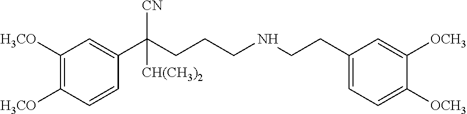 [SMILES: COc1ccc(CCNCCCC(C#N)(c2ccc(OC)c(OC)c2)C(C)C)cc1OC] |
| sv14 | 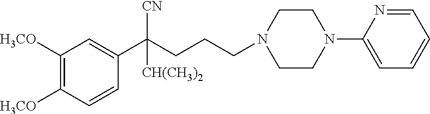 [SMILES: COc1ccc(C(C#N)(CCCN2CCN(c3ccccn3)CC2)C(C)C)cc1OC] |
| sv16 | 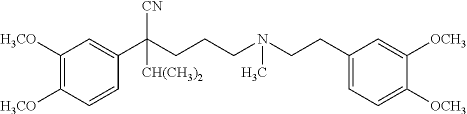 [SMILES: COc1ccc(CCN(C)CCCC(C#N)(c2ccc(OC)c(OC)c2)C(C)C)cc1OC] |
| sv13 | 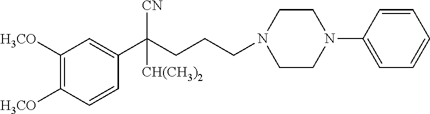 [SMILES: COc1ccc(C(C#N)(CCCN2CCN(c3ccccc3)CC2)C(C)C)cc1OC] |
| sv11 | 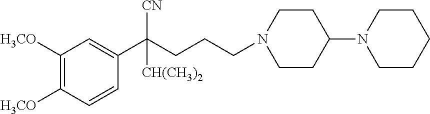 [SMILES: COc1ccc(C(C#N)(CCCN2CCC(N3CCCCC3)CC2)C(C)C)cc1OC] |

Additional piperine compounds described by Bhardwaj & Dubey (2017), supra., include P1057, P622, P545, P725, P557, P2, P4, P1045, P1090, P8, P594, P1087, P1088, P28, P12, P1118, P1, P1122, P677, P1120, P1121, P6, P1112, P1119, P1117, P1116, P1070, P1114, P569, P32, P5, P1084, P1069, P593, P10, P9, P665, P7, P17, P1123, P11, P1080, P33, P743, P16, P1078, P27, P604, P25, P29, 1073, P26, P30, P707, P752, P22, P670, P23, P21, P546, P3, P581, P15, P636, P20, P737, P649, and P689, and the use of any one or more of these compounds is contemplated as well.

The foregoing piperine analogs are illustrative and non-limiting. Using the teachings provided herein numerous other piperine analogs will be available to one of skill in the art for use in the methods and compositions described herein.

### Pyridoxamine and Pyridoxamine Analogs

In certain embodiments the methods described herein can involve administration of pyridoxamine and/or a pyridoxamine analog in combination with one or more of lipoic acid and/or a lipoic acid analog, and/or nicotinamide and/or a nicotinamide analog, and/or thiamine and/or a thiamine analog, and/or piperine and/or a piperine analog. Similarly, compositions described herein can comprise pyridoxamine and/or a pyridoxamine analog in combination with one or more of lipoic acid and/or a lipoic acid analog, and/or nicotinamide and/or a nicotinamide analog, and/or thiamine and/or a thiamine analog, and/or piperine and/or a piperine analog as described herein.

In certain embodiments the pyridoxamine and/or a pyridoxamine analog comprises pyridoxamine.

In certain embodiments the pyridoxamine and/or a pyridoxamine analog comprises a pyridoxamine analog. Illustrative pyridoxamine analogs include but are not limited to salicylamine (O-hydroxybenzylamine), thiosalicylamine (0-mercaptobenzylamine), and 3-hydroxy-4-aminomethylpyridine, 3-hydroxy-4-aminomethylpyridine, pyridoxamine phosphate, pyridoxal 5 phosphate, pyridoxine, 1-methylpyridoxamine chloride, pyridoxamine phosphate, 1-methyl-3-hydroxy-4-aminomethylpyridinium chloride, and the like. In certain embodiments the pyridoxamine analogs include but are not limited to alkyl-pyridoxamines (alkyl-PM), e.g., as described by Venkataraman et al. (2016) Chem Res Toxicol. 28(7): 1469-1475. Illustrative pyridoxamines are described by Venkataraman et al. and include, for example, alkylypridoxmines according to Formula V:

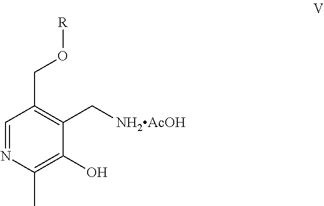

where R is a C5-C10 alkyl. In certain embodiments R is C₅H₁₁ (pentyl-PM), C₆H₁₃ (hexyl-PM), or C₇H₁₅ (heptyl-PM).

The foregoing pyridoxamine analogs are illustrative and non-limiting. Using the teachings provided herein numerous other pyridoxamine analogs will be available to one of skill in the art for use in the methods and compositions described herein.

### Pharmaceutical Formulations.

In certain embodiments combinations of two or more, or three or more, or four or more, or five or more compounds selected from the group consisting of lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog (or tautomer(s) or stereoisomer(s) thereof, or pharmaceutically acceptable salts, solvates, or clathrates thereof) are effective to prevent or ameliorate α-DC stress and these combinations of agents represent a viable option to address pathologies in diabetes and associated neurodegenerative conditions like Alzheimer's, and Parkinson's disease. In certain embodiments, the combination of compounds are used for ameliorating a pathology (e.g., ameliorating one or more symptoms of a pathology) characterized by elevated α-dicarbonyl compounds (e.g., Diabetes, Alzheimer's disease, Parkinson's disease, cataract formation, stroke, cardiovascular disease, etc.) or prophylactically slowing or stopping the onset of this pathology. In certain embodiments, the combinations of compounds are used for reducing the rate of formation and/or the levels of α-dicarbonyl compounds in a mammal. In certain embodiments, the combinations of compounds are used for reducing the amount of or slowing or stopping the formation and/or accumulation of, advanced glycation end products in a mammal.

The compounds (active agent(s)), combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog) described herein can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience, and as described above.

For example, a pharmaceutically acceptable salt can be prepared for any of the compounds (agent(s)) or combinations of agents described herein having a functionality capable of forming a salt. A pharmaceutically acceptable salt is any salt that retains the activity of the parent compound and does not impart any deleterious or untoward effect on the subject to which it is administered and in the context in which it is administered.

In various embodiments pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules.

Methods of formulating pharmaceutically active agents as salts, esters, amide, prodrugs, and the like are well known to those of skill in the art. For example, salts can be prepared from the free base using conventional methodology that typically involves reaction with a suitable acid. Generally, the base form(s) of the compound(s) of interest are dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. In certain embodiments addition salts of the active agents described herein include halide salts, such as may be prepared using hydrochloric or hydrobromic acids. Conversely, preparation of basic salts of the active agents (compounds) described herein are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic compounds, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active ingredient and in the acid or base is meant to make the proton transfer energetically favorable. When the pKa of the active ingredient and counterion are not significantly different, a solid complex may form but may rapidly disproportionate (i.e., break down into the individual entities of compound and counterion) in an aqueous environment.

In certain embodiments, the counterion is typically a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog), or tautomer(s) or stereoisomer(s) thereof, or pharmaceutically acceptable salts, solvates, or clathrates of said agents, are useful for parenteral administration, topical administration, oral administration, nasal administration (or otherwise inhaled), rectal administration, or local administration, such as by aerosol or transdermally, for prophylactic and/or therapeutic treatment of one or more of the pathologies/indications described herein (e.g., pathologies characterized by the accumulation of advanced glycation endproducts).

In various embodiments the each or a plurality of active agents described herein can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable materials, particularly of use in the preparation of tablets, capsules, gel caps, and the like include, but are not limited to binders, diluent/fillers, disintegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof), or tautomer(s) or stereoisomer(s) thereof, or pharmaceutically acceptable salts, solvates, or clathrates of said active agents, or derivatives, analogs, or prodrugs thereof) and the resulting composition is compressed. Where necessary the compressed product is coated, e.g., using known methods for masking the taste or for enteric dissolution or sustained release. Suitable coating materials include, but are not limited to ethyl-cellulose, hydroxymethylcellulose, POLYOX®yethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physiochemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In various embodiments, the pharmaceutical compositions (formulations) can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, mucoadherent films, topical varnishes, lipid complexes, etc.

Pharmaceutical compositions comprising the active agents described herein (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof), or tautomer(s) or stereoisomer(s) thereof, or pharmaceutically acceptable salts, solvates, or clathrates of said agents, or derivatives, analogs, or prodrugs thereof) can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

In certain embodiments, the active agents described herein are formulated for oral administration. For oral administration, suitable formulations can be readily formulated by combining the active agent(s) with pharmaceutically acceptable carriers suitable for oral delivery well known in the art. Such carriers enable the active agent(s) described herein to be formulated as tablets, pills, dragees, caplets, lozenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents, and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

In one illustrative, but non-limiting embodiment, a pharmaceutical formulation for oral administration comprises alpha-lipoic acid, nicotinamide, thiamine (e.g., provided as thiamine mononitrate), piperine, and pyridoxamine (e.g., provided as pyridoxine HCL) (vitamin B6). In certain embodiments the alpha lipoic acid is present in an amount ranging from about 100 mg up to about 200 mg per unit formulation, and/or the nicotinamide is present in an amount ranging from about 100 mg up to about 300 mg per unit formulation, and/or the thiamine mononitrate is present in an amount ranging from about 50 mg up to about 200 mg per unit formulation, and/or the pyridoxamine HCL is present in an amount ranging from about 25 mg up to about 100 mg per unit formulation, and/or the piperine is present in an amount ranging from about 5 mg up to about 25 mg per unit formulation. In certain embodiments the formulation comprises about 150 mg alpha lipoic acid, about 200 mg nicotinamide, about 100 mg thiamine mononitrate, about 15 mg piperine, and about 50 mg pyridoxine HCL per unit dose. In certain embodiments, particularly for oral administration the formulation additional comprises a binder (e.g., microcrystalline cellulose), and/or a metallic salt (e.g., magnesium stearate), and/or silicon dioxide. In certain embodiments the unit dosage formulation is provided as a capsule (e.g., a gelatin capsul, a degradable polymer capsule, etc.).

For administration by inhalation, the active agent(s) can be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator may be formulated containing a powder mix of the compound and a suitable powder base such as lactose or starch.

In various embodiments the active agent(s) can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45, amphiphilic materials such as Suppocire AP and polyglycolized glyceride, and the like). The base is selected and compounded for a desired melting/delivery profile.

For topical administration the active agent(s) described herein (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof), or tautomer(s) or stereoisomer(s) thereof, or pharmaceutically acceptable salts, solvates, or clathrates of one or more of the compounds can be formulated as solutions, gels, ointments, creams, suspensions, and the like as are well-known in the art.

In certain embodiments the active agents described herein are formulated for systemic administration (e.g., as an injectable) in accordance with standard methods well known to those of skill in the art. Systemic formulations include, but are not limited to, those designed for administration by injection, e.g., subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. Such penetrants are generally known in the art. Injectable formulations and inhalable formulations are generally provided as a sterile or substantially sterile formulation.

In addition to the formulations described previously, the active agent(s) may also be formulated as a depot preparation. Such long-acting formulations can be administered by implantation (for example subcutaneously or intramuscularly) or by intramuscular injection. Thus, for example, the active agent(s) may be formulated with suitable polymeric or hydrophobic materials (for example as an emulsion in an acceptable oil) or ion exchange resins, or as sparingly soluble derivatives, for example, as a sparingly soluble salt.

In certain embodiments the active agent(s) described herein can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Alternatively, other pharmaceutical delivery systems can be employed. For example, liposomes, emulsions, and microemulsions/nanoemulsions are well known examples of delivery vehicles that may be used to protect and deliver pharmaceutically active compounds. Certain organic solvents such as dimethylsulfoxide also can be employed, although usually at the cost of greater toxicity.

In certain embodiments the active agent(s) described herein (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof), or tautomer(s) or stereoisomer(s) thereof, or pharmaceutically acceptable salts, solvates, or clathrates of said agent(s) are formulated in a nanoemulsion. Nanoemulsions include, but are not limited to, oil in water (O/W) nanoemulsions, and water in oil (W/O) nanoemulsions. Nanoemulsions can be defined as emulsions with mean droplet diameters ranging from about 20 to about 1000 nm. Usually, the average droplet size is between about 20 nm or 50 nm and about 500 nm. The terms sub-micron emulsion (SME) and mini-emulsion are used as synonyms.

Illustrative oil in water (O/W) nanoemulsions include, but are not limited to: Surfactant micelles—micelles composed of small molecules surfactants or detergents (e.g., SDS/PBS/2-propanol); Polymer micelles—micelles composed of polymer, copolymer, or block copolymer surfactants (e.g., Pluronic L64/PBS/2-propanol); Blended micelles—micelles in which there is more than one surfactant component or in which one of the liquid phases (generally an alcohol or fatty acid compound) participates in the formation of the micelle (e.g., octanoic acid/PBS/EtOH); Integral micelles—blended micelles in which the active agent(s) serve as an auxiliary surfactant, forming an integral part of the micelle; and Pickering (solid phase) emulsions—emulsions in which the active agent(s) are associated with the exterior of a solid nanoparticle (e.g., polystyrene nanoparticles/PBS/no oil phase).

Illustrative water in oil (W/O) nanoemulsions include, but are not limited to: Surfactant micelles—micelles composed of small molecules surfactants or detergents (e.g., dioctyl sulfosuccinate/PBS/2-propanol, isopropylmyristate/PBS/2-propanol, etc.); Polymer micelles—micelles composed of polymer, copolymer, or block copolymer surfactants (e.g., PLURONIC® L121/PBS/2-propanol); Blended micelles—micelles in which there is more than one surfactant component or in which one of the liquid phases (generally an alcohol or fatty acid compound) participates in the formation of the micelle (e.g., capric/caprylic diglyceride/PBS/EtOH); Integral micelles—blended micelles in which the active agent(s) serve as an auxiliary surfactant, forming an integral part of the micelle (e.g., active agent/PBS/polypropylene glycol); and Pickering (solid phase) emulsions—emulsions in which the active agent(s) are associated with the exterior of a solid nanoparticle (e.g., chitosan nanoparticles/no aqueous phase/mineral oil).

As indicated above, in certain embodiments the nanoemulsions comprise one or more surfactants or detergents. In some embodiments the surfactant is a non-anionic detergent (e.g., a polysorbate surfactant, a polyoxyethylene ether, etc.). Surfactants that find use in the present invention include, but are not limited to, surfactants such as the TWEEN®, TRITON®, and TYLOXAPOL® families of compounds.

In certain embodiments the emulsions further comprise one or more cationic halogen containing compounds, including but not limited to, cetylpyridinium chloride. In still further embodiments, the compositions further comprise one or more compounds that increase the interaction (“interaction enhancers”) of the composition with microorganisms (e.g., chelating agents like ethylenediaminetetraacetic acid, or ethylenebis(oxyethylenenitrilo)tetraacetic acid in a buffer).

In some embodiments, the nanoemulsion further comprises an emulsifying agent to aid in the formation of the emulsion. Emulsifying agents include compounds that aggregate at the oil/water interface to form a kind of continuous membrane that prevents direct contact between two adjacent droplets. Certain embodiments of the present invention feature oil-in-water emulsion compositions that may readily be diluted with water to a desired concentration without impairing their anti-pathogenic properties.

In addition to discrete oil droplets dispersed in an aqueous phase, certain oil-in-water emulsions can also contain other lipid structures, such as small lipid vesicles (e.g., lipid spheres that often consist of several substantially concentric lipid bilayers separated from each other by layers of aqueous phase), micelles (e.g., amphiphilic molecules in small clusters of 50-200 molecules arranged so that the polar head groups face outward toward the aqueous phase and the apolar tails are sequestered inward away from the aqueous phase), or lamellar phases (lipid dispersions in which each particle consists of parallel amphiphilic bilayers separated by thin films of water).

These lipid structures are formed as a result of hydrophobic forces that drive apolar residues (e.g., long hydrocarbon chains) away from water. The above lipid preparations can generally be described as surfactant lipid preparations (SLPs). SLPs are minimally toxic to mucous membranes and are believed to be metabolized within the small intestine (see e.g., Hamouda et al. (1998) J. Infect. Disease 180: 1939).

In certain embodiments the emulsion comprises a discontinuous oil phase distributed in an aqueous phase, a first component comprising an alcohol and/or glycerol, and a second component comprising a surfactant or a halogen-containing compound. The aqueous phase can comprise any type of aqueous phase including, but not limited to, water (e.g., deionized water, distilled water, tap water) and solutions (e.g., phosphate buffered saline solution or other buffer systems). The oil phase can comprise any type of oil including, but not limited to, plant oils (e.g., soybean oil, avocado oil, flaxseed oil, coconut oil, cottonseed oil, squalene oil, olive oil, canola oil, corn oil, rapeseed oil, safflower oil, and sunflower oil), animal oils (e.g., fish oil), flavor oil, water insoluble vitamins, mineral oil, and motor oil. In certain embodiments, the oil phase comprises 30-90 vol % of the oil-in-water emulsion (e.g., constitutes 30-90% of the total volume of the final emulsion), more preferably 50-80%. The formulations need not be limited to particular surfactants, however in certain embodiments, the surfactant is a polysorbate surfactant (e.g., TWEEN 20®, TWEEN 40@, TWEEN 60@, and TWEEN 80®), a pheoxypolyethoxyethanol (e.g., TRITON® X-100, X-301, X-165, X-102, and X-200, and TYLOXAPOL®), or sodium dodecyl sulfate, and the like.

In certain embodiments a halogen-containing component is present. the nature of the halogen-containing compound, in some embodiments the halogen-containing compound comprises a chloride salt (e.g., NaCl, KCl, etc.), a cetylpyridinium halide, a cetyltrimethylammonium halide, a cetyldimethylethylammonium halide, a cetyldimethylbenzylammonium halide, a cetyltributylphosphonium halide, dodecyltrimethylammonium halides, tetradecyltrimethylammonium halides, cetylpyridinium chloride, cetyltrimethylammonium chloride, cetylbenzyldimethylammonium chloride, cetylpyridinium bromide, cetyltrimethylammonium bromide, cetyldimethylethylammonium bromide, cetyltributylphosphonium bromide, dodecyltrimethylammonium bromide, tetradecyltrimethylammonium bromide, and the like

In certain embodiments the emulsion comprises a quaternary ammonium compound. Quaternary ammonium compounds include, but are not limited to, N-alkyldimethyl benzyl ammonium saccharinate, 1,3,5-Triazine-1,3,5(2H,4H,6H)-triethanol; 1-Decanaminium, N-decyl-N,N-dimethyl-, chloride (or) Didecyl dimethyl ammonium chloride; 2-(2-(p-(Diisobuyl)cresosxy)ethoxy)ethyl dimethyl benzyl ammonium chloride; 2-(2-(p-(Diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl dimethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C₁₂-C₁₆); alkyl dimethyl benzyl ammonium chloride (C₁₂-C₁₈); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldimethyl(ethylbenzyl) ammonium chloride (C₁₂₋₁₈); Di-(C₈₋₁₀)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl) octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride (and) Quat RNIUM 14; N,N-Dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Nanoemulsion formulations and methods of making such are well known to those of skill in the art and described for example in U.S. Pat. Nos. 7,476,393, 7,468,402, 7,314,624, 6,998,426, 6,902,737, 6,689,371, 6,541,018, 6,464,990, 6,461,625, 6,419,946, 6,413,527, 6,375,960, 6,335,022, 6,274,150, 6,120,778, 6,039,936, 5,925,341, 5,753,241, 5,698,219, an d5,152,923 and in Fanun et al. (2009) Microemulsions: Properties and Applications (Surfactant Science), CRC Press, Boca Ratan Fl.

In certain embodiments, one or more active agents described herein (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof) can be provided as a “concentrate”, e.g., in a storage container (e.g., in a premeasured volume) ready for dilution, or in a soluble capsule ready for addition to a volume of water, alcohol, hydrogen peroxide, or other diluent.

### Administration

In certain embodiments one or more active agents described herein (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof), or tautomer(s) or stereoisomer(s) thereof, or pharmaceutically acceptable salts, solvates, or clathrates of said compounds, are administered to a mammal in need thereof, e.g., to a mammal at risk for or suffering from a pathology characterized by the formation and/or accumulation of advanced glycation endproducts (AGEs). In certain embodiments the active agent(s) are administered to prevent or delay the onset of a pre-diabetic dysfunction, and/or to ameliorate one or more symptoms of a pre-diabetic dysfunction, and/or to prevent or delay the progression of a pre-diabetic condition or to diabetes. In certain embodiments one or more active agent(s) are administered for the treatment of diabetes, e.g., to reduce the severity of the disease, and/or to ameliorate one or more symptoms of the disease, and/or to slow the progression of the disease.

In various embodiments the active agent(s) described herein (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof), or tautomer(s) or stereoisomer(s) thereof, or pharmaceutically acceptable salts, solvates, or clathrates of said active agents, or derivatives, analogs, or prodrugs thereof) can be administered by any of a number of routes. Thus, for example they can be administered orally, parenterally, (intravenously (IV), intramuscularly (IM), depo-IM, subcutaneously (SQ), and depo-SQ), sublingually, intranasally (inhalation), intrathecally, transdermally (e.g., via transdermal patch), topically, ionophoretically or rectally.

In various embodiments the active agent(s) are administered in an amount/dosage regimen sufficient to exert a prophylactically and/or therapeutically useful effect in the absence of undesirable side effects on the subject treated (or with the presence of acceptable levels and/or types of side effects). The specific amount/dosage regimen will vary depending on the weight, gender, age and health of the individual; the formulation, the biochemical nature, bioactivity, bioavailability and the side effects of the particular compound(s) in the combination of compounds.

In certain embodiments the therapeutically or prophylactically effective amount may be determined empirically by testing the agent(s) in known in vitro and in vivo model systems for the treated disorder. A therapeutically or prophylactically effective dose can be determined by first administering a low dose, and then incrementally increasing until a dose is reached that achieves the desired effect with minimal or no undesired side effects.

In certain embodiments the agents described herein are administered in an effective amount (dosage). In certain embodiments an effective amount is an amount effective for ameliorating a pathology (e.g., ameliorating one or more symptoms of a pathology) characterized by elevated α-dicarbonyl compounds (e.g., Diabetes, Alzheimer's disease, Parkinson's disease, cataract formation, stroke, cardiovascular disease, etc.) or prophylactically slowing or stopping the onset or progression of this pathology. In certain embodiments an effective amount is an amount effective for reducing the rate of formation and/or the levels of α-dicarbonyl compounds in a mammal. In certain embodiments an effective amount is an amount effective for reducing the amount of or slowing or stopping the formation and/or accumulation of, advanced glycation end products, in a mammal.

In various embodiments, the nicotinamide and/or nicotinamide analog, or nicotinamide metabolite, when present, is administered in an amount of at least 100 mg/dose, or at least 150 mg/dose, or at least 200 mg/dose. In certain embodiments, the nicotinamide and/or nicotinamide analog, or nicotinamide metabolite, when present, is administered in at a dosage of about 200 mg/dose.

In various embodiments, the lipoic acid and/or lipoic acid analog, when present is administered in an amount of at least 50 mg/dose, or at least 100 mg/dose, or at least 150 mg/dose. In certain embodiments, the lipoic acid and/or lipoic acid analog, when present is administered in an amount of about 150 mg/dose.

In various embodiments, the piperine or piperine analog, when present, is administered in an amount of at least 5 mg/dose, or at least 10 mg/dose, or at least 15 mg/dose. In certain embodiments, the piperine or piperine analog, when present, is administered in an amount of about 15 mg/dose.

In various embodiments, the pyridoxine or pyridoxine analog, when present is administered in an amount of at least about 10 mg/dose, or at least about 25 mg/dose, or at least about 50 mg/dose. In certain embodiments, the pyridoxine or pyridoxine analog, when present is administered in an amount of about 50 mg/dose.

In various embodiments, the thiamine and/or a thiamine analog, when present, is administered in an amount of at least 50 mg/dose, or at least 100 mg/dose. In certain embodiments, the thiamine and/or thiamine analog is administered in an amount of about 100 mg/dose.

In certain embodiments, the subject is administered nicotinamide at about 200 mg/dose, lipoic acid at about 150 mg/dose, piperine at about 15 mg/dose, pyridoxine at about 50 mg/dose, and thiamine at about 100 mg/dose. In certain embodiments, these agents are administered separately. In certain embodiments, the agents are administered in a formulation comprising at least 2, or at least 3, or at least 4, or all 5 agents.

In certain embodiments, the dosages described above, are administered once a day, or twice a day, or 3 times/day, or 4 times/day.

In various embodiments, the active agent(s) described herein can be administered sublingually. In some embodiments, when given sublingually, the compounds and/or analogs thereof can be given one to four times daily in the amounts described above for IM administration.

In various embodiments, the active agent(s) described herein can be administered intranasally. When given by this route, the appropriate dosage forms are a nasal spray or dry powder, as is known to those skilled in the art. In certain embodiments, the dosage of compound and/or analog thereof for intranasal administration is the amount described above for IM administration.

In various embodiments, the active agent(s) described herein can be administered intrathecally. When given by this route the appropriate dosage form can be a parenteral dosage form as is known to those skilled in the art. In certain embodiments, the dosage of compound and/or analog thereof for intrathecal administration is the amount described above for IM administration.

In certain embodiments, the active agent(s) described herein can be administered topically. When given by this route, the appropriate dosage form is a cream, ointment, or patch. When administered topically, the dosage is from about 1.0 mg/day to about 200 mg/day. Because the amount that can be delivered by a patch is limited, two or more patches may be used. The number and size of the patch is not important as long as a therapeutically effective amount of compound be delivered as is known to those skilled in the art. The compound can be administered rectally by suppository as is known to those skilled in the art. In certain embodiments, when administered by suppository, the therapeutically effective amount is from about 1.0 mg to about 500 mg.

In various embodiments, the active agent(s) described herein can be administered by implants as is known to those skilled in the art. When administering the compound by implant, the therapeutically effective amount is the amount described above for depot administration.

In various embodiments the dosage forms can be administered to the subject 1, 2, 3, or 4 times daily. In certain embodiments it is preferred that the compound be administered either three or fewer times, more preferably once or twice daily. In certain embodiments, it is preferred that the agent(s) be administered in oral dosage form.

It should be apparent to one skilled in the art that the exact dosage and frequency of administration will depend on the particular condition being treated, the severity of the condition being treated, the age, weight, general physical condition of the particular patient, and other medication the individual may be taking as is well known to administering physicians who are skilled in this art.

While the compositions and methods are described herein with respect to use in humans, they are also suitable for animal, e.g., veterinary use. Thus certain organisms (subjects) contemplated herein include, but are not limited to humans, non-human primates, canines, equines, felines, porcines, rodents, ungulates, lagomorphs, and the like.

The foregoing formulations and administration methods are intended to be illustrative and not limiting. It will be appreciated that, using the teaching provided herein, other suitable formulations and modes of administration can be readily devised.

### Kits.

In various embodiments the active agents described herein (e.g., combinations of compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof), or tautomer(s) or stereoisomer(s) thereof, or pharmaceutically acceptable salts, solvates, or clathrates of said active agent(s), or derivatives, analogs, or prodrugs thereof) can be provided in kits. In certain embodiments the kits comprise the active agent(s) described herein enclosed in multiple or single dose containers. In certain embodiments the kits can comprises component parts that can be assembled for use. For example, one or more active agent(s) in lyophilized form and a suitable diluent may be provided as separated components for combination prior to use. In certain embodiments various active agents are provided in combined formulations. Thus, in certain embodiments a combined formulation may include 2 different active agents, or 3 different active agents, or 4 different active agents, or 5 different active agents. In certain embodiments all of the active agents are provided in substantially equal amounts. In certain other embodiments piperidine is present in a greater amount the compounds.

In certain embodiments the kit may include a plurality of containers, each container holding one or more unit doses of the compounds or combinations of compounds. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration, e.g., as described herein. Thus, for example, in one illustrative, but non-limiting embodiments a blister package is provided containing each of the compounds (e.g., lipoic acid and/or a lipoic acid analog, and/or nicotinamide and/or a nicotinamide analog, and/or thiamine and/or a thiamine analog, and/or piperine and/or a piperine analog, and/or pyridoxamine and/or a pyridoxamine analog and/or combinations thereof) in a separate blister on a common backing. In certain embodiments the blister package can comprise a single daily dosage of the desired combination of compounds.

In certain embodiments the kits can further comprise instructional/informational materials. In certain embodiments the instructional materials teach the use of the compounds contained in the kit for inducing or increasing weight loss or reducing or preventing weight gain in a mammal. In certain embodiments, the instructional materials teach the use of the compounds contained in the kit for reducing the amount of, or slowing or stopping the formation and/or accumulation of, advanced glycation end products in a mammal.

In certain embodiments the informational material(s) indicate that the administering of the compounds included therein can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. In certain embodiments the informational material(s) may indicate that anaphylaxis can be fatal and may occur when any foreign substance is introduced into the body. In certain embodiments the informational material may indicate that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

While the instructional materials typically comprise written or printed materials they are not limited to such. Any medium capable of storing such instructions and communicating them to an end user is contemplated herein. Such media include but are not limited to electronic storage media (e.g., magnetic discs, tapes, cartridges, chips), optical media (e.g., CD ROM), and the like. Such media may include addresses to internet sites that provide such instructional materials.

In some embodiments, the kits can comprise one or more packaging materials such as, for example, a box, bottle, tube, vial, container, sprayer, insufflator, intravenous (I.V.) bag, envelope, and the like; and at least one unit dosage form of an agent comprising active agent(s) described herein and a packaging material. In some embodiments, the kits also include instructions for using the composition as prophylactic, therapeutic, or ameliorative treatment for the disease of concern.

In some embodiments, the articles of manufacture can comprise one or more packaging materials such as, for example, a box, bottle, tube, vial, container, sprayer, insufflator, intravenous (I.V.) bag, envelope, and the like; and a first composition comprising at least one unit dosage form of an active agent described herein.

### EXAMPLES

The following examples are offered to illustrate, but not to limit the claimed invention.

### Example 1

C. elegans as a Model to Study the Effects of MGO Accumulation.

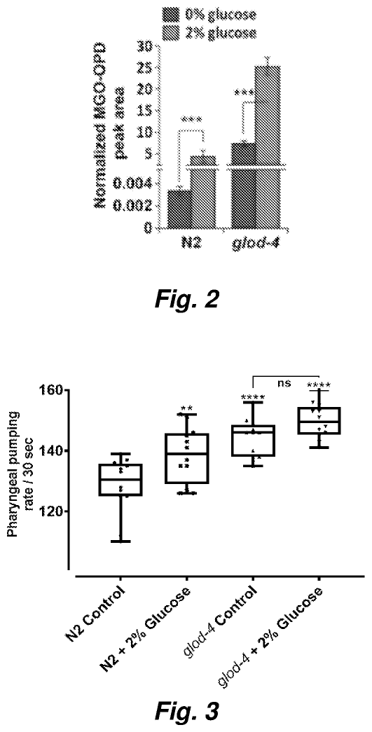

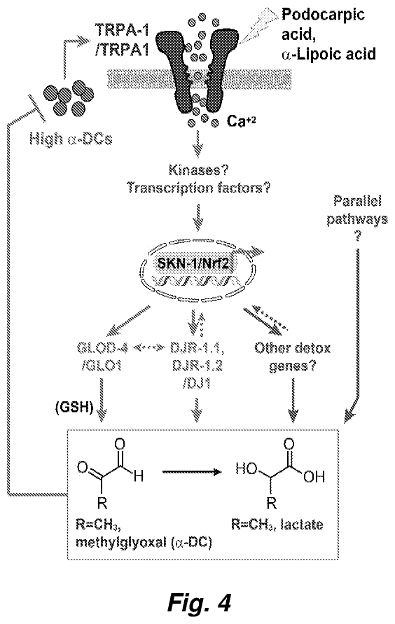

Given the significance of AGEs in diabetic pathologies, our lab established a C. elegans model that enables rapid assessment (<2 weeks) of the pathologies caused by the accumulation of MGO and downstream AGEs. We demonstrated using worms, which have contributed immensely to the genetics of how diet influences aging (Allaman et al. (2014) Front. Neurosci. 9: 23; Van Puyvelde et al. (2014) Nutr. Rev. 72: 638-650; Kapahi et al. (2016) Ageing Res. Rev. 39: 3-14), that mutants lacking glod-4 (ortholog of mammalian Glo1), which detoxifies MGO, reproduce several aspects of the pathological phenotype associated with AGE accumulation, including neuronal damage and reduced lifespan (Chaudhuri et al. (2016) Curr. Biol. 26: 3014-3025). Glo1 mutations in mice are associated with age-related diabetic cardiomyopathy and neuropathy (Mair & Dillin (2008) Annu. Rev. Biochem. 77: 727-754; Fontana & Partridge, (2015) Cell, 161: 106-118). Consistent with the role of glycolysis in producing MGO, we found that increased glucose in the media enhanced MGO levels in both the control (N2) and more so in glod-4 mutant worms (FIG. 2). Surprisingly, we found that glod-4 mutants also overeat (FIG. 3) and display increased bodily triglyceride accumulation (data not shown). This observation initiated our interest in developing the current project to examine the hypothesis that MGO derived AGEs drive feeding behavior in mice.

Identification of a Mixture of Compounds that Synergistically Lower MGO Toxicity.

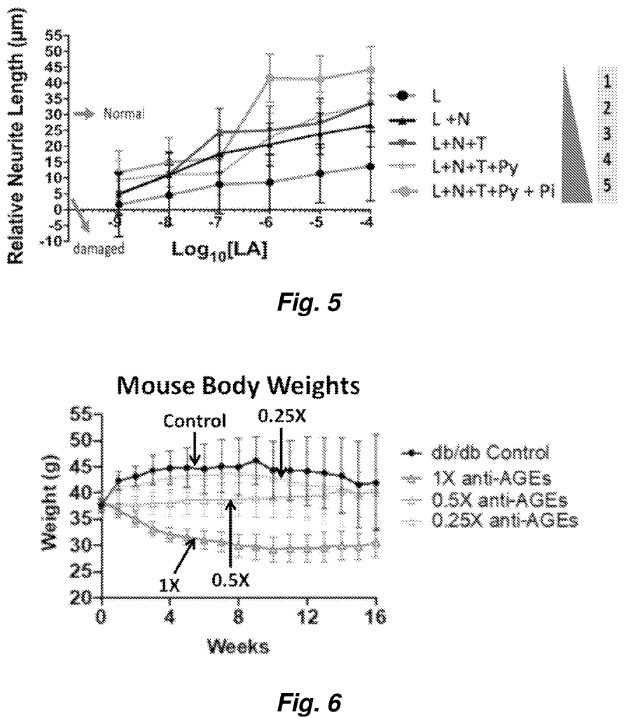

We used C. elegans to screen for compounds that mitigate the detrimental phenotypes in the worm glod-4 mutant. Using a natural product library (Tim Tec® NPL-640), the Kapahi laboratory screened 640 compounds and identified 15 compounds that ameliorate the deleterious effects of MGO toxicity in both C. elegans and mammalian neuronal cells¹. This included alpha-lipoic acid (ALA), a drug already prescribed for diabetic neuropathy in certain countries (see, e.g., Giacco et al. (2014) Diabetes 63: 291-299). Furthermore, a recent human trial with ALA showed its effectiveness in reducing weight (Bierhaus et al. (2012) Nat. Med. 18: 926-933), but its mechanism of action remains unclear. We determined that ALA activates the transient receptor potential cation channel (TRPA1), enhancing the expression of a family of evolutionarily conserved enzymes known as glyoxalases (DJ1 and GLO1), leading to MGO detoxification thus protecting worm mutants from the neurodegenerative and lifespan shortening effects of accumulation of AGEs (Chaudhuri et al. (2016) Curr. Biol. 26: 3014-3025). A model summarizing our findings from this study is shown in FIG. 4.

Next, we examined whether we could enhance ALA's ability to detoxify AGEs. We combined ALA with the other 14 hits from our screen to ameliorate the deleterious phenotypes of MGO toxicity in neurons, as we described (Id.). We meticulously tested over a hundred binary, ternary, quaternary, and quinary combinations of 14 compounds over 24 months and validated them in multiple cell lines.

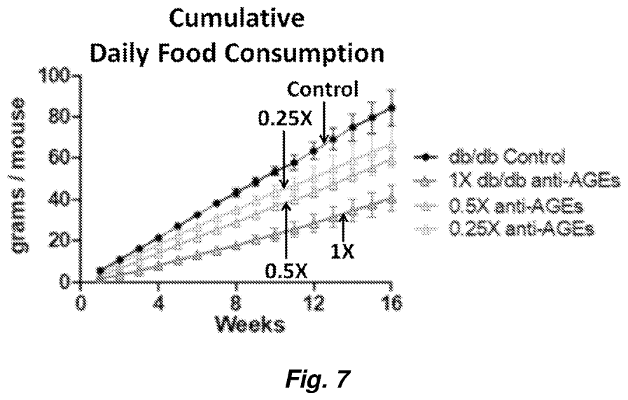

We demonstrated a that a cocktail of five GRAS (generally regarded as safe) compounds, including ALA that works incredibly well to ameliorate MGO-induced stress (FIG. 5). These compounds are lipoic acid (designated “L” herein), nicotinamide (designated “N” herein), thiamine (designated “T” herein), piperine (designated “Pi” herein), and pyridoxamine (designated “Py” herein).

Even at the lowest (1 nM) concentration, which includes only 200 μM of each member of the cocktail, the mixture results in significant protection against neural damage. By generating a cocktail, we could reduce individual drug concentrations by five-fold while increasing their overall effectiveness. This cocktail also worked remarkably well in two different mammalian dopaminergic cell lines. Again, synergistic effects were noted, as, despite each member being present at only ⅕ the dose, they worked better in the cocktail than did any of them when given alone at 1× dose. It is also notable that single compounds like ALA and nicotinamide protect against obesity (see, e.g., Bierhaus et al. (2012) Nat. Med. 18: 926-933; Gomes & Negrato (2014) Diabetology & Metabol. Synd. 6: 80; Bobe et al. (2020) J. Nutr. 150: 2336-2345; Malinska & Winiarska (2005) Postepy Hig Med Dosw (Online) 59: 535-543; Mitchell et al. (2018) Cell Metab. 27: 667-676). Based on our preliminary data, we expect that the 5-compound anti-AGEs cocktail will be much more effective and readily translate from protection in assays of neuronal damage, assays designed to explore food intake.

### MGO Lowering Anti-AGEs Compounds Rescue the Feeding Behavior and Metabolic Phenotypes in Leptin Receptor-Deficient Mice.

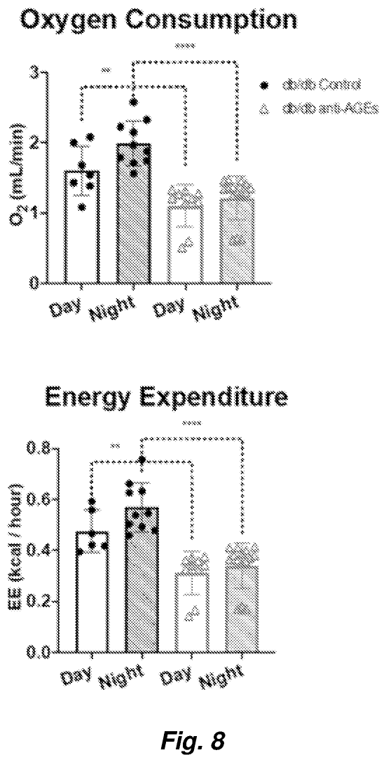

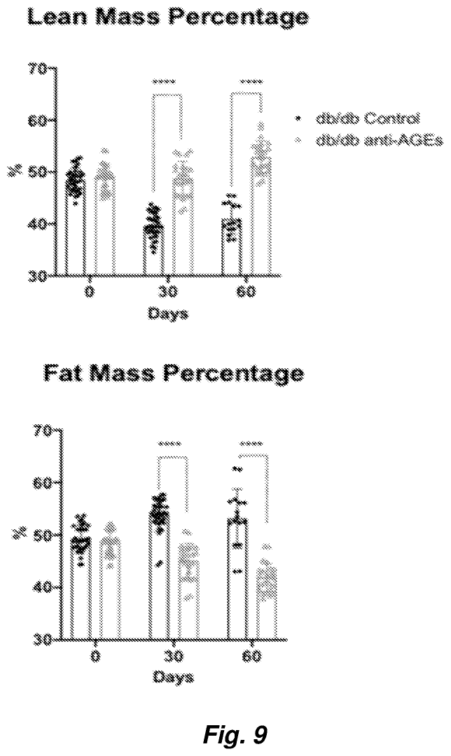

A small clinical trial with 2 compounds to activate GLO1 to lower MGO was useful in lowering weight gain in people (see, e.g., Canto et al. (2012) Cell Metab. 15: 838-847). We built on this by testing whether our cocktail of MGO-lowering anti-AGEs compounds would reduce food intake in db/db mice, which lack the leptin receptor and rapidly develop severe obesity (Trammell et al. (2016) Sci. Rep. 6: 26933). With fundamentally impaired hypothalamic melanocortin pathway activity, this model is useful because it also develops severe hyperglycemia with features of frank diabetes (see, e.g., Trammell et al. (2016) Sci. Rep. 6: 26933; Rabbani & Thornalley (2019) Antioxid. Redox Signal, 30: 354-374′ Lutz & Woods (2012) Curr. Protoc. Pharmacol. Chapter 5, Unit 561; Schwartz et al. (2000) Nature, 404: 661-671; Munzberg & Myers (2005) Nat. Neurosci. 8: 566-570). We administered our anti-AGEs cocktail, using a 1×, 0.5×, or 0.25× dose, chronically to obese db/db mice with established diabetes, and then tracked them for six months. Remarkably, we observed a drastic decrease in the body weights of the mice soon after beginning anti-AGEs treatment (FIG. 6). Indeed, the mice treated with the highest (1×) dose reached a plateau in body weight commensurate with that of an age-matched WT control mouse (not shown). This decrease was coupled to a dose-dependent decrease in cumulative food consumption over the same treatment period (FIG. 7). Although we found associated dose-dependent effects in rescuing several other metabolic and secondary diabetic complications, we focus on a subset of these results below for clarity sake.

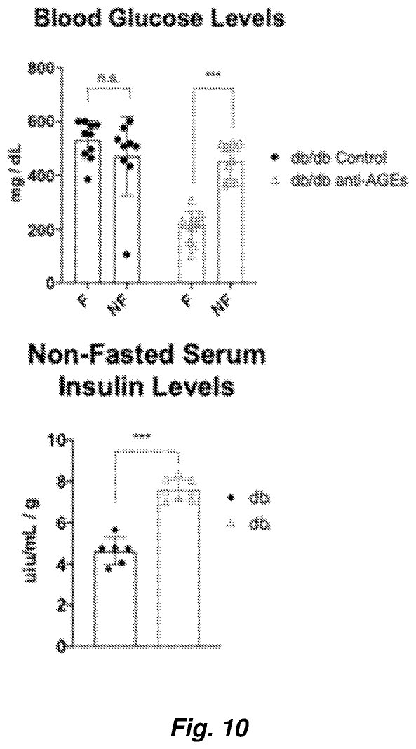

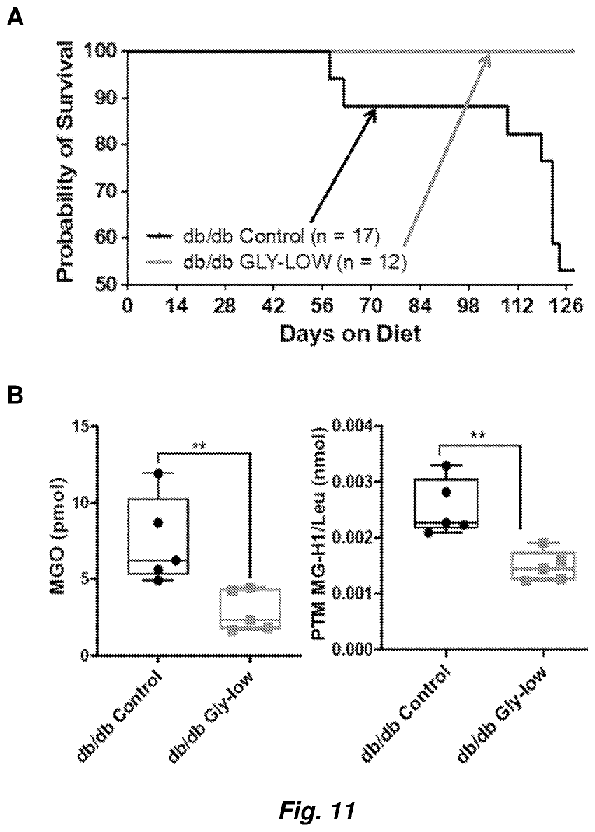

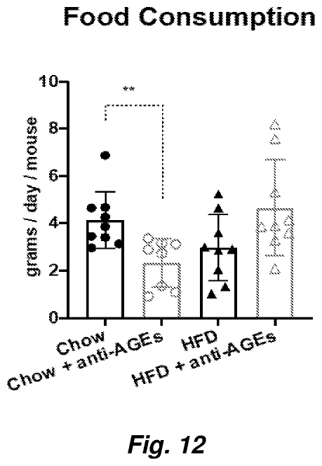

We measured the oxygen consumption, respiratory quotient (RQ), and physical activity of anti-AGEs treated and control db/db mice. We saw a significant reduction in oxygen consumption and total energy expenditure (FIG. 8), but no significant change in physical activity in the anti-AGEs treated group (data not shown). These results are consistent with the established concept that calorie restriction, while increasing healthspan, is associated with a compensatory lowering of oxygen consumption (see, e.g., Coleman & Herrmann (1999) Diabetologia, 42: 639-646). Next, we noted that lean muscle mass ion anti-AGWEs-treated db/db mice remained consistent (and even increased following 2 month treatment), while fat mass consistently decreased, indicating that the weight loss induced by anti-AGEs treatment was due to a specific decrease in fat mass (FIG. 9). Consistent with the observed weight loss anti-AGEs treated db/db mice had significantly reduced fasting blood glucose levels (FIG. 10). Moreover, non-fasting serum insulin levels were checked 20 weeks into the experiment (28 weeks of age) and were increased (p<0.0001) in db/db mice treated with the anti-AGEs compound vs. untreated controls (FIG. 10). Thus, lowering MGO-derived AGEs may also preserve insulin secretory capacity db/db mice.

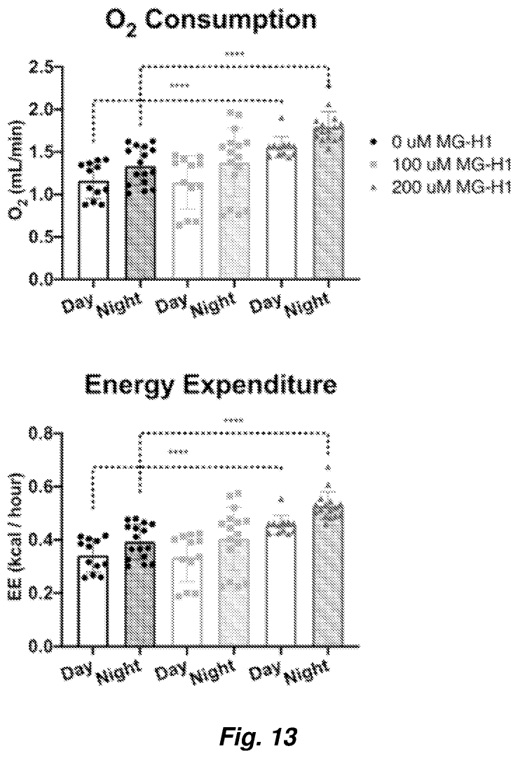

Two months into the study, at 16 weeks of age, untreated db/db mice were severely obese, and by almost six months, nearly half the db/db mice had died. However, not a single age-matched db/db mouse treated with the anti-AGEs cocktail died during this period (FIG. 11). Next, we asked if MG-H1 levels were increased in db/db mice. Our preliminary data show high levels of protein-bound MG-H1 in the livers and brains of db/db mice that were significantly reduced by treatment with the anti-AGEs cocktail (Gly-low combination) (data not shown). We analyzed the sera samples of control, and Gly-low treated and found a significant reduction in MGO and MG-H1 levels in Gly-Low treated db/db group compared to its controls (FIG. 11, panel B).

### Exogenous MG-H1 Enhances Food Intake and Weight Gain in C57/BL6 Mice and is Present in Tanycytes.

MGO-derived AGEs have been found in proteins with aging and in age-related diseases (see, e.g., Li et al. (2012) J. Neurol. Sci. 317: 1-5; Cone (2005) Nat. Neurosci. 8: 571-578; Mitchell et al. (2017) Oncotarget, 8: 17453-17474; Li et al. (2012) Neurobiol. Aging 33: 1400-1410). The Vlassara group showed that consuming MGO modified BSA (bovine serum albumin), which presumably elevates many AGEs, has significant negative health consequences, including insulin resistance, hyperglycemia, and reduced lifespan (see, e.g., Cai et al. (2012) Proc. Natl. Acad. Sci. USA, 109, 15888-15893; Batkulwar et al. (2018) ACS Chem. Neurosci. 9: 988-1000), in mice. However, MGO-treated BSA results in a mixture of AGEs, and the identity of the specific AGEs that mediate deleterious consequences remains unknown. To investigate the impact of specific AGEs, we provided synthesized individual AGEs and examined their biological effects. We adapted published procedures to synthesize four AGEs on a gram scale: carboxymethyl-lysine (CML), carboxyethyl-lysine (CEL), fructose-lysine (F-Ly), and methylglyoxal-derived hydroimidazolone MG-H1. CEL and MG-H1 are MGO derived AGEs while CML is glyoxal derived, and F-LY is glucose derived. Our preliminary data support the idea that feeding MG-H1, but not other AGEs, enhances food intake and increases triglyceride content in C. elegans (data not shown).

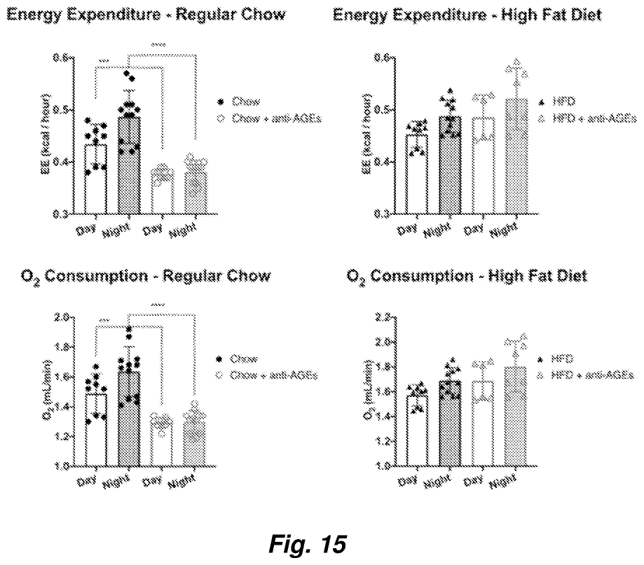

Next, we tested if MG-H1, which is detectable in human CSF and serum (Monnier et al. (2005) Ann. N. Y. Acad. Sci. 1043: 567-581), influences feeding in mice. We gave 12 week-old WT mice 100 μM or 200 μM MG-H1 via supplementation of drinking water for two weeks. The mice demonstrated a significant increase food intake and body weights at the higher dose (FIG. 13). These mice also demonstrated an increase in hourly food intake while maintaining relatively similar water intake (data not shown), ensuring similar rates of AGE consumption by all mice. Furthermore, the higher dose of MG-H1 induced a reactive increase in oxygen consumption and energy expenditure (FIG. 13). Our findings support our hypothesis that the MGO-derived MG-H1 enhances food intake to promote obesity.

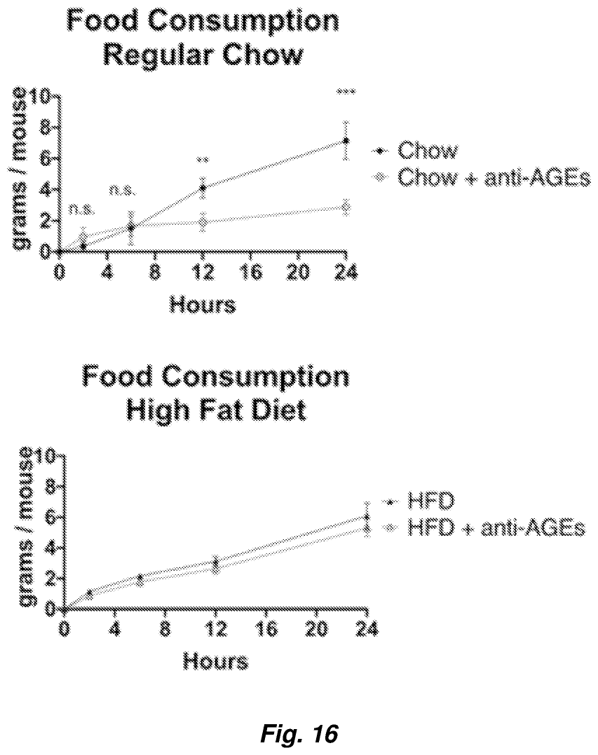

Given its significant effects on food intake, we asked if MG-H1 is detectable in the hypothalamus. The Koliwad Lab (Co-PI) used an MG-H1 specific antibody (Cell Biolabs) verified by the Vlassara lab65 to stain hypothalamic sections and saw highly specific staining for MG-H1 co-localizing with vimentin, a specific marker of tanycytes (FIG. 14). Tanycytes, are specialized glial cells that sense glucose and regulate the transport of peripheral factors, including ghrelin and leptin, into the cerebrospinal fluid (see, e.g., Al-Abed et al. (1999) Proc. Natl. Acad. Sci. USA, 96: 2385-2390; Cai et al. (2002) Mol. Med. 8: 337-346).

### Anti-AGEs Compounds Reduce Food Intake in High Carbohydrate but not a High-Fat Diet.

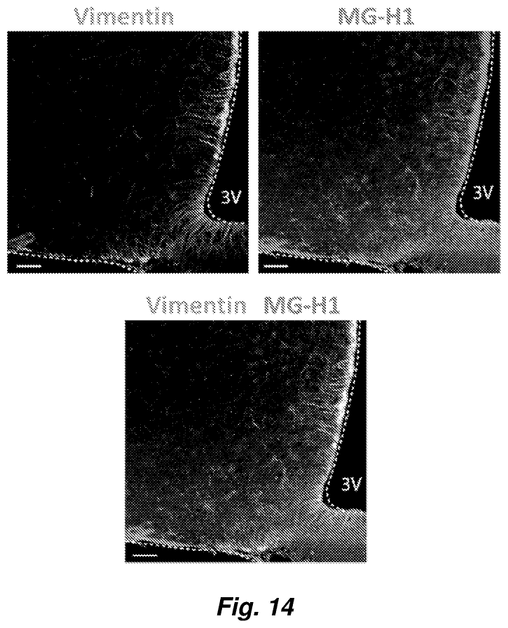

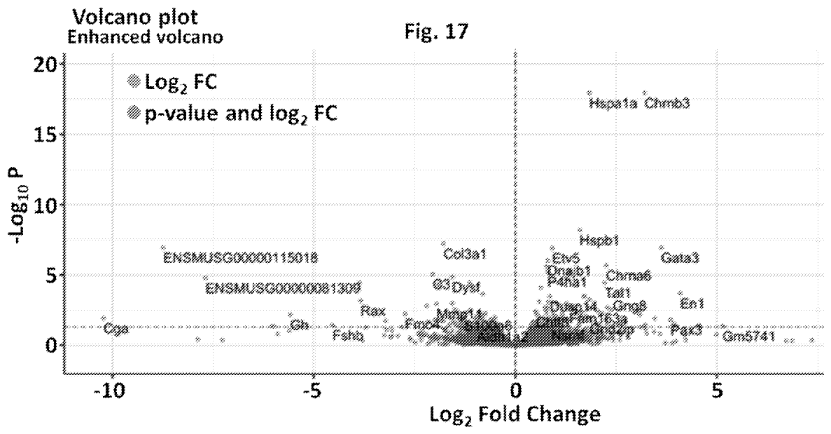

Next, we asked if the anti-AGEs compounds were capable of reduce food intake even in chow-fed, healthy C57BL6 mice. FIG. 12 shows a acute reduction in food intake among chow-fed mice over 5 days of treatment with anti-AGEs cocktail in metabolic cages. To our surprise, this acute effect did not manifest when tested in C57BL/6 mice fed a high fat diet, which is well established to induce obesity55 (FIG. 12). We obtained consistent findings when looking at oxygen consumption and energy expenditure. Anti-AGEs induced a reactive reduction in these parameters when mice were fed a normal chow diet, but not when they were fed a high-fat diet (FIG. 15). In pondering this acute dietary impact on the effects of anti-AGEs, we noted that the normal chow diet we used (Envigo, AIN-93G) included 64% of its calories from carbohydrates and only 17% from fat, whereas the high-fat diet (TD.06414) included 60% of calories from fat and only 21% from carbohydrates. Given that MGO is a byproduct of glycolysis, we hypothesize that lowering MGO and downstream AGEs would preferentially reduce carbohydrate consumption.

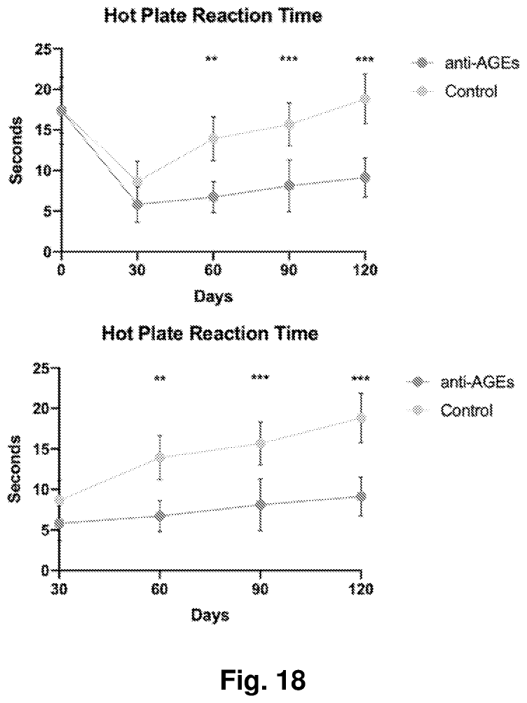

We wanted to confirm that our anti-AGEs cocktail did not impact food intake by inducing conditioned taste aversion. We observed that in mice allowed to refeed after a prolonged fast, the presence or absence of anti-AGEs did not reduce food consumption over the first six hours, although there was an expected reduction in food intake later on (FIG. 16, top). Moreover the anti-AGEs did not alter food intake when a high-fat diet was used for refeeding, which would not have been expected if the effects were due to taste aversion (FIG. 16, bottom). Most importantly, injecting our anti-AGEs cocktail intraperitoneally (i.p.) was also sufficient in lowering food intake (data not shown), which would also not be expected if this effect was due to taste aversion.

Mechanisms b which MG-H1 Enhances Food Intake.

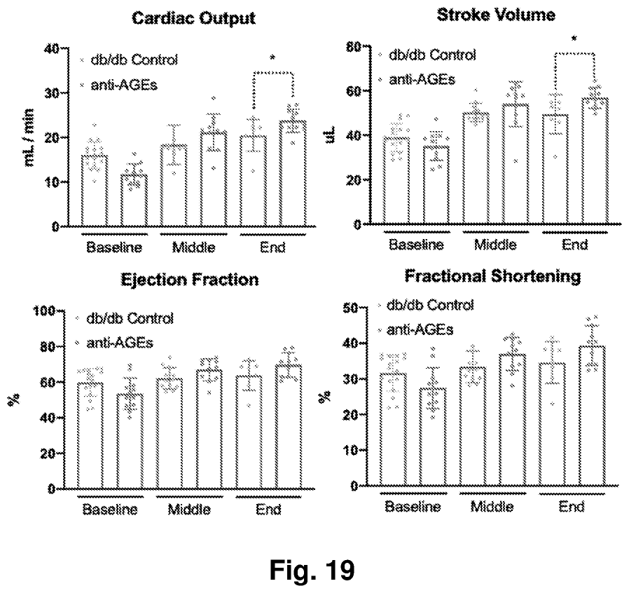

Our preliminary data show that dietary administration of synthetic AGEs can cause significant changes in gene expression in the hypothalamus (FIG. 17). We used Salmon quasi-mapper (Patro et al. (2017) Nat. Meth. 14: 417-419) to quantify bulk RNA sequencing of hypothalami from 3 control samples from db/db mice and 3 samples of db/db mice treated with anti-AGEs compounds. DEseq2 (Love et al. (2014) Genome Biol. 15: 550) was used to derive significantly differentially expressed genes (DEG) and associated their fold changes between the two groups. 239 significantly DEGs were found. Using the significant DEG, we applied gene ontology (GO) over-representation analysis (Yu et al. (2012) OMICS 16: 284-287) with the. Over-representation analysis uses a hypergeometric test to select gene ontology or pathways that are significantly up/down-regulated. We observed an over-representation of genes involved in neurotransmitter release. Analysis of individual groups of genes identified a striking increase in acetylcholine and dopamine signaling, chaperones, and a decrease in hormonal signaling (GH, FSH, and TSH). Interestingly, there was a significant reduction (11 fold) in rax, a critical marker for tanycyte, and its differentiation (Miranda-Angulo et al. (2014) J. Comp. Neurol. 522: 876-899). This change in rax fits nicely with our observation that increased MG-H1 is localized to tanycytes, while upon reduction of AGEs, a key tanycyte differentiation factor is reduced.

### Conclusions and Significance.

Homeostatic feeding drive is coordinated by neurons of the arcuate nucleus of the hypothalamus (ARH) (see, e.g., Rabbani & Thornalley (2019) Antioxid. Redox Signal, 30: 354-374; Prevot et al. (2018) Endocr. Rev. 39: 333-368). For example, under fasting conditions, decreased systemic levels of nutrients (e.g., glucose) and changes in related hormone levels (e.g., decreased leptin and increased ghrelin) serve to activate orexigenic neurons NPY/AgRP neurons and inhibit the anorexigenic neurons POMC neurons in the hypothalamus. The brain is traditionally considered an immunologically privileged site. However, the median eminence of the hypothalamus lacks a formal blood-brain barrier and is lined by a fenestrated vasculature, permitting sensitivity to communicating factors from the circulation. Tanycytes, a specialized glial cell type lining the wall of the third ventricle in the median eminence of the hypothalamus, acts as a gateway, dynamically regulating the transport of hormones like leptin and ghrelin to the CSF and secretion of neuropeptides into the hypophyseal portal vasculature by hypothalamic neurons, thus regulating blood-brain and blood-CSF exchanges (see, e.g., Al-Abed et al. (1999) Proc. Natl. Acad. Sci. USA, 96: 2385-2390; Cai et al. (2002) Mol. Med. 8: 337-346). Also, tanycytes possess the ability to sense blood glucose levels, and play a fundamental and active role in shuttling circulating metabolic signals to hypothalamic neurons that control food intake (Elizondo-Vega et al. (2015) J. Cell Mol. Med. 19: 1471-1482). However, the mechanisms by which changes in peripheral metabolism influence the neuronal circuits that modulate feeding behavior are poorly understood. Our preliminary data showing MG-H1 staining in tanycytes (FIG. 17) suggests its potential role in sensing glucose as its precursor MGO, is a byproduct of glycolysis.

Several studies have pointed to the increase in AGEs in diabetes (see, e.g., Jahan & Choudhary (2014) 25: 1267-1284; Vlassara & Uribarri (2014) Curr. Diabetes Rep. 14: 453; Garcia-Caceres et al. (2019) Nat. Neurosci. 22: 7-14; Patro et al. (2017) Nat. Meth. 14: 417-419), but their role in modulating food intake has not been explored. Our preliminary data suggest that compounds that lower MGO and associated AGEs can not only lower food intake but specifically lower carbohydrate intake, providing a potential mechanism for sugar addiction. These MGO lowering anti-AGEs compounds are an excellent tool to generate evidence in preclinical models that MGO lowering compounds in principle can reduce carbohydrate intake.

Obesity has serious health consequences by enhancing the risk of several diseases, including diabetes, cardiovascular diseases, and certain types of cancer. Furthermore, over 20% of total medical costs are associated with obesity, its impact on work productivity. Given that reducing carbohydrates (30-40%) in the diet is well established to extend lifespan and improve healthspan in mice, we expect that identifying and targeting pathways that may reduce the desire to overconsume carbohydrates would lead to meaningful health improvements. Our studies can impact clinical practice by providing MGO-lowering compounds as a potential treatment for weight gain and obesity. Moreover, given that our proposed cocktail contains agents classified as GRAS (Generally Regarded As Safe) by the FDA, including those already used as supplements, it should be relatively easy to translate our findings into human trials. Furthermore, our proposal could highlight MG-H1 as a potential biomarker for obesity and leptin resistance in humans, critical for future studies. Identifying MG-H1 binding proteins will could also yield unexpected novel targets for feeding mechanisms and obesity.

### Example 2

### Anti-AGEs Compound Ameliorates Diabetic Secondary Complications

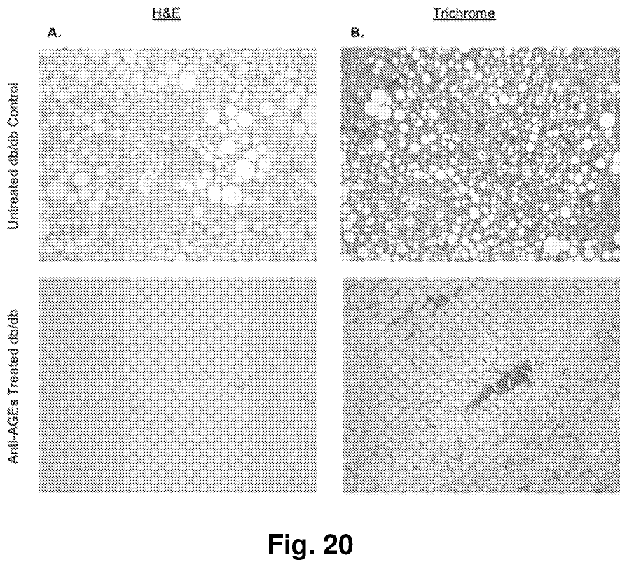

We wondered whether anti-AGEs treatment would improve diabetic secondary complications, such as peripheral neuropathy and cardiomyopathy, similar to its effect on metabolic phenotypes. To test for peripheral neuropathy, db/db mice were tested by an analgesia hot plate. This hot plate was set to 52° C. and animals were subjected to the heat until they gave a reaction to the stimulus. It was shown that by 60 days into treatment (16 weeks of age) treated db/db mice had significantly faster response times when challenged with an analgesia meter hot plate (FIG. 18), indicating lessening neuropathy in their limbs. While untreated db/db mice had progressively longer response times over the duration of the study, treated db/db mice stayed relatively similar. Results are shown both with and without baseline reading at time 0 days, as the mice appear to have undergone a sensitization between their first and second exposure to the hotplate (indicated by the faster response of animals at 30 days than at 0 days).

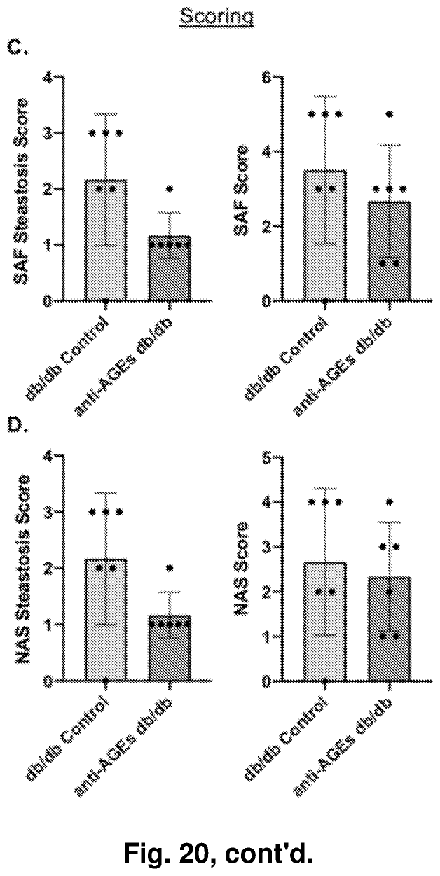

Additionally, db/db mice showed improvement in cardiovascular function when treated with the anti-AGEs compound (FIG. 19). Echocardiography was performed on all mice three days prior the start of the experiment, at 10 weeks into the experiment, and at 20 weeks, at the end of the experimental timeline using ultrasound technology to gather data on the pumping of the heart. Interestingly, it was found that the cardiac output, once being normalized to the weight of the animal, was the only parameter to be significantly increased in treated animals. Cardiac output is normalized to body weight to account for differences in available blood volume to be circulated. Cardiac output is medically defined as the amount of blood the heart pumps through the circulatory system in one minute. This significant (p<0.05) increase in cardiac output, seen in treated db/db mice by week 20, indicates that their blood is circulating more efficiently. A lack of change in stroke volume, fractional shortening, and ejection fraction indicates that the pathophysiology and anatomy of the heart are unchanged between db/db mice untreated and treated with anti-AGEs compounds.

Anti-AGEs Treatment Reduces Severity of Liver Pathologies in db/db Mice

At the time of euthanization, liver samples were collected from all db/db mice for pathology reports generated during a blind study by collaborators at UCSF. Samples were preserved in 4% PFA before slides were generated of 4-micron sections of coronally sliced samples. Sample slides were stained with H&E staining and trichrome staining before being sent for reports to generate images necessary to quantify gross pathology scoring and steatosis and fibrosis scoring.

Samples were scored using a Steatosis-Activity-Fibrosis (SAF) histological scoring system for non-alcoholic fatty liver (NALD) disease as well as a Clinical Research Networking (CRN) scoring system for non-alcoholic steatohepatitis (NAS).

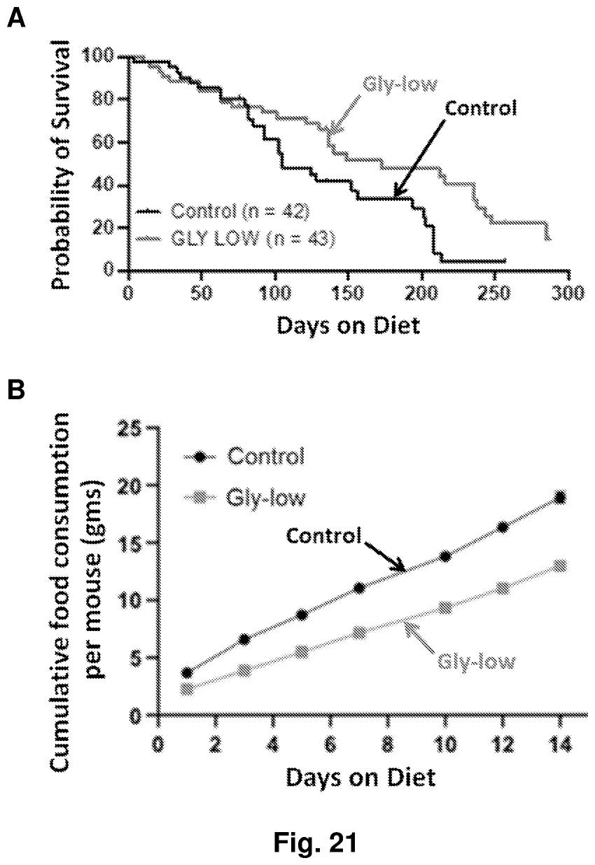

It was observed that livers of untreated db/db mice demonstrated more irregular gross pathology and higher scores of NAS and SAF than db/db mice reared on an ant-AGEs compound cocktail diet (FIG. 20). It is visible by microscopy that untreated db/db mouse livers had increased steatosis, which is a fatty change in the liver, indicated by the globular lipid ‘holes’ in the tissue. Increased SAF scores indicate increased fibrosis of liver tissue, which is an excessive accumulation of proteins, such as collagen, which leads to liver disease.

Only a portion of anti-AGEs treated and untreated db/db mice were randomly chosen at the time of euthanization for liver sample pathology (50% and 30%, respectively, n=6). Each liver was individually characterized for several parameters of either the SAF scoring system, or the NAS scoring system.

The SAF scoring system included parameters such as SAF steatosis grading, lobular inflammation, hepatocellular ballooning, fibrotic activity (SAF activity), and SAF activity. The scoring system and component scores used are published in Hepatology 2012; 56:1751-9. Scores were generated by a pathologist using this metric.

The NASH scoring system included parameters such as NAS steatosis grading, lobular inflammation, hepatocellular ballooning, and NAS activity. The scoring system and component scores used are published in Hepatology 2005; 41:1313-21. Scores were generated by a pathologist using this metric.

### Example 3

### Further Studies of Gly-Low

Late-Life Treatment with Gly-Low Enhances Survival and Improves Fitness in Aged Mice.

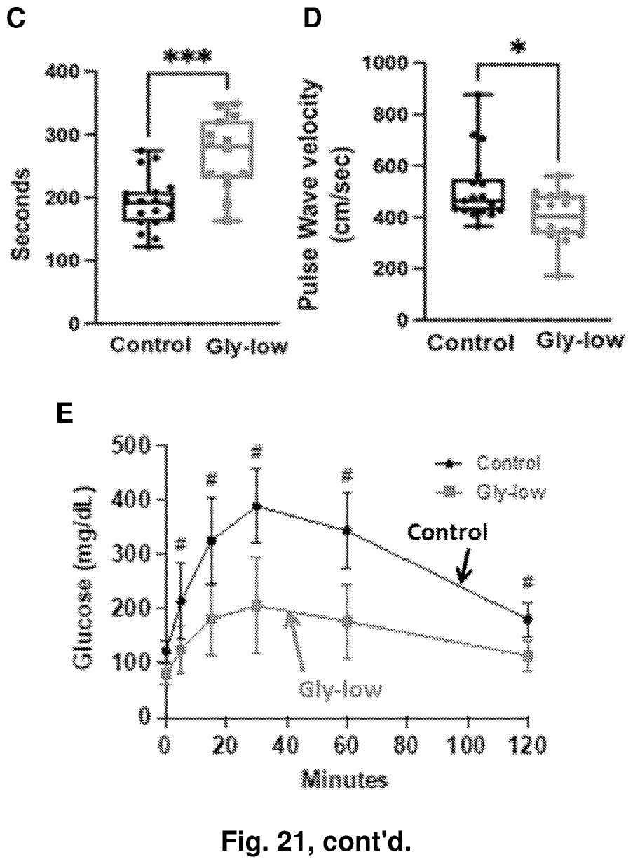

Considering the significant lifespan extension of db/db mice treated with Gly-low, we tested the impact of Gly-low in old C57BL/6 mice starting at 24 months of age. Our preliminary data show a significant increase in lifespan of aged mice treated with Gly-low compared to its control (FIG. 21, panel A). We also observed a significant reduction in food intake and body weight in mice treated for three months (FIG. 21, panel B and data not shown). A recently published study argued that later life induction of dietary restriction (DR) does not benefit from refractive nutritional memory against DR in aged mice (Hahn et al. (2019) Nat. Metab. 1(11): 1059-1073). Thus, our data argue that the lifespan extension effects of Gly-low are not just due to reduced calorie intake but engagement of other biological pathways.

Our preliminary analysis showed Gly-low treatment enhanced the activity of mice (not shown) and improved their resilience in a rotarod run compared to the control diet group (FIG. 21, panel C). Further testing for vasculature health showed decreased pulse wave velocity which increases with age is lowered in Gly-low treated mice compared to controls (FIG. 21, panel D). We also observed Gly-low treatment to improve glucose tolerance in middle-aged mice (10 months) (FIG. 21, panel E), suggesting the ability of Gly-low to engage multiple pathways to improve overall health in aged mice.

### Gly-Low Compounds Reduce Inflammation and Enhance Neurotrophic Signaling.

Our preliminary data show that dietary administration of Gly-low can cause significant changes in gene expression in the hypothalamus (see, e.g., Table 3).

TABLE 3
Signaling pathway analysis of gene expression
changes in the hypothalamus of Gly-low treated mice.
The significantly changed genes in our hypothalamic
dataset as assessed by Ingenuity Pathway Analysis. Each
neurotrophic regulator is associated with positive activation
z-scores (suggesting activation) while inflammatory regulators
are associated with negative activation z-scores (suggesting inhibition).
Pathway Analysis

| | p-value of overlap |
|---|---|
| Neurotrophic/neuroprotective | |
| regulators | |
| beta-estradiol | 1.2 × 10⁻⁹ |
| NRG (Family) | 2.7 × 10⁻⁶ |
| BDNF | 1.0 × 10⁻⁵ |
| NGF | 4.9 × 10⁻² |
| HSF1 | 5.6 × 10⁻⁷ |
| GEPR1 | 3.5 × 10⁻⁴ |
| Inflammatory regulators | |
| NFκB (complex) | 1.3 × 10⁻³ |
| TLR4 | 2.6 × 10⁻³ |
| TNF | 4.6 × 10⁻³ |
| P38 MAPK | 6.2 × 10⁻³ |
| STAT3 | 6.5 × 10⁻³ |

We used a Salmon quasi-mapper (Patro et al. (2017) Nat. Meth. 14(4): 417-419) to quantify bulk RNA sequencing of hypothalami from three samples each from db/db mice and db/db mice treated with Gly-low compounds. We used DEseq2 (Love et al. (2014) Genome Biol. 15(12): 550) to derive significantly differentially expressed genes and associate their fold changes between the two groups. We found 2022 altered genes (FDR<0.5) considerably. We performed gene ontology (GO) analysis using panther (Mi et al. (2013)Nat Protoc. 8(8): 1551-1566) to the significantly changed genes and saw a striking increase in upregulation of genes involved in neuron differentiation, neurogenesis, synaptic transmission, and neuromuscular process.

Gly-low also upregulated genes involved in the secretion of neurotransmitters (acetylcholine and dopamine) and chaperones. We also observed decreased expression of a genes involved in hormonal signaling (GH, FSH, and TSH) and many neuroinflammatory pathways involved in aging and age-related diseases (e.g., NFkB, p38/MAPK, TNF, and Stat3) (see, e.g., Table 3). Furthermore, Gly-low treatment downregulated expression of markers characteristic of activated astrocytes and upregulated in AD and aged brains (C3 p_{adj}<9e⁻⁶; Serpina3n p_{adj}<0.02). The complement component C3 mediates synaptic loss and Aβ toxicity early in AD (Hong et al. (2106) Science, 352(6286): 712-716) and its deficiency protects against an age-related hippocampal decline in wild-type mice and neurodegeneration in an AD model (Hong et al. (2106) Science, 352(6286): 712-716; Wang et al. (2011) Am J Pathol. 178(4): 1509-1516).

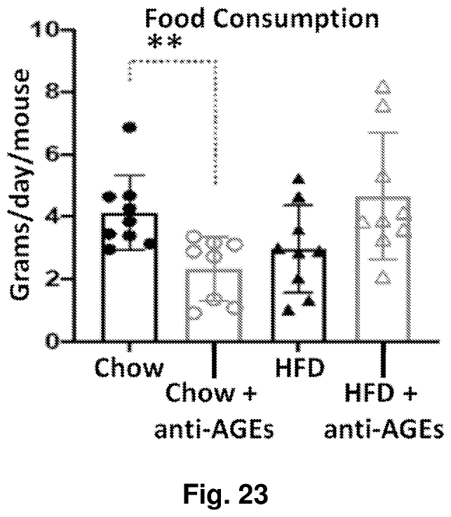

Gly-low treatment also reduced the expression of several matrix metalloproteinases (MMP3). A delicate balance in the expression of MMPs and their inhibitors in brain tissues plays a crucial role in the development of many neurodegenerative diseases, including AD (Brkic et al. (2015) Mediators Inflamm., 2015: 620581). Specifically, Gly-low reduced the expression of MMP3, which is an activator of MMP9. Overexpression of MMP9 is found in AD patients' hippocampus and cerebral cortex and inhibiting MMP-9 improves A$ mediated neurotoxicity and cognitive impairment in mice models. Pathway analysis indicates that Gly-low treatment upregulates several neurotrophic pathways that are compromised in AD brains, such as brain-derived neurotrophic factor (BDNF) (Schindowski et al. (2008) Genes Brain Behav. 7(Suppl 1): 43-56), neuregulin (NRG) (Wang et al. (2014) J. Neural Transm. (Vienna), 121(2): 183-192), and beta-estradiol (Zhao et al. (2015) Ageing Res. Rev. 24(Pt B): 178-190), (see, e.g., Table 3). Each of these genes is implicated in mediating protective responses in models of AD. For instance, estrogen levels are reduced in the brains of female AD patients (Yue et al. (2005) Proc. Natl. Acad. Sci. USA, 102(52): 19198-19203), and several animal studies have shown that estrogens attenuate AD pathology, while estrogen deficiency accelerates it (Yue et al. (2005) Proc. Natl. Acad. Sci. USA, 102(52): 19198-19203; Yun et al. (2018) Brain Behav. Immun. 73: 282-293). Notably, estradiol treatment in mice has been shown to extend lifespan in the mouse ITP trial (Harrison et al. (2021) Aging Cell, 20(5): e13328). To validate whether Gly-low influences AGEs, we examined several genes involved in AGE detoxification and found several primary genes involved in these processes to be upregulated in the hypothalamus (FIG. 23). Thus, up-regulation of these pathways would have tremendous therapeutic potential and supports the rationale to test Gly-low to slow AD related pathology.

### Gly-Low Combination Reduced Food Intake in High Carbohydrate but not High-Fat Diets.

Next, we asked whether the Gly-low combination was capable of reducing food intake in normal chow and high-fat diets in C57BL/6 mice. FIG. 23 shows an acute reduction in food intake among chow-fed mice over 5 days of treatment with the Gly-low combination in metabolic cages. To our surprise, this acute effect did not manifest when tested in C57BL/6 mice fed a high-fat diet, which is well established to induce obesity (Lloret et al. (2019) Front. Neurosci. 13: 508) (FIG. 23). We obtained consistent findings when examining oxygen consumption and energy expenditure. Gly-low reduced these parameters when mice were fed a normal chow diet, but not when fed a high-fat diet (data not shown). In pondering this acute dietary impact on the effects of Gly-low, we noted that the normal chow diet we used (Envigo, AIN-93G) included 60% calories from carbohydrates and only 21% from fat, whereas the high-fat diet (TD.06414) included 60% of calories from fat and only 21% from carbohydrates. Given that MGO is a byproduct of glycolysis and our preliminary data, we hypothesize that lowering MGO and downstream AGEs would preferentially reduce carbohydrate consumption.

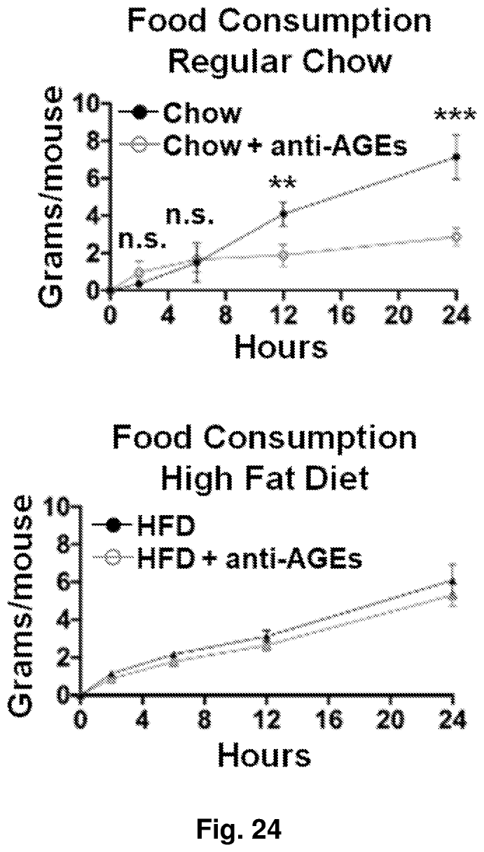

We wanted to confirm that our Gly-low combination did not impact food intake by inducing conditioned taste aversion. We observed that in mice allowed to refeed after a prolonged fast, the presence or absence of Gly-low did not reduce food consumption over the first six hours, although there was an expected reduction in food intake later on (FIG. 24, top panel). Moreover, the Gly-low combination did not alter food intake when a high-fat diet was used for refeeding, which would not have been expected if the effects were due to taste aversion (FIG. 24, bottom panel). Most importantly, injecting our Gly-low combination intraperitoneally (i.p.) was also sufficient in lowering food intake (data not shown), which would also not be expected if this effect was due to taste aversion.

### Gly-Low Treatment Reduces Food Intake and Body Weight and Enhances Working Memory in the 3×T2-AD Model.

Considering the significant upregulation of genes involved in neuroprotection and reduced inflammatory marker gene expression in the hypothalamus of Gly-low treated db/db mice, we hypothesized that Gly-low treatment would rescue disease pathology in the mouse model for neurodegeneration. To this end, we utilized the 3×Tg-AD mice model that is widely accepted as a progressive model for AD (Oddo et al. (2003) Neuron, 39(3): 409-421). The 3×Tg-AD mice contain three mutations associated with familial AD (APP Swedish, MAPT P301L, and PSEN1 M146V). Translation of the overexpressed transgenes appears restricted to the central nervous system, including the hippocampus and cerebral cortex. These mice display both plaque and tangle pathology. Aβ deposition is progressive, with intracellular immunoreactivity detected in some brain regions as early as three to four months of age. Extracellular Aβ deposits appear by six months in the frontal cortex and become more extensive by twelve months. Changes in tau occur later; by 12 to 15 months, aggregates of conformationally-altered and hyperphosphorylated tau are detected in the hippocampus.

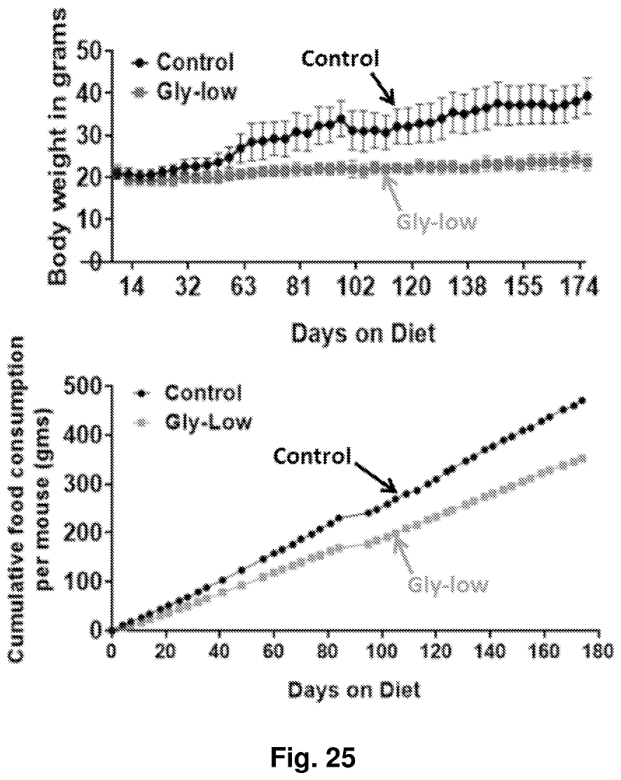

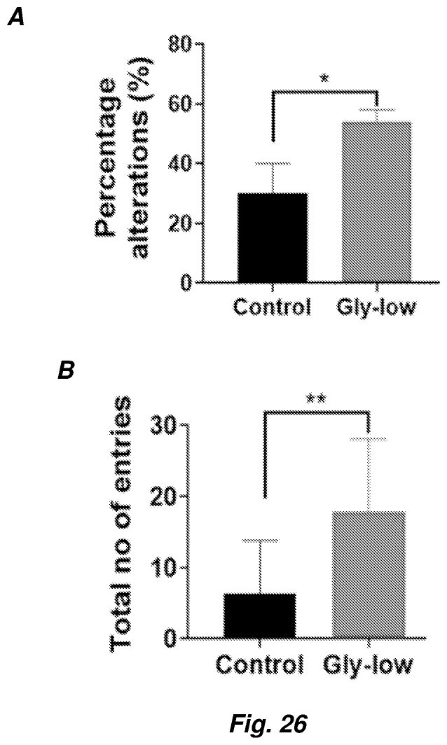

We started Gly-low treatment in both male and female 3×Tg-AD mice at the age of 12 weeks. Food consumption and body weight were measured twice a week, mice were maintained at 12 hr light/dark cycle. Similar to the results that we observed in db/db mice and the aged cohort sample, we found a significant alteration in food intake and body weight of Gly-low treated 3×Tg-AD mice cohort compared to its corresponding control. The female mice showed a robust response to Gly-low treatment compared to the male mice of the same age (FIG. 25). Gly-low treatment also improved spatial learning and memory in the Y-maze test (Kraeuter et al. (2019) Meth. Mol. Biol. 1916: 105-111) (FIG. 26).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A formulation for inducing or increasing weight loss or reducing or preventing weight gain in a mammal, said formulation comprising:
a combination of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, and pyridoxamine and/or a pyridoxamine analog.

2. The formulation of claim 1, wherein said combination of at least two agents is a synergistic combination.

3. The formulation according to any one of claims 1-2, wherein said combination of agents comprises lipoic acid or an analog thereof or a pharmaceutically acceptable salt of said lipoic acid or analog.

4. The formulation of claim 3, wherein said combination of agents comprises lipoic acid.

5. The formulation of claim 3, wherein said combination of agents comprises a lipoic acid analog.

6. The formulation of claim 5, wherein said lipoic acid analog comprises an analog selected from the group consisting of bisnor-lipoic (1,2-dithiolane-3-propanoic), and tetranorlipoic (1,2-dithiolane-3-carboxylic) acid, and a lipoic acid amide (e.g., 6,8-dithiooctanoic amide, 2-(N,N-dimethylamine) ethylamido lipoate, etc.).

7. The formulation according to any one of claims 1-6, wherein said lipoic acid or lipoic acid analog comprises a substantially pure R enantiomer.

8. The formulation according to any one of claims 1-6, wherein said lipoic acid or lipoic acid analog comprises a substantially pure S enantiomer.

9. The formulation according to any one of claims 1-8, wherein said combination of agents comprises nicotinamide or a nicotinamide analog or a pharmaceutically acceptable salt of said nicotinamide or nicotinamide analog.

10. The formulation of claim 9, wherein said nicotinamide or nicotinamide analog or nicotinamide metabolite comprises nicotinamide.

11. The formulation of claim 9, wherein said nicotinamide or nicotinamide analog or nicotinamide metabolite comprises a nicotinamide metabolite.

12. The formulation of claim 11, wherein said nicotinamide metabolite comprise nicotinamide mononucleotide (NMN) or nicotinamide ribonucleoside (NMR).

13. The formulation of claim 9, wherein said nicotinamide or nicotinamide analog comprises nicotinic acid (pyridine-3-carboxylic acid), nicotinamide (nicotinic acid amide), or inositol hexanicotinate).

14. The formulation of claim 9, wherein said nicotinamide or nicotinamide analog comprises a nicotinamide analog.

15. The formulation of claim 14, wherein said nicotinamide or nicotinamide analog comprises a nicotinamide analog nicotinamide analog shown in Table 1.

16. The formulation according to any one of claims 9-15, wherein said nicotinamide or nicotinamide analog comprises a substantially pure R enantiomer.

17. The formulation according to any one of claims 9-15, wherein said nicotinamide or nicotinamide analog comprises a substantially pure S enantiomer.

18. The formulation according to any one of claims 1-17, wherein said combination of agents comprises thiamine or a thiamine analog or a pharmaceutically acceptable salt of said thiamine or thiamine analog.

19. The formulation of claim 18, wherein said thiamine or a thiamine analog comprises thiamine.

20. The formulation of claim 18, wherein said thiamine or a thiamine analog comprises a thiamine analog.

21. The formulation of claim 18, wherein said thiamine analog comprises a thiamine analog selected from the group consisting of pyrithiamine, thiamine disulphide and acetylthiamine, oxythiamine, thiamine diphosphate, oxythiamine diphsophate, neopyrithiamine, 4-methyl-5-(hydroxymethyl)-thiazole, β-(4-Methylthiazolyl-5)-alanine, and 3-(2, 4-dioxo-1, 2, 3, 4-tetrahydro-5-pyrimidinyl) methyl-4-methyl-5-(2-hydroxyethyl) thiazolium nitrate.

22. The formulation according to any one of claims 18-21, wherein said thiamine or thiamine analog comprises a substantially pure S enantiomer.

23. The formulation according to any one of claims 18-21, wherein said thiamine or thiamine analog comprises a substantially pure R enantiomer.

24. The formulation according to any one of claims 1-23, wherein said combination of agents comprises piperine and/or a piperine analog or a pharmaceutically acceptable salt of said piperine and/or piperine analog.

25. The formulation of claim 24, wherein said piperine or a piperine analog comprises piperine.

26. The formulation of claim 24, wherein said piperine or a piperine analog comprises a piperine analog.

27. The formulation of claim 26, wherein said piperine analog comprises a piperine analog selected from the group consisting of 1-(3,4-methylenedioxyphenyl)-penta-2E,4E-dienoic acid methyl ester, 1-E,E-piperinoyl-isobutylamine, and 1-(3,4-methylenedioxyphenyl)-pentanoic acid cyclohexyl amide.

28. The formulation of claim 26, wherein said piperine analog comprises a piperine analog shown in Table 2.

29. The formulation of claim 26, wherein said piperine analog comprises a piperine analog selected from the group consisting of P1057, P622, P545, P725, P557, P2, P4, P1045, P1090, P8, P594, P1087, P1088, P28, P12, P1118, P1, P1122, P677, P1120, P1121, P6, P1112, P1119, P1117, P1116, P1070, P1114, P569, P32, P5, P1084, P1069, P593, P10, P9, P665, P7, P17, P1123, P11, P1080, P33, P743, P16, P1078, P27, P604, P25, P29, 1073, P26, P30, P707, P752, P22, P670, P23, P21, P546, P3, P581, P15, P636, P20, P737, P649, and P689.

30. The formulation according to any one of claims 24-29, wherein said piperine or a piperine analog comprises a substantially pure S enantiomer.

31. The formulation according to any one of claims 24-29, wherein said piperine or a piperine analog comprises a substantially pure R enantiomer.

32. The formulation according to any one of claims 1-31, wherein said combination of agents comprises pyridoxamine and/or a pyridoxamine analog or a pharmaceutically acceptable salt of said pyridoxamine or pyridoxamine analog.

33. The formulation of claim 32, wherein said pyridoxamine or a pyridoxamine analog comprises pyridoxamine.

34. The formulation of claim 32, wherein said pyridoxamine or a pyridoxamine analog comprises a pyridoxamine analog.

35. The formulation of claim 34, wherein said pyridoxamine analog comprises a pyridoxamine analog selected from the group consisting of salicylamine (o-hydroxybenzylamine), thiosalicylamine (o-mercaptobenzylamine), and 3-hydroxy-4-aminomethylpyridine, 3-hydroxy-4-aminomethylpyridine, 1-methylpyridoxamine chloride, 1-methyl-3-hydroxy-4-aminomethylpyridinium chloride, pyridoxamine phosphate, pyridoxal 5 phosphate, pyridoxine, and an alkyl-pyridoxamine (alkyl-PM).

36. The formulation of claim 34, wherein said pyridoxamine analog comprises an alkyl pyridoxamine.

37. The formulation of claim 36, wherein said pyridoxamine analog comprises a pentyl-PM, a hexyl-PM, or a heptyl-PM.

38. The formulation according to any one of claims 32-37, wherein said pyridoxamine or pyridoxamine analog comprises a substantially pure S enantiomer.

39. The formulation according to any one of claims 32-37, wherein said pyridoxamine or pyridoxamine analog comprises a substantially pure R enantiomer.

40. The formulation according to any one of claims 1-39, wherein said agents comprising said combination of two or more agents comprises lipoic acid, thiamine, nicotinamide, piperine, and pyridoxamine in substantially equal amounts.

41. The formulation according to any one of claims 1-39, wherein said agents comprising said combination of two or more agents comprises lipoic acid, thiamine, nicotinamide, and pyridoxamine in substantially equal amounts, and piperidine in a greater amount the other four compounds.

42. The formulation according to any one of claims 1-41, wherein the lipoic acid and/or lipoic acid analog, when present in said formulation, is provided at an amount of at least 50 mg/dose, or at least 100 mg/dose, or at least 150 mg/dose.

43. The formulation of claim 42, wherein the lipoic acid and/or lipoic acid analog, when present is administered in an amount of about 150 mg/dose.

44. The formulation according to any one of claims 1-43, wherein the nicotinamide and/or nicotinamide analog, or nicotinamide metabolite, when present in said formulation, is provided at an amount of at least 100 mg/dose, or at least 150 mg/dose, or at least 200 mg/dose.

45. The formulation of claim 44, wherein the nicotinamide and/or nicotinamide analog, or nicotinamide metabolite, when present, is administered in an amount of about 200 mg/dose.

46. The formulation according to any one of claims 1-45, wherein the piperine or piperine analog, when present in said formulation, is provided at an amount of at least 5 mg/dose, or at least 10 mg/dose, or at least 15 mg/dose.

47. The formulation of claim 46, wherein the piperine or piperine analog, when present, is administered in an amount of about 15 mg/dose.

48. The formulation according to any one of claims 1-47, wherein the pyridoxine or pyridoxine analog, when present in said formulation, is provided at an amount of at least about 10 mg/dose, or at least about 25 mg/dose, or at least about 50 mg/dose.

49. The formulation of claim 48, wherein the pyridoxine or pyridoxine analog, when present is administered in an amount of about 50 mg/dose.

50. The formulation according to any one of claims 1-49, wherein the thiamine and/or a thiamine analog, when present in said formulation, is provided at an amount of at least 50 mg/dose, or at least 100 mg/dose.

51. The formulation of claim 50, wherein the thiamine and/or thiamine analog is administered in an amount of about 100 mg/dose.

52. The formulation of claim 1, wherein said formulation comprises:
alpha lipoic acid;
nicotinamide;
thiamine provided as thiamine mononitrate (vitamin B1); (thiamine) piperine; and
pyridoxamine as pyridoxine HCL.

53. The formulation of claim 52, wherein said formulation does not contain additional vitamins or dietary supplements.

54. The formulation according to any one of claims 52-53, wherein the metabolically active ingredients in said formulation consist of:
alpha lipoic acid;
nicotinamide;
thiamine provided as thiamine mononitrate (vitamin B1);
piperine; and
pyridoxamine as pyridoxine HCL.

55. The formulation according to any one of claims 52-54, wherein said alpha lipoic acid is present in an amount ranging from about 100 mg up to about 200 mg per unit formulation.

56. The formulation according to any one of claims 52-55, wherein said nicotinamide is present in an amount ranging from about 100 mg up to about 300 mg per unit formulation.

57. The formulation according to any one of claims 52-56, wherein said thiamine mononitrate is present in an amount ranging from about 50 mg up to about 200 mg per unit formulation.

58. The formulation according to any one of claims 52-57, wherein said pyridoxamine HCL is present in an amount ranging from about 25 mg up to about 100 mg per unit formulation.

59. The formulation according to any one of claims 52-58, wherein said piperine is present in an amount ranging from about 5 mg up to about 25 mg per unit formulation.

60. The formulation according to any one of claims 52-59, wherein a unit formulation comprises:
about 150 mg alpha lipoic acid;
about 200 mg nicotinamide;
about 100 mg thiamine mononitrate;
about 15 mg piperine; and
about 50 mg pyridoxine HCL.

61. The formulation according to any one of claims 52-60, wherein said formulation further comprises a binder.

62. The formulation of claim 61, wherein said binder comprises microcrystalline cellulose.

63. The formulation according to any one of claims 52-62, wherein said formulation further comprises a metallic salt (boundary lubricant).

64. The formulation of claim 63, wherein said metallic salt comprises magnesium stearate.

65. The formulation according to any one of claims 52-64, wherein said formulation further comprises silicon dioxide.

66. The formulation according to any one of claims 52-65, wherein a unit dosage formulation comprises a gelatin capsule.

67. A method for inducing or increasing weight loss or reducing or preventing weight gain in a mammal, said method comprising:
administering to said mammal an effective amount of a combination of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

68. The method of claim 67, wherein combination of agents in said formulation provides a synergistic effect in the induction or increase of weight loss or the reduction or prevention of weight gain.

69. The method according to any one of claims 67-68, wherein said method comprises a method of inducing or increasing weight loss.

70. The method according to any one of claims 67-68, wherein said method comprises reducing or preventing weight gain.

71. The method according to any one of claims 67-70, wherein said method reduces carbohydrate consumption by said mammal.

72. The method according to any one of claims 67-71, wherein said method does not substantially alter lipid consumption by said mammal.

73. The method according to any one of claims 67-72, wherein administering does not result in nausea.

74. The method according to any one of claims 67-73, wherein comprises a method for reducing blood glucose in said mammal.

75. The method according to any one of claims 67-74, wherein said method comprises a method for reducing A1C in said mammal.

76. The method according to any one of claims 67-75, wherein said method comprises a method for the treatment or prophylaxis of diabetes.

77. The method of claim 76, wherein said method increases the amount of insulin release in a mammal with diabetes or pre-diabetes, or restores the amount of insulin release in a mammal with diabetes or pre-diabetes to substantially normal levels.

78. A method of ameliorating one or more symptoms of an obesity related disease in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

79. The method of claim 78, wherein said obesity-related disease comprise one or more pathologies selected from the group consisting of nonalcoholic fatty liver disease (NAFLD), high blood pressure, high cholesterol, high blood sugar, heart disease, stroke, and obesity-related cancer.

80. The method of claim 79, wherein said obesity-related disease comprises NAFLD.

81. The method of claim 80, wherein said obesity-related disease comprises nonalcoholic steatohepatitis (NASH).

82. The method according to any one of claims 78-81, wherein said combination of agents provides a synergistic effect in ameliorating one or more symptoms or, and/or slowing or stopping the progression of, and/or to curing said obesity-related disease.

83. The method according to any one of claims 67-82, wherein said mammal is a mammal identified as having elevated triglycerides.

84. The method according to any one of claims 67-83, wherein said mammal is a mammal diagnosed as pre-diabetic.

85. The method according to any one of claims 67-84, wherein said mammal is a mammal diagnosed as having diabetes.

86. The method of claim 78, wherein said effective amount is an amount sufficient to ameliorate a complication of diabetes selected from the group consisting of diabetic neuropathy, cardiomyopathy, nephropathy, retinopathy, microvascular damage, and early mortality.

87. The method of claim 86, wherein said combination of agents provides a synergistic effect in ameliorating a complication of diabetes selected from the group consisting of diabetic neuropathy, cardiomyopathy, nephropathy, retinopathy, microvascular damage, and early mortality.

88. The method according to any one of claims 67-87, wherein said method, produces a reduction in one or more advanced glycation end products.

89. The method of claim 88, wherein said method produces a reduction in, or slows the accumulation of, glyoxal/GO.

90. The method according to any one of claims 88-89, wherein said method produces a reduction in, or slows the accumulation of, methylglyoxal/MGO.

91. The method according to any one of claims 88-90, wherein said method produces a reduction in, or slows the accumulation of 3-deoxyglucosone/3DG.

92. A method of providing neuroprotection to a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

93. The method of claim 92, wherein said neuroprotection slows the progression, stops the progression and/or ameliorates neuronal damage associated with a neurodegenerative disease.

94. The method of claim 93, wherein said neurodegenerative disease comprises a disease selected from the group consisting of Mild Cognitive Impairment (MCI), Alzheimer's disease, Parkinson's disease, amyotrophic lateral sclerosis, and Huntington's disease.

95. The method according to any one of claims 92-93, wherein said combination of agents provides a synergistic effect in slowing the progression, stopping the progression and/or ameliorating neuronal damage associated with said neurodegenerative disease.

96. A method of improving memory and/or cognition in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

97. The method of claim 96, wherein said mammal is a mammal with age-related diminution in cognition and/or age-related memory loss.

98. A method of improving muscle strength in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

99. The method of claim 98, wherein said mammal is a mammal with age-related muscle wasting (sarcopenia).

100. The method of claim 98, wherein said mammal is a mammal with disease-associated muscle wasting.

101. The method of claim 100, wherein said disease-associated muscle wasting is muscle wasting associated with a pathology selected from the group consisting of amyotrophic lateral sclerosis (ALS), muscular dystrophy (MD), multiple sclerosis (MS), and spinal muscular atrophy.

102. A method of reducing inflammation in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

103. A method of upregulating neurotrophic factor(s) in a mammal, said method comprising:
administering to said mammal an effective amount of at least two agents selected from the group consisting of lipoic acid and/or a lipoic acid analog, nicotinamide and/or a nicotinamide analog or nicotinamide metabolite, thiamine and/or a thiamine analog, piperine and/or a piperine analog, pyridoxamine and/or a pyridoxamine analog.

104. The method of claim 103, wherein said neurotrophic factors comprise one or more factors selected from the group consisting of beta-estradiol, NRG (Family), BDNF, NGF, HSF1, and GEPR1.

105. The method according to any one of claims 67-109, wherein said combination of agents comprises a combination of agents found in the formulation according to according to any one of claims 1-66.

106. The method according to any one of claims 67-105, wherein said method comprises administering to said mammal a formulation according to according to any one of claims 1-66.

107. A method of inducing or increasing feeding and weight gain in a mammal, said method comprising:
administering to said mammal an effective amount of hydroimidazolone N_{δ}-(5-hydro-5-methyl-4-imidazolon-2-yl) ornithine (MG-H1).

108. The method of claim 107, wherein said mammal is a mammal that has a pathology characterized by abnormal weight loss.

109. The method of claim 108, wherein said pathology is selected from the group consisting of dysphagia, painful mouth sores, newly applied orthodontic appliances, or loss of teeth, pyloric stenosis, hiatus hernia, coeliac disease, chronic pancreatitis, Crohn's disease, gastrointestinal infection, gastrointestinal fistulas, carcinoid disorders, intestinal hypermotility, hepatobiliary disease, food intolerance, medication induced weight loss, hyperthyroidism, Addison's disease, cancer (e.g., lymphoma, leukemia, carcinoma, sarcoma), heart failure, chronic respiratory disease, chronic kidney disease, liver failure, rheumatoid arthritis, systemic lupus erythematosus, acute infection, chronic infections (e.g., tuberculosis, HIV, parasitic infections, etc.), drug abuse, heavy smoking, stress-induced weight loss, depression, anorexia nervosa, food phobias, and Parkinson's disease.

110. The method according to any one of claims 107-109, wherein said hydroimidazolone N_{δ}-(5-hydro-5-methyl-4-imidazolon-2-yl) ornithine (MG-H1) is administered via a route selected from the group consisting of oral delivery, isophoretic delivery, transdermal delivery, parenteral delivery, aerosol administration, administration via inhalation, intravenous administration, and rectal administration.

111. The method of claim 110, wherein said MG-H1 is orally administered to said mammal.

112. The method according to any one of claims 107-111, wherein said mammal is a human.

113. The method according to any one of claims 107-111, wherein said mammal is a non-human mammal.

114. A pharmaceutical formulation comprising:
hydroimidazolone N_{δ}-(5-hydro-5-methyl-4-imidazolon-2-yl) ornithine (MG-H1); and
a pharmaceutically acceptable carrier.

115. The formulation of claim 114, wherein said formulation is formulated for administration by a route selected from the group consisting of oral delivery, isophoretic delivery, transdermal delivery, parenteral delivery, aerosol administration, administration via inhalation, intravenous administration, and rectal administration.

116. The formulation of claim 115, wherein said formulation is compounded for oral administration.

117. The formulation of claim 115, wherein said formulation is sterile.

118. The formulation according to any one of claims 114-117, wherein said formulation is a unit dosage formulation.

119. A kit for inducing or increasing weight loss or reducing or preventing weight gain in a mammal, said kit comprising:
at least two agents selected from the group consisting of lipoic acid and/or a lipoic acid analog, nicotinamide and/or a nicotinamide analog nicotinamide or metabolite, thiamine and/or a thiamine analog, piperine and/or a piperine analog, pyridoxamine and/or a pyridoxamine analog.

120. The kit of claim 119, wherein each of said agents are provided in separate containers.

121. The kit of claim 119, wherein at least two of said agents are in the same container.

122. The kit of claim 119, wherein at least three of said agents are in the same container.

123. The kit of claim 119, wherein at least four of said agents are in the same container.

124. The kit of claim 119, wherein at least five of said agents are in the same container.

125. The kit according to any one of claims 119-124, wherein said combination of agents comprises a combination of agents found in the formulation according to according to any one of claims 1-66.

126. The kit of claim 119, wherein said kit comprises a container containing a formulation according to according to any one of claims 1-66.
